SEQUENCE LISTING (42 sequences)

SEQ ID NO: 1 - DNA, 2008 bp, Zea mays
  source 1..2008: mol_type = genomic DNA
gtcgtgcccctctctagagataaagagcattgcatgtctaaagtataaaaaattaccacatatttttttgtcacacttatttgaagtgtagtttatctatctctatacatatatttaaacttcactctacaaataatatagtctataatactaaaataatattagtgttttagaggatcatataaataaactgctagacatggtctaaaggataattgaatattttgacaatctacagttttatctttttagtgtgcatgtgatctctctgttttttttgcaaatagcttgacctatataatacttcatccattttattagtacatccatttaggatttagggttgatggtttctatagactaatttttagtacatccattttattctttttagtctctaaattttttaaaactaaaactctattttagttttttatttaataatttagatataaaatgaaataaaataaattgactacaaataaaacaaataccctttaagaaataaaaaaactaagcaaacatttttcttgtttcgagtagataatgacaggctgttcaacgccgtcgacgagtctaacggacaccaaccagcgaaccagcagcgtcgcgtcgggccaagcgaagcagacggcacggcatctctgtagctgcctctggacccctctcgagagttccgctccaccgttggacttgctccgctgtcggcatccagaaattgcgtggcggagcggcagacgtgaggcggcacggcaggcggcctcttcctcctctcacggcaccggcagctacgggggattcctttcccaccgctccttcgctttcccttcctcgcccgccgtaataaatagacaccccctccacaccctctttccccaacctcgtgttcgttcggagcgcacacacacgcaaccagatctcccccaaatccagccgtcggcacctccgcttcaaggtacgccgctcatcctccccccccccctctctctaccttctctagatcggcgatccggtccatggttagggcccggtagttctacttctgttcatgtttgtgttagagcaaacatgttcatgttcatgtttgtgatgatgtggtctggttgggcggtcgttctagatcggagtaggatactgtttcaagctacctggtggatttattaattttgtatctgtatgtgtgtgccatacatcttcatagttacgagtttaagatgatggatggaaatatcgatctaggataggtatacatgttgatgcgggttttactgatgcatatacagagatgctttttttctcgcttggttgtgatgatatggtctggttgggcggtcgttctagatcggagtagaatactgtttcaaactacctggtggatttattaaaggataaagggtcgttctagatcggagtagaatactgtttcaaactacctggtggatttattaaaggatctgtatgtatgtgcctacatcttcatagttacgagtttaagatgatggatggaaatatcgatctaggataggtatacatgttgatgcgggttttactgatgcatatacagagatgctttttttcgcttggttgtgatgatgtggtctggttgggcggtcgttctagatcggagtagaatactgtttcaaactacctggtggatttattaattttgtatctttatgtgtgtgccatacatcttcatagttacgagtttaagatgatggatggaaatattgatctaggataggtatacatgttgatgtgggttttactgatgcatatacatgatggcatatgcggcatctattcatatgctctaaccttgagtacctatctattataataaacaagtatgttttataattattttgatcttgatatacttggatgatggcatatgcagcagctatatgtggattttttagccctgccttcatacgctatttatttgcttggtactgtttcttttgtccgatgctcaccctgttgtttggtgatacttctgcaggtc

SEQ ID NO: 2 - DNA, 2276 bp, Zea mays
  source 1..2276: mol_type = genomic DNA
gataaccaacacaacattatggtctaactagcttgctcagtatatggttgtaggttcataagaatgaaattgaaatattcaatgcaacaatagtcaaaccaaggctagaaaggacatattttggataagatgaactatgagtagttgatatgcttggatgagttctaaatgcccttaggcaaataattgtgcaccttagaaggattgaaaagcttagattttaaataaggtgcattggagctaaagttgagctagaattggaatctaagttggatatatcaaaaaaatgaagaactatgacttagattagttggaacgactctagatatgtttgtctaagtcatagaaatgctcatgaacatagctaaaataagacaaatagtaaagcccaaaatggacttgagtgagctgctccgcgacgcaacataccgagtctagtgtgcaccgggccagtccagtgggtcatcagacaggtacgcaaagaggcatttttacgggggttgtgtggttggtgcacaagaccagtccgatggtccatcagaccacttgcgtataacaaatgttttgaagagatgaagaccatggcgcaccggacctgtcaggacccgacgatcagttcctagttccaatggctagatgacgtggtagcacacgtggcgaggtctggtgccactgacaaggcggccttccgttagaacaactactgatttcacagcctgacagaggttcggtggtgcatcggtcctatactgttggtggctcggtgcactcgaggaaagtgcaaatttgggggattgtacccaacgactatttggatggttggggggctatgaatacaactccaaccagtccattcaatactcaacaactgagaaataactcatacaagctagagcaacactccaaaagatcaaaagcctcccaagtgccaaatagagagaatcgagcaaatgatatccattctagagtggtttgtgatagtgccttgtgtgagtgtgatagagaagagtgtgttgatgcgatcttgtgctctcgcgcttggagttgtgaactcctattgattataaaggctaccaagaggctacgagtctatggagtgccttgcggagattccagtctcctggattgagttgagcaaaaagaaatgcctcaaatttgatctttagaatcacttgagagggttggaagagacatgtccctttaggacgctatcacaaaccattttcgtattcgtattagaaacaatacaaaaacaatttgactagttttccgaccatttttgtattttccgtttttgagtcatgatggcaccgttttttaccgtttctatataatatgagatacatttagcgtataatttttgcaagttacaaacttttgttgtgttcaagtgaatgaagaagagaaaaacaaatgttttaattactctgaataatatgaatgacgaaaaaggttatttgtgtattttaatatcttgaaaaatagataatatactattttattatgtatttgtgaaaagtataaaatatttgtcttttcgaaaatagtttatcgttttcgatcatttttgaataaccaataactccgtatttcgatcgttgattttcgtttgcaccttgacaatatgaaaacaaaaccaatttagatgcttttttgatcgtgtgacgagcctcaccccatgatgaaaccaaacgcactttaaaccgtctgaaaattcggaaattgcgcccatttagtttctttcctccaaaatctccgccgggtccgtcctctcccgcttccgcttcccctcagccgtcacctccccgctcccaccgccaccaatcgcactcgcagtggctgttggcgtgcgctcgcgtccaaaccaagcaaaccccacaaatcctaccttccgccggcgtcgccaccggccgtgctccctccctccggccccggttgaccctagcctcgcatcaccctaaccctacccccctcccgccgctgccagagcaggggaaaaagactcaggtacgtcccacgcccccatccccgtctaagcgtgcgtgacctgttcgagtgcttctcacgcgctggccgcggtggttgttcgagatcagcgccgcgggtgctctgatctgtgctgtttgtggaaatggcggcaaggccaggtggctcgtcttaggacctccgttttagggctcgtactcgtaggattctcacaagtcatgcttatgtcactgtgagaactttagaggtttctattggtattggtggatgggtacggaattcaggggggc

SEQ ID NO: 3 - DNA, 1813 bp, Setaria italica
  source 1..1813: mol_type = genomic DNA
cacgggtaatgcacgcagccacccaggcgcgcgcgctagcggagcacggtcaggtgacacgggcgtcgtgacgcttccgagttgaaggggttaacgccagaaacagtgtttggccagggtatgaacataacaaaaaatattcacacgaaagaatggaagtatggagctgctactgtgtaaatgccaagcaggaaactcacgcccgctaacatccaacggccaacagctcgacgtgccggtcagcagagcatcggaacactggtgattggtggagccggcagtatgcgccccagcacggccgaggtggtggtggcccgtggccctgctgtctgcgcggctcgggacaacttgaaactgggccaccgcctcgtcgcaactcgcaacccgttggcggaagaaaggaatggctcgtaggggcccgggtagaatcgaagaatgttgcgctgggcttcgattcacataacatgggcctgaagctctaaaacgacggcccggtcgccgcgcgatggaaagagaccggatcctcctcgtgaattctggaaggccacacgagagcgacccaccaccgacgcggaggagtcgtgcgtggtccaacacggccggcgggctgggctgcgaccttaaccagcaaggcacgccacgacccgccccgccctcgaggcataaataccctcccatcccgttgccgcaagactcagatcagattccgatccccagttcttccccaatcaccttgtggtctctcgtgtcgcggttcccagggacgcctccggctcgtcgctcgacagcgatctccgccccagcaaggtatagattcagttccttgctccgatcccaatctggttgagatgttgctccgatgcgacttgattatgtcatatatctgcggtttgcaccgatctgaagcctagggtttctcgagcgacccagttatttgcaatttgcgatttgctcgtttgttgcgcagcgtagtttatgtttggagtaatcgaggatttgtatgcggcgtcggcgctacctgcttaatcacgccatgtgacgcggttacttgcagaggctgggttctgttatgtcgtgatctaagaatctagattaggctcagtcgttcttgctgtcgactagtttgttttgatatccatgtagtacaagttacttaaaatttaggtccaatatattttgcatgcttttggcctgttattcttgccaacaagttgtcctggtaaaaagtagatgtgaaagtcacgtattgggacaaattgatggtttagtgctatagttctatagttctgtgatacatctatctgattttttttggtctattggtgcctaacttatctgaaaatcatggaacatgaggctagtttgatcatggtttagttcattgtgattaataatgtatgatttagtagctattttggtgatcgtgtcattttatttgtgaatggaatcattgtatgtaaatgaagctagttcaggggttacgatgtagctggctttgtattctaaaggctgctattattcatccatcgatttcacctatatgtaatccagagcttttgatgtgaaatttgtctgatccttcactaggaaggacagaacattgttaatattttggcacatctgtcttattctcatcctttgtttgaacatgttagcctgttcaaacagatactgttgtaatgtcctagttatataggtacatatgtgttctctattgagtttatggacttttgtgtgtgaagttatatttcattttgctcaaaactcatgtttgcaagctttctgacattattctattgttctgaaacaggtg

SEQ ID NO: 4 - DNA, 1802 bp, Saccharum officinarum
  source 1..1802: mol_type = genomic DNA
gaattcattatgtggtctaggtaggttctatatataagaaaacttgaaatgttctaaaaaaaaattcaagcccatgcatgattgaagcaaacggtatagcaacggtgttaacctgatctagtgatctcttgcaatccttaacggccacctaccgcaggtagcaaacggcgtccccctcctcgatatctccgcggcgacctctggctttttccgcggaattgcgcggtggggacggattccacaaccgcgacgcaaccgcctctcgccgctgggccccacaccgctcggtgccgtagcctcacgggactctttctccctcctcccccgttataaattggcttcatcccctccttgcctcatccatccaaatcccagtccccaatcccatcccttcgtcggagaaattcatcgaagcgaagcgaatcctcgcgatcctctcaaggtactgcgagttttcgatccccctctcgacccctcgtatgtttgtgtttgtcgtacgtttgattaggtatgctttccctgtttgtgttcgtcgtagcgtttgattaggtatgctttccctgttcgtgttcatcgtagtgtttgattaggtcgtgtgaggcgatggcctgctcgcgtccttcgatctgtagtcgatttgcgggtcgtggtgtagatctgcgggctgtgatgaagttatttggtgtgatctgctcgcctgattctgcgggttggctcgagtagatatggatggttggaccggttggttcgtttaccgcgctagggttgggctgggatgatgttgcatgcgccgttgcgcgtgatcccgcagcaggacttgcgtttgattgccagatctcgttacgattatgtgatttggtttggacttattagatctgtagcttctgcttatgttgccagatgcgcctactgctccatatgcctgatgataatccataaatggcagtggaaatcaactagttgattgcggagtcatgtatcagctacaggtgtagggactagctacaggtgtagggactgcgtctaattgtttggtccttaactcatgtgcaattatgcaatttagtttagatgtttgttccaatcatctaggctgtaaaagggacactggttagattgctgtttaatctttttagtagattatattatattggtaacttattaaccctattacatgccataacgtggattctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatggcacaatttttagtcacttaacccatgcagattgaactggtccctgcatgttttgctaaattgttctattctgattagaccatatatcaggtatttttttttggtaatggttctcttattttaaatgctatatagttctggtacttgttagaaagatctggttcatagtttagttgcctatccttcgaattaggatgctgagcagctgatcctatagctttgtttcatgtatcaattcttttgtgttcaacagtcagtttttgttagattcattgtaacttatgttcgcttactcttctggtcctcaatgcttgcag

SEQ ID NO: 5 - DNA, 1399 bp, Oryza sativa
  source 1..1399: mol_type = genomic DNA
tcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttagaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgccggtaaccaccccgcccctctcctctttctttctccgtttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccgtgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaag

SEQ ID NO: 6 - DNA, 3688 bp, Saccharum officinarum
  source 1..3688: mol_type = genomic DNA
aagttttgctaaaatgaacaaagaattggggaaactatagccaaagtgggtggggaatggtgccaaacaaaacttcgtaaaccaacccaaaaagatccggaaaacaaatggatacgtgcagggcatgcatgcaatagcccagccataaaaagcggcgagccaatgcccgggtgtcaaacaaaatggcgcctgtgccggctctggctgcttccggctcagctttcggaacgatccgccgcagtttggcctcgcatatgatgacgatgatggtctcctcttctcgatttgtagctccggcatgggagccacctcctgtcggctcacacatagcacgcgccttagcccgtgctcgctctcccctagatgcttcacctgcgccaatcagtgtgagcccatcgtgtcagatggtactcgtacgtatggagtaacgtgataccacaacacgtacactggtcagaattgatagtatatgatcctgtcgacccgatgtgttttagtaccttgcagtggccggagaggagtggccgcgcgcatgcggcgcaggggttctccgcgctcgctgatcgcttcctcactgtgcgctcgtttaggaacaccacctcgtggtcgctcaccatgtgtgactgcatgcaacgctacgaatcaggacccagatggaaacgaagcgcctctcgaccacctctgcctcggtgatggttggtgtgcagtgcgtacgcatgcacgctaccaatatcatacctggatgccggtgcaatcgaacagcttcaggttgtcgacgcggacggcgaagcaggacgcgtacttccatatctttgggttccattacgtaccgtcaatcgaataaataaagagaagagtttgagatcagcttgttgggagcaggtgaccgcccgacatgcatgccgattgtcgacggcacggaaataaacaacacatttgtgagggagccagggaggcagtggcggcacagcgtcgcggcacagtcgatgcagaagtggttcttgtcgttcttgcgctccccccgggtgtgcagcgcacgcctttgaaaaactccgatagcaggccacacagccattgcggggcgccgcgcacggccgccagctgcatccccgtttgttcgcacatgcgctaggtggtcctgcggccgttccttgcaccgcggagacgcggggtggaccagtgggggaatggatgaactgctggtaggtttggttggattggcgagtgcgtagagggggcatgggcaacgatagactcgattcaattcaaagactgaaaatagtggagttctaacaccattctgtgcggcgctaattctcgacatggcaggcgtaagcataataccgacatggcatgcaacgatgttcgtgaacagtggtgacacatggatatggtggccgtccaggggattcgttccattcaattcaaagaccgaaaatcgcggggttccgtagcattttgtgcggtgctaattctcgaacatgcgagacgtaagcctaataccgagatggcatgcaacaatgttcgtgaacaacagtgacacgtggatgcggtggccgtctagggattcgcgttctaagctggtatatgtgcggtgttaattcttgacatgcggggcgtaagtgtaataccaagatgaacggtgacacgtggacgcgggggtcgtcaaacaattcattccgtggtctagggtaggttatatataaaggccagtcttagtgggggattttatggccatgttattaatgcaacccatatttggaaaacagtgcaggaagagtttcatcttcgtaaaactctctctaattccatgaaactcttatcatctctctcttcatcaatacggtgccacatcagcctatttaatgtccatgaaactctgatgaaatccactgagacgggcctcagaaaacttgaaatcttctaaaaaaaattcaagtccatgcatgattgaagcaaacggtatagcaacggtgttaacctgatctagtgatctcttgtaatccttaacggccacctaccacaggtagcaaacggcgtccccctcctcgatatctccgcggcggcctctggctttttccgcggaattgcgcggtggggacggattcctcgagaccgcgacacaaccgcctttcgccgctgggccccacaccgctcggtgccgtagcctcacgggactctttctccctcctcccccgctataaattggcttcatcccctccttgcctcatccatccaaatcccagtccccaatcccagcccatcgtcggagaaattcatagaagcgaagcgaatcctcgcgatcctctcaaggtagtgcgagttttcgattcccctctcgacccctcgtatgctttccctgtttgtgtttcgtcgtagcgtttgattaggtatgctttccctgtttgtgttcgtcgtagcgtttgatttggtatgctttccccgttcgtgttcctcgtagtgtttgattaggtcgtgtgaggcgatggcctgctcgcatccttcgatctgtagtcgatttgcgggtcgtggtgtagatctgcgggctgtgatgaagttatttggtgtgatcgtgctcgcctgattctgcgggttggctcgagtagatatgatggttggaccggttggtttgtttaccgcgctagggttgggctgggatgatgttgcatgcgccgttgcgcgtgatcccgcagcaggacttgcgtttgattgccagatctcgttacgattatgtgatttggtttggactttttagatctgtagcttctgcttatgtgccagatgcgcctactgctcatatgcctgatgataatcataaatggctgtggaactaactagttgattgcggagtcatgtatcagctacaggtgtagggactagctacaggtgtagggacttgcgtctaaattgtttggtcctgtactcatgttgcaattatgcaatttagtttagattgtttgttccactcatctaggctgtaaaagggacactgcttagattgctgtttaatctttttagtagattatatattatattggtaacttattacccttattacatgccatacgtgacttctgctcatgcctgatgataatcatagatcactgtggaattaattagttgattgttgaatcatgtttcatgtacataccacggcacaattgcttagttccttaacaaatgcaaattttactgatccatgtatgatttgcgtggttctctaatgtgaaatactatagctacttgttagtaagaatcaggttcgtatgcttaatgctgtatgtgccttctgctcatgcctgatgataatcatatatcactggaattaattagttgatcgtttaatcatatatcaagtacataccatggcacaatttttagtcacttaacccatgcagattgaactggtccctgcatgttttgctaaattgttctatttctgattagaccatatatcatgtaattttttttttgggtaatggttctcctattttaaatgctatatagttctggtacttgttagaaaaatctgcttccatagtttagttgcttatccctcgaattatgatgctgagcagctgatcctatagctttgtttcaggtatcaattcttgtgttcaacagtcagtttttgttagattcattgtaacttatggtcgcttactcttctggtcctcaatgcttgcag

SEQ ID NO: 7 - DNA, 3681 bp, synthetic construct
  source 1..3681: mol_type = other DNA
tcgaagctcgagaagttcaccaactgctactcgctgagcaagacgctgcggttcaaggcgatccccgtcgggaagacccaggagaacatcgacaacaagcggctcctggtcgaggacgagaagcgcgccgaggactacaagggcgtcaagaagctgctggaccggtactacctctccttcatcaacgacgtcctgcactcgatcaagctcaagaacctgaacaactacatctcgctgttccgcaagaagacacggaccgagaaggagaacaaggagctcgagaacctcgagatcaacctgcgcaaggagatcgcgaaggcgttcaagggcaacgaggggtacaagagcctgttcaagaaagacatcatcgagaccatcctgccggagttcctggacgacaaggacgagatcgcgctggtgaactcgttcaacgggttcaccacggccttcaccgggtttttcgacaaccgggagaacatgttcagcgaggaggccaagtcgaccagcatcgccttccggtgcatcaacgagaacctcacccgctacatcagcaacatggacatcttcgagaaggtggacgccatcttcgacaagcacgaggtccaggagatcaaggaaaagatcctgaactcggactacgacgtggaagacttctttgagggcgagttcttcaacttcgtcctcacccaggagggcatcgacgtctacaacgccatcatcggcggcttcgtgacggagagcggcgagaagatcaagggcctcaacgagtacatcaacctctacaaccagaagactaagcagaagctcccgaagttcaagccgctgtacaagcaagtcctgagcgaccgggagtccctctcgttctacggcgagggctacacgagcgacgaggaggtgctggaggtgttccgcaacacgctgaacaagaacagcgagatcttcagctcgatcaagaaactcgagaagctgttcaagaacttcgacgagtacagcagcgccggcatcttcgtcaagaacgggcccgcgatcagcaccatcagcaaggacatcttcggggagtggaacgtgatccgcgacaagtggaacgccgagtacgacgacatccacctcaagaaaaaggcggtggtcacggagaagtacgaggacgaccgccggaagtccttcaagaaaatcgggagcttcagcctcgagcagctccaggagtacgcggacgccgacctgagcgtggtggagaagctcaaggagatcatcatccagaaggtcgacgagatctacaaggtctacggctcgagcgagaagctgttcgacgcggacttcgtgctggagaagtccctcaagaagaacgacgccgtggtggccatcatgaaggatctgctcgacagcgtgaagtcgttcgagaactacatcaaggcattctttggggagggcaaggagacgaaccgggacgagtccttctacggggacttcgtgctcgcgtacgacatcctcctgaaggtcgaccacatctacgacgcgatccggaactacgtcacgcagaagccctacagcaaggacaagttcaagctctacttccagaacccgcagttcatgggcgggtgggacaaggacaaggagaccgactaccgggccacgatcctgcggtacgggtccaagtactacctcgccatcatggacaagaagtacgccaagtgcctccagaagattgacaaggacgacgtgaacgggaactacgagaagatcaactacaagctcctcccggggcccaacaagatgctgccgaaggtgttcttcagcaagaagtggatggcctactacaacccctcggaggacatccagaagatatacaagaacggcacgttcaaaaagggggacatgttcaacctgaacgactgccacaagctgatcgactttttcaaggacagcatcagccgctacccgaagtggtcgaacgcctacgacttcaacttctcggagacggagaagtacaaggacattgcgggcttctaccgggaggtggaggagcagggctacaaggtctccttcgagagcgcctccaagaaagaggtggacaagctcgtggaggagggcaagctgtacatgttccagatctacaacaaggacttctcggacaagtcgcacggcaccccgaacctccacacgatgtacttcaagctgctgttcgacgagaacaaccacgggcagatccgcctcagcggcggggcggagctgttcatgcgccgcgcgtccctcaagaaggaggagctggtcgtgcaccccgccaactccccgatcgcgaacaagaaccccgacaaccccaagaagacaaccaccctctcgtacgacgtctacaaggacaagcggttctcggaggaccagtacgagctgcacatcccgatcgccatcaacaagtgccccaagaacatcttcaagatcaacaccgaggtgcgggtgctgctcaagcacgacgacaacccctacgtcatcgggatcgaccgcggcgagcggaacctgctctacatcgtggtcgtggacgggaaggggaacatcgtggagcagtacagcctgaacgagatcatcaacaacttcaacggcatccgcatcaagacggactaccacagcctcctggacaagaaggagaaggagcggttcgaggcgcggcagaactggacctccatcgagaacatcaaggagctgaaggccggctacatcagccaggtcgtgcacaagatctgcgagctcgtggagaagtacgacgcggtgatcgcgctggaggacttgaacagcgggttcaagaactcccgggtcaaggtcgagaagcaggtctaccagaagttcgagaagatgctgatcgacaagctcaactacatggtggacaagaagtccaacccctgcgccaccggcggcgccctcaagggctaccagatcaccaacaagttcgagtccttcaagtcgatgtctacgcagaacgggttcattttctacatcccggcgtggctcaccagcaagatcgacccgagcacgggcttcgtcaacctcctgaagaccaagtacaccagcatcgcggacagcaagaagttcatctcctcgttcgaccgcatcatgtacgtccccgaggaagacctgttcgagttcgccctcgactacaagaacttctcccggacggacgccgactacatcaaaaagtggaagctctacagctacggcaaccggatccgcatcttccgcaaccccaagaagaacaatgtgttcgactgggaggaggtgtgcctgacgagcgcctacaaggagctcttcaacaagtacggcatcaactaccagcaaggggacatccgcgcgctgctctgcgagcagtccgacaaggcgttctactcgtcgttcatggccctgatgagcctcatgctccagatgcgcaacagcatcaccggccggacggacgtggacttcctgatcagcccggtcaagaacagcgacggcattttctacgacagccggaactacgaggcccaggagaacgccatcctccccaagaacgccgacgcgaacggcgcctacaacatcgcgcggaaggtgctgtgggccatcggccagtttaaaaaggcggaggacgagaagctggacaaggtcaagatcgccatcagcaacaaggagtggctcgagtacgcgcagacgagcgtgaagcac

SEQ ID NO: 8 - protein, 1227 aa, unidentified
  source 1..1227: note = Lachnospiraceae bacterium
SKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 9 - DNA, 36 bp, synthetic construct
  source 1..36: mol_type = other DNA
atggcgggatctaagaagagaagaattaaacaagat

SEQ ID NO: 10 - DNA, 33 bp, synthetic construct
  source 1..33: mol_type = other DNA
ggatctaagaagagaagaattaaacaagattga

SEQ ID NO: 11 - DNA, 298 bp, Oryza sativa
  source 1..298: mol_type = genomic DNA
taatcgatcctccgatcccttaattaccataccattacaccatgcatcaatatccatatatatataaaccctttcgcacgtacttatactatgttttgtcatacatatatatgtgtcgaacgatcgatctatcactgatatgatatgattgatccatcagcctgatctctgtatcttgttatttgtataccgtcaaataaaagtttcttccacttgtgttaataattagctactctcatctcatgaaccctatatataactagtttaatttgctgtcaattgaacatgatgatcgatg

SEQ ID NO: 12 - DNA, 280 bp, synthetic construct
  source 1..280: mol_type = other DNA
aatctgtgaagagtttattcacgattaatctaggaggaagcgcgctttgttggctgtaggcccaaagcagcaatcaacagagtgtgtgagggccagctgctggaaggttcggttttttcaacaggcccacacgcgcgggctgggctgggctgggctggccacgttccttcggtagcagttcggtggagcaacgaggacggtggaatcgaagtttagtaccacctcggctgaagatacggagaaagaccagtttataacctgcctctggagcctgctgctc

SEQ ID NO: 13 - DNA, 282 bp, synthetic construct
  source 1..282: mol_type = other DNA
ttaccagctctgtgaaacttcacgaaacgcaatcggaaaacgaggtgggctgtgttacacagctgggcctgtgtcaacagcaacgaagcccacccaacagaggattgggcctcgatcaagcggcctcacaacgtgcggacacgcggtgcgaggccactctgtcagcagaagcccggctgggagcaacgagcaacttgtgcgctgcgctgaaggtttagtaccacatcggttgcgtaacgaccagcttaccaccttataagcttaaaccggagcgctgctttc

SEQ ID NO: 14 - DNA, 27 bp, Zea mays
  source 1..27: mol_type = genomic DNA
tttcatagtacacgtctgggtagattc

SEQ ID NO: 15 - DNA, 27 bp, Zea mays
  source 1..27: mol_type = genomic DNA
tttgatgatgaaggagtgggcgttggc

SEQ ID NO: 16 - DNA, 27 bp, Zea mays
  source 1..27: mol_type = genomic DNA
tttggataaccaaaccagggcctatag

SEQ ID NO: 17 - DNA, 27 bp, Zea mays
  source 1..27: mol_type = genomic DNA
tttgaatgaagccatggaaggagagaa

SEQ ID NO: 18 - DNA, 27 bp, Zea mays
  source 1..27: mol_type = genomic DNA
tttccggcagcgcgtcgtagcagttct

SEQ ID NO: 19 - DNA, 27 bp, Zea mays
  source 1..27: mol_type = genomic DNA
tttggtgtctagtgtgtgcgaagatgg

SEQ ID NO: 20 - DNA, 27 bp, Zea mays
  source 1..27: mol_type = genomic DNA
tttctgggtgggattaggtcactggcg

SEQ ID NO: 21 - DNA, 27 bp, Zea mays
  source 1..27: mol_type = genomic DNA
tttggttgagacatggtccatctaggg

SEQ ID NO: 22 - DNA, 23 bp, Zea mays
  source 1..23: mol_type = genomic DNA
atagtacacgtctgggtagattc

SEQ ID NO: 23 - DNA, 23 bp, Zea mays
  source 1..23: mol_type = genomic DNA
atgatgaaggagtgggcgttggc

SEQ ID NO: 24 - DNA, 23 bp, Zea mays
  source 1..23: mol_type = genomic DNA
gataaccaaaccagggcctatag

SEQ ID NO: 25 - DNA, 23 bp, Zea mays
  source 1..23: mol_type = genomic DNA
aatgaagccatggaaggagagaa

SEQ ID NO: 26 - DNA, 23 bp, Zea mays
  source 1..23: mol_type = genomic DNA
cggcagcgcgtcgtagcagttct

SEQ ID NO: 27 - DNA, 23 bp, Zea mays
  source 1..23: mol_type = genomic DNA
gtgtctagtgtgtgcgaagatgg

SEQ ID NO: 28 - DNA, 23 bp, Zea mays
  source 1..23: mol_type = genomic DNA
tgggtgggattaggtcactggcg

SEQ ID NO: 29 - DNA, 23 bp, Zea mays
  source 1..23: mol_type = genomic DNA
gttgagacatggtccatctaggg

SEQ ID NO: 30 - DNA, 20 bp, unidentified
  source 1..20: mol_type = genomic DNA; note = Lachnospiraceae bacterium
aatttctactaagtgtagat

SEQ ID NO: 31 - DNA, 27 bp, Zea mays
  source 1..27: mol_type = genomic DNA
tttggcgttccattcaggttagtggac

SEQ ID NO: 32 - DNA, 2523 bp, Zea mays
  source 1..2523: mol_type = genomic DNA
atgcagcagcagcagcagcgcttagacataggcaagctacaagaggtagtgaagacccgctggctaaagcctcaagaagttctcaagatactgcagaatcatgagctgttcacgatttcgcataagccccctcagaagccacagagtggttcttggtttcttttcaaccgtagagtactccggtacttccgaaatgatggatttgaatggcaaaagaagagaaatgggaagaccattaatgaagcccatgagcgccttaaggttgataatgtggatgcactgaattgctactatgctcgtggagacaaaaatccaacatttcagaggcggatatattggatgcttgatccggcttatgaacacattgtccttgtccactatagggatgttctagagggcagcatttcagtgtctgcacgaaatgattcttcaacattgaatcagaatggcagtgctagcagagctgaagtacatagttcacctgattggacaagcgagttaattgcgcattgtactaattcgtgcagtccgggatctgctgaagaagtcagttcccaaattagtgcaagtgaatctgatttgattcaacataaggcagccttgcggaagcttaaaatgcagctgagtctggaagacaaagaagattgtgatgttaatgctgaggatgttccagcagataatgagcctattattttacctgtgattcagaatgaggagccaggcacttctaggaatcatgatgatatctttgatgtgctggactttagtgaagatcatatcaacggaactggaacccatccatgtcacagtgctattgacgtgctaaaaaattcagacacatggttagaggatgatcaacttgaggcaattctgcatccagcgtgtatgacattgactgaaaatcaatggttcaggatacatgaggtttctccagaatcagcgttttcttatgaaagtacaaaggttatcattgtaggagacttcctttgcaatccccctcacagttcatggcaagtgctgtttggtgatgttaaggtatgtgtggaaattatccagcaaggtgtcatccgttgccatactccatgccttgatgctggaaaagtgagaatgtgcttgcttgatgggaatgggaagttctgcagtgaagcacgagaatttgaattccttgaaaagcctaccaaatgcatgattgatggaaacaggaacctctgcaatgaagcacaagatgtcaaactgcatcagatacctaccaaaagtagtgaggagctgtcactgttactacattatgtgcatacagtttttgatggtcatgctagtgggctattttcaaattttagcttgccactccaaaatcttggctgtggaatccaaagcaatcaaattgatgttatgaagaaggcgtataagcagctggaccctgagaatacgataagtagtgtcatggaagtattgcttaatgacaagtttaagcaatggttgtcatccaaacagaatattgatggagaccatttgcttcctaagcaataccacaatattatacatacggttgcagccttgggatatgatttggctttgaagccactgcttagttcaggagtgcctataaactaccgtgatgcaaatggatggactgctctacattgggctgcaaggtttggcagagaagatatggtggtggctcttcttactgctggtgctgcagctggtgcactttcacatccgacatcagaggatcctgctgccaaaacacctgcttcaatagctttagcatatggtttcaagggcctatccgcattcctttcagaagcacaattaacaactcatcttgattctatcgagtcaaaagaaaatggaaaacttgattctagggaagaggggatatgtcgtgctgtggacagaatattagacaaaagcagccacgtgcatggtggaactgatgatcagcttgcgcttaaggattccctgggagctgtccggaatgcagttcaggctgcagggcgcatacaagctgcattccgaattttttcctttaagaagaagaaagagatggctcttgggaacagaaatagctgctgcttgtccattagtgaggcaggagcagtttcacatgatatgctggagaaagctgtattatctatccagaagaacttcaggtgctggaaaaaacgtaaagaattcctgaagatgcgaaataatgttgtcaggatacaggcccgggtgcgagctcaccaagaaagaaacaagtacaaggaattgatttcaagtgtcggaatccttgagaaggtgatgatacggtggtaccataaaggtgttggtctgcgtggattcaattctggggcaatgacaatcgatgaagaggtggacgaagacgttgccaaggtcttccggaagctcagagtggaaacagccatcgatgaagctgtttcaagggtatcatgtatcattggtagccctaaagcaatgcatcagtaccgaaggatgctcaataggtaccaacaaacaaaggatgatcaggagaagtaa

SEQ ID NO: 33 - DNA, 280 bp, synthetic construct; identical to SEQ ID NO: 12
  source 1..280: mol_type = other DNA

SEQ ID NO: 34 - DNA, 23 bp, Zea mays
  source 1..23: mol_type = genomic DNA
gcgttccattcaggttagtggac

SEQ ID NO: 35 - DNA, 23 bp, Zea mays
  source 1..23: mol_type = genomic DNA
gccagcgggtcttcactacctct

SEQ ID NO: 36 - DNA, 23 bp, Zea mays
  source 1..23: mol_type = genomic DNA
ggaagtaccggagtactctacgg

SEQ ID NO: 37 - DNA, 23 bp, Zea mays
  source 1..23: mol_type = genomic DNA
gtgcagacactgaaatgctgccc

SEQ ID NO: 38 - DNA, 23 bp, Zea mays
  source 1..23: mol_type = genomic DNA
ggaactgacttcttcagcagatc

SEQ ID NO: 39 - DNA, 23 bp, Zea mays
  source 1..23: mol_type = genomic DNA
gtaggtatctgatgcagtttgac

SEQ ID NO: 40 - DNA, 23 bp, Zea mays
  source 1..23: mol_type = genomic DNA
gcatatggtttcaagggcctatc

SEQ ID NO: 41 - DNA, 23 bp, Zea mays
  source 1..23: mol_type = genomic DNA
gcagcaggatcctctgatgtcgg

SEQ ID NO: 42 - DNA, 23 bp, Zea mays
  source 1..23: mol_type = genomic DNA
gggctaccaatgatacatgatac

(19) United States
(12) Patent Application Publication
(10) Pub. No.: US 2026/0098272 A1
(43) Pub. Date: Apr. 9, 2026

(54) METHODS AND MEANS FOR TRANSGENERATIONAL GENOME EDITING IN PLANTS

(71) Applicant: Monsanto Technology LLC, St. Louis, MO (US)

(72) Inventors: Giane Balser, Twin Falls, ID (US); Lisa Kanizay, St. Louis, MO (US); Ramanjot Kaur, O'Fallon, MO (US); Thomas Scott Ream, Wildwood, MO (US); Zarir Vaghchhipawala, Ballwin, MO (US); Chenxi Wu, Ballwin, MO (US)

(21) Appl. No.: 19/350,752

(22) Filed: Oct. 6, 2025

Related U.S. Application Data
(60) Provisional application No. 63/704,419, filed on Oct. 7, 2024.

(51) Int. Cl. C12N 15/82; A01H 1/02; C12N 9/22; C12N 15/11

(52) U.S. Cl. CPC C12N 15/8201; A01H 1/021; C12N 9/226; C12N 15/111; C12N 2310/20

(57) ABSTRACT

Provided are methods and means for transgenerational genome editing in plants, using plants with genome editing nucleic acid constructs encoding RNA-guided nucleases expressed under the control of selected constitutive promoters, together with one or more nucleic acids encoding one or more guide RNAs.

### CROSS-REFERENCE TO RELATED APPLICATIONS

This application claims the priority of U.S. Provisional Application Ser. No. 63/704,419, filed Oct. 7, 2024, the entire disclosure of which is incorporated herein by reference.

### FIELD

The present disclosure relates to compositions and methods for transgenerational genome editing in plants, by providing plants with genome editing nucleic acid constructs encoding RNA-guided nucleases expressed under the control of selected constitutive promoters, together with one or more nucleic acids encoding one or more guide RNAs.

### INCORPORATION OF SEQUENCE LISTING

A sequence listing contained in the file named “MONS601US-revised_ST26” which is 59,581 bytes (measured in MS-Windows®) and was created on Dec. 1, 2025, containing 42 sequences, is filed electronically herewith and incorporated by reference in its entirety.

### BACKGROUND OF THE INVENTION

Genome editing, using RNA guided nucleases, such as CRISPR/Cas proteins, holds great promises for agricultural applications. One cross editing (1XE) in plants combines gene editing and doubled haploid technologies, to enable the efficient generation and integration of novel edits into diverse elite germplasms through crossing. 1XE functions by crossing a transgenic haploid inducer line (comprising genome editing machinery) with an elite line to generate transgene-free edited haploid progeny, followed by doubling the chromosomes of the resulting transgene-free edited haploids, to fix a homozygous edit in the elite genomic background within 2 to 3 instead of 8 to 9 generations. The deployment of 1XE requires high transgenerational editing ability of the CRISPR system, which is defined as the ability of CRIPR/CAS system to remain active and continually produce new edits after multiple generations of selfing or outcrossing to other plants. The transgenerational editing capability of CRISPR can be measured vertically through new edits being produced in progeny plants through selfing lines comprising CRISPR/CAS for multiple generations, or horizontally through new edits being produced in progeny plants through crossing CRISPR/CAS comprising lines with another inbred/elite line without editing machineries.

Historically, highly active constitutive promoters such as Zm.Ubq1, have often been used to express CRISPR effector proteins in plants. However, these promoters empirically showed a loss of ability to continually create new edits in progeny generations beyond the R0 generation, which might be due to the risk of silencing CRISPR effector constructs driven by strong constitutive promoters.

The present invention addresses these shortcomings in the art by providing new compositions and methods for expressing CRISPR/Cas proteins in plants and plant cells, using selected constitutive plant promoters, as described hereinafter.

### SUMMARY OF THE INVENTION

The invention can be summarized as described in any one of the following numbered embodiments.

Embodiment 1. In one aspect, the disclosure provides a method of editing a genome of a plant, or improving editing the genome of a plant, such as editing the genome of a plant other than sequence dependent integration of a transgenic DNA, comprising:
(a) introducing into a plant cell:
   (i) a first nucleic acid sequence encoding a CRISPR effector protein operably linked to a heterologous first constitutive promoter selected from the group consisting of a plant constitutive promoter from the Zea mays Tubulin1 gene (Zm.Tubg1), a plant constitutive promoter from the Setaria italica Ubiquitin 1 gene (SETit.Ubq1), a plant constitutive promoter from the Saccharum officinarum Ubiquitin 4 gene (So.Ubg4), a plant constitutive promoter from the Oryza sativa Actin1 gene (Os.Act) and a plant constitutive promoter from the Saccharum officinarum Ubiquitin 9 gene (So.Ubg9); and
   (ii) a second nucleic acid sequence encoding at least one guide nucleic acid operably linked to a heterologous second promoter, wherein the at least one guide nucleic acid is capable of hybridizing to a target sequence within the plant genome; and
(b) regenerating at least one plant from the plant cell of step (a),
   wherein the CRISPR effector protein and at least one guide nucleic acid form a ribonucleoprotein within at least one cell of the plant, and wherein the ribonucleoprotein generates at least one modification within the target sequence in at least one cell of the plant.

Embodiment 2. In another embodiment, the disclosure provides a method as described in embodiment 1, wherein the heterologous first constitutive promoter is selected from the group consisting of a plant constitutive promoter from the Setaria italica Ubiquitin 1 gene (SETit.Ubq1), a plant constitutive promoter from the Saccharum officinarum Ubiquitin 4 gene (So.Ubg4), a plant constitutive promoter from the Oryza sativa Actin1 gene (Os.Act) and a plant constitutive promoter from the Saccharum officinarum Ubiquitin 9 gene (So.Ubg9).

Embodiment 3. In another embodiment, the disclosure provides a method as described in embodiment 1, wherein the heterologous first constitutive promoter is selected from the group consisting of a plant constitutive promoter from the Setaria italica Ubiquitin 1 gene (SETit.Ubq1), a plant constitutive promoter from the Saccharum officinarum Ubiquitin 4 gene (So.Ubg4), and a plant constitutive promoter from the Saccharum officinarum Ubiquitin 9 gene (So.Ubg9).

Embodiment 4. In this embodiment, a method is provided as described in embodiment 1, wherein the heterologous first constitutive promoter is a plant constitutive promoter from the Setaria italica Ubiquitin 1 gene (SETit.Ubq1).

Embodiment 5. In this embodiment, a method is provided as described in embodiment 1, wherein the heterologous first constitutive promoter is a plant constitutive promoter from the Saccharum officinarum Ubiquitin 4 gene (So.Ubq4).

Embodiment 6. In this embodiment, a method is provided as described in embodiment 1, wherein the heterologous first constitutive promoter is a plant constitutive promoter from the Saccharum officinarum Ubiquitin 9 gene (So.Ubq9).

Embodiment 7. In this embodiment, a method is provided as described in embodiment 1, wherein the heterologous first constitutive promoter comprises a nucleic acid sequence that is at least 85% identical to a nucleic acid sequence selected from the group consisting of SEQ ID NOs:2-6 or a functional fragment thereof.

Embodiment 8. In this embodiment, a method is provided as described in embodiment 1, wherein the heterologous first constitutive promoter comprises a nucleic acid sequence that is at least 90% identical to a nucleic acid sequence selected from the group consisting of SEQ ID NOs:2-6 or a functional fragment thereof.

Embodiment 9. In this embodiment, a method is provided as described in embodiment, wherein the heterologous first constitutive promoter comprises a nucleic acid sequence that is at least 95% identical to a nucleic acid sequence selected from the group consisting of SEQ ID NOs:2-6 or a functional fragment thereof.

Embodiment 10. In this embodiment, a method is provided as described in embodiment 1, wherein the heterologous first constitutive promoter comprises a nucleic acid sequence that is at least 98% identical to a nucleic acid sequence selected from the group consisting of SEQ ID NOs:2-6 or a functional fragment thereof.

Embodiment 11. In this embodiment, a method is provided as described in embodiment 1, wherein the heterologous first constitutive promoter comprises a nucleic acid sequence that is at least 99% identical to a nucleic acid sequence selected from the group consisting of SEQ ID NOs:2-6 or a functional fragment thereof.

Embodiment 12. In this embodiment, a method is provided as described in embodiment 1, wherein the heterologous first constitutive promoter comprises a nucleic acid sequence that is 100% identical to a nucleic acid sequence selected from the group consisting of SEQ ID NOs:2-6 or a functional fragment thereof.

Embodiment 13. In this embodiment, a method is provided as described in any one of embodiments 7 to 12, wherein the plant constitutive promoter from the Zea mays Tubulin1 gene (Zm.Tubg1) gene comprises a nucleic acid sequence that is at least 95% identical to a nucleic acid sequence of SEQ NO: 2 or a functional fragment thereof.

Embodiment 14. In this embodiment, a method is provided as described in any one of embodiments 7 to 12, wherein the plant constitutive promoter from the Setaria italica Ubiquitin 1 gene comprises a nucleic acid sequence that is at least 95% identical to a nucleic acid sequence of SEQ NO: 3 or a functional fragment thereof.

Embodiment 15. In this embodiment, a method is provided as described in any one of embodiments 7 to 12, wherein the plant constitutive promoter from the Saccharum officinarum Ubiquitin 4 gene (So.Ubg4) comprises a nucleic acid sequence that is at least 95% identical to a nucleic acid sequence of SEQ NO: 4 or a functional fragment thereof.

Embodiment 16. In this embodiment, a method is provided as described in any one of embodiments 7 to 12, wherein the plant constitutive promoter from Oryza sativa Actin1 gene (Os.Act) the comprises a nucleic acid sequence that is at least 95% identical to a nucleic acid sequence of SEQ NO: 5 or a functional fragment thereof.

Embodiment 17. In this embodiment, a method is provided as described in any one of embodiments 7 to 12, wherein the plant constitutive promoter from the Saccharum officinarum Ubiquitin 9 gene (So.Ubg9) comprises a nucleic acid sequence that is at least 95% identical to a nucleic acid sequence of SEQ NO: 6 or a functional fragment thereof.

Embodiment 18. In this embodiment, a method is provided as described in any one of embodiments 7 to 12, wherein the plant constitutive promoter from the Zea mays Tubulin1 gene (Zm.Tubg1) gene comprises a nucleic acid sequence that is 100% identical to a nucleic acid sequence of SEQ NO: 2 or a functional fragment thereof.

Embodiment 19. In this embodiment, a method is provided as described in any one of embodiments 7 to 12, wherein the plant constitutive promoter from the Setaria italica Ubiquitin 1 gene comprises a nucleic acid sequence that is 100% identical to a nucleic acid sequence of SEQ NO: 3 or a functional fragment thereof.

Embodiment 20. In this embodiment, a method is provided as described in any one of embodiments 7 to 12, wherein the plant constitutive promoter from the Saccharum officinarum Ubiquitin 4 gene (So.Ubg4) comprises a nucleic acid sequence that is 100% identical to a nucleic acid sequence of SEQ NO: 4 or a functional fragment thereof.

Embodiment 21. In this embodiment, a method is provided as described in any one of embodiments 7 to 12, wherein the plant constitutive promoter from Oryza sativa Actin1 gene (Os.Act) the comprises a nucleic acid sequence that is 100% identical to a nucleic acid sequence of SEQ NO: 5 or a functional fragment thereof.

Embodiment 22. In this embodiment, a method is provided as described in any one of embodiments 7 to 12, wherein the plant constitutive promoter from the Saccharum officinarum Ubiquitin 9 gene (So.Ubg9) comprises a nucleic acid sequence that is at 100% identical to a nucleic acid sequence of SEQ NO: 6 or a functional fragment thereof.

Embodiment 23. In this embodiment, a method is provided as described in any one of embodiments 1 to 22, wherein the CRISPR effector protein is selected from the group of Cas9, Cas12a, Cas12b and Cas X.

Embodiment 24. In this embodiment, a method is provided as described in any one of embodiments 1 to 23, wherein the CRISPR effector protein is a Cas12a effector protein or a Cas12a derived effector protein selected from FnCas12a, LbCas12a, ErCas12a (MAD7®) or AsCas12a or variants thereof.

Embodiment 25. In this embodiment, a method is provided as described in any one of embodiments 1 to 24, wherein the nucleic acid sequence encoding a CRISPR effector protein is codon-optimized for expression in the plant cell.

Embodiment 26. In this embodiment, a method is provided as described in any one of embodiments 1 to 25, wherein the CRISPR effector protein comprises an amino acid sequence having at least 90% sequence identity or at least 95% sequence identity or is identical to the amino acid sequence of SEQ ID NO: 8.

Embodiment 27. In this embodiment, a method is provided as described in any one of embodiments 1 to 26, wherein the nucleotide sequence encoding the CRISPR effector protein comprises a nucleotide sequence having at least 90% sequence identity or at least 95% sequence identity or is identical to the nucleotide sequence of SEQ ID NO: 7.

Embodiment 28. In this embodiment, a method is provided as described in any one of embodiments 1 to 27, wherein heterologous second promoter is a Polymerase III DNA dependent RNA polymerase dependent promotor (POLIII promoter).

Embodiment 29. In this embodiment, a method is provided as described in embodiment 28, wherein the POL III promoter is selected from the group consisting of a U6 promoter, an H1 promoter, a 5S promoter, an Adenovirus 2 (Ad2) VAI promoter, a tRNA promoter, a 7SK promoter, a chimeric POLIII promoter or a synthetic POLIII promoter.

Embodiment 30. In this embodiment, a method is provided as described in any one of embodiments 28 or 29, wherein the POLIII promoter comprises a nucleotide sequence selected from the nucleotide sequence of SEQ ID NO: 12 or SEQ ID NO: 13.

Embodiment 31. In this embodiment, a method is provided as described in any one of embodiments 1 to 30, wherein the plant cell is from a plant selected from a monocotyledonous species, a dicotyledonous species, an angiosperm species or a gymnosperm species.

Embodiment 32. In this embodiment, a method is provided as described in any one of embodiments 1 to 31, wherein the plant cell is from a plant selected from a corn plant, a rice plant, a sorghum plant, a wheat plant, an alfalfa plant, a barley plant, a millet plant, a rye plant, a sugarcane plant, a cotton plant, a soybean plant, a canola plant, a tomato plant, an onion plant, a cucumber plant, an Arabidopsis plant, or a potato plant.

Embodiment 33. In this embodiment, a method is provided as described in any one of embodiments 1 to 32, wherein the plant cell is a corn plant cell.

Embodiment 34. In this embodiment, a method is provided as described in any one of embodiments 1 to 33, wherein the guide RNA is a single guide RNA.

Embodiment 35. In this embodiment, a method is provided as described in any one of embodiments 1 to 34, wherein the guide RNA comprises at least two direct repeat sequences, flanking a spacer comprising a nucleotide sequence complementary to the target sequence.

Embodiment 36. In this embodiment, a method is provided as described in any one of embodiments 1 to 35, wherein said plant regenerated from the plant cell is a haploid inducer.

Embodiment 37. In this embodiment, a method is provided as described in any one of embodiments 1 to 36, further comprising a step of crossing the plant regenerated from the plant cell, to generate a progeny plant.

Embodiment 38. In this embodiment, a method is provided as described in embodiment 37, wherein a least one further modification is generated within a target sequence in the genome of the progeny plant.

Embodiment 39. In this embodiment, a method is provided as described in embodiment 37, wherein said crossing is by self-pollination.

Embodiment 40. In this embodiment, a method is provided as described in embodiment 37, wherein said crossing is by cross-pollination.

Embodiment 41. In this embodiment, a method is provided as described in any one of embodiments 1 to 40, wherein said modification within the target sequence in the plant cell comprises:
(a) insertion of at least one nucleotide;
(b) deletion of at least one nucleotide;
(c) substitution of at least one nucleotide.

Embodiment 42. In another aspect, the disclosure provides a method of editing a genome of a plant comprising:
(a) crossing a first plant with a second plant, wherein the first plant comprises a first nucleic acid sequence encoding a CRISPR effector protein operably linked to a heterologous first constitutive promoter selected from the group consisting of a plant constitutive promoter from the Zea mays Tubulin1 gene (Zm.Tubg1), a plant constitutive promoter from the Setaria italica Ubiquitin 1 gene (SETit.Ubq1), a plant constitutive promoter from the Saccharum officinarum Ubiquitin 4 gene (So.Ubg4), a plant constitutive promoter from the Oryza sativa Actin1 gene (Os.Act) and a plant constitutive promoter from the Saccharum officinarum Ubiquitin 9 gene (So.Ubg9) and wherein the second plant comprises a second nucleic acid sequence encoding at least one guide nucleic acid operably linked to a heterologous second promoter, wherein the at least one guide nucleic acid is capable of hybridizing to a target sequence within the genome; and
(b) obtaining at least one embryo from the crossing of step (a), wherein the CRISPR effector protein and the at least one guide nucleic acid form a ribonucleoprotein within at least one cell of the embryo, and wherein the ribonucleoprotein generates at least one modification within the target sequence in the at least one cell of the embryo.

Embodiment 43. In this embodiment, a method is provided as described in embodiment 42, wherein the heterologous first constitutive promoter is selected from the group consisting of a plant constitutive promoter from the Setaria italica Ubiquitin 1 gene (SETit.Ubq1), a plant constitutive promoter from the Saccharum officinarum Ubiquitin 4 gene (So.Ubg4), a plant constitutive promoter from the Oryza sativa Actin1 gene (Os.Act) and a plant constitutive promoter from the Saccharum officinarum Ubiquitin 9 gene (So.Ubg9).

Embodiment 44. In this embodiment, a method is provided as described in embodiment 42, wherein the heterologous first constitutive promoter is selected from the group consisting of a plant constitutive promoter from the Setaria italica Ubiquitin 1 gene (SETit.Ubq1), a plant constitutive promoter from the Saccharum officinarum Ubiquitin 4 gene (So.Ubg4), and a plant constitutive promoter from the Saccharum officinarum Ubiquitin 9 gene (So.Ubg9).

Embodiment 45. In this embodiment, a method is provided as described in embodiment 42, wherein the heterologous first constitutive promoter is a plant constitutive promoter from the Setaria italica Ubiquitin 1 gene (SETit.Ubq1).

Embodiment 46. In this embodiment, a method is provided as described in embodiment 42, wherein the heterologous first constitutive promoter is a plant constitutive promoter from the Saccharum officinarum Ubiquitin 4 gene (So.Ubq4).

Embodiment 47. In this embodiment, a method is provided as described in embodiment 42, wherein the heterologous first constitutive promoter is a plant constitutive promoter from the Saccharum officinarum Ubiquitin 9 gene (So.Ubq9).

Embodiment 48. In this embodiment, a method is provided as described in embodiment 42, wherein the heterologous first constitutive promoter comprises a nucleic acid sequence that is at least 85% identical to a nucleic acid sequence selected from the group consisting of SEQ ID NOs:2-6 or a functional fragment thereof.

Embodiment 49. In this embodiment, a method is provided as described in embodiment 42, wherein the heterologous first constitutive promoter comprises a nucleic acid sequence that is at least 90% identical to a nucleic acid sequence selected from the group consisting of SEQ ID NOs:2-6 or a functional fragment thereof.

Embodiment 50. In this embodiment, a method is provided as described in embodiment 42, wherein the heterologous first constitutive promoter comprises a nucleic acid sequence that is at least 95% identical to a nucleic acid sequence selected from the group consisting of SEQ ID NOs:2-6 or a functional fragment thereof.

Embodiment 51. In this embodiment, a method is provided as described in embodiment 42, wherein the heterologous first constitutive promoter comprises a nucleic acid sequence that is at least 98% identical to a nucleic acid sequence selected from the group consisting of SEQ ID NOs:2-6 or a functional fragment thereof.

Embodiment 52. In this embodiment, a method is provided as described in embodiment 42, wherein the heterologous first constitutive promoter comprises a nucleic acid sequence that is at least 99% identical to a nucleic acid sequence selected from the group consisting of SEQ ID NOs:2-6 or a functional fragment thereof.

Embodiment 53. In this embodiment, a method is provided as described in embodiment 42, wherein the heterologous first constitutive promoter comprises a nucleic acid sequence that is 100% identical to a nucleic acid sequence selected from the group consisting of SEQ ID NOs:2-6 or a functional fragment thereof.

Embodiment 54. In this embodiment, a method is provided as described in any one of embodiments 48 to 53, wherein the plant constitutive promoter from the Zea mays Tubulin1 gene (Zm.Tubg1) gene comprises a nucleic acid sequence that is at least 95% identical to a nucleic acid sequence of SEQ NO: 2 or a functional fragment thereof.

Embodiment 55. In this embodiment, a method is provided as described in any one of embodiments 48 to 53, wherein the plant constitutive promoter from the Setaria italica Ubiquitin 1 gene comprises a nucleic acid sequence that is at least 95% identical to a nucleic acid sequence of SEQ NO: 3 or a functional fragment thereof.

Embodiment 56. In this embodiment, a method is provided as described in any one of embodiments 48 to 53, wherein the plant constitutive promoter from the Saccharum officinarum Ubiquitin 4 gene (So.Ubg4) comprises a nucleic acid sequence that is at least 95% identical to a nucleic acid sequence of SEQ NO: 4 or a functional fragment thereof.

Embodiment 57. In this embodiment, a method is provided as described in any one of embodiments 48 to 53, wherein the plant constitutive promoter from Oryza sativa Actin1 gene (Os.Act) the comprises a nucleic acid sequence that is at least 95% identical to a nucleic acid sequence of SEQ NO: 5 or a functional fragment thereof.

Embodiment 58. In this embodiment, a method is provided as described in any one of embodiments 48 to 53, wherein the plant constitutive promoter from the Saccharum officinarum Ubiquitin 9 gene (So.Ubg9) comprises a nucleic acid sequence that is at least 95% identical to a nucleic acid sequence of SEQ NO: 6 or a functional fragment thereof.

Embodiment 59. In this embodiment, a method is provided as described in any one of embodiments 48 to 53, wherein the plant constitutive promoter from the Zea mays Tubulin1 gene (Zm.Tubg1) gene comprises a nucleic acid sequence that is 100% identical to a nucleic acid sequence of SEQ NO: 2 or a functional fragment thereof.

Embodiment 60. In this embodiment, a method is provided as described in any one of embodiments 48 to 53, wherein the plant constitutive promoter from the Setaria italica Ubiquitin 1 gene comprises a nucleic acid sequence that is 100% identical to a nucleic acid sequence of SEQ NO: 3 or a functional fragment thereof.

Embodiment 61. In this embodiment, a method is provided as described in any one of embodiments 48 to 53, wherein the plant constitutive promoter from the Saccharum officinarum Ubiquitin 4 gene (So.Ubg4) comprises a nucleic acid sequence that is 100% identical to a nucleic acid sequence of SEQ NO: 4 or a functional fragment thereof.

Embodiment 62. In this embodiment, a method is provided as described in any one of embodiments 48 to 53, wherein the plant constitutive promoter from Oryza sativa Actin1 gene (Os.Act) the comprises a nucleic acid sequence that is 100% identical to a nucleic acid sequence of SEQ NO: 5 or a functional fragment thereof.

Embodiment 63. In this embodiment, a method is provided as described in any one of embodiments 48 to 53, wherein the plant constitutive promoter from the Saccharum officinarum Ubiquitin 9 gene (So.Ubg9) comprises a nucleic acid sequence that is at 100% identical to a nucleic acid sequence of SEQ NO: 6 or a functional fragment thereof.

Embodiment 64. In this embodiment, a method is provided as described in any one of embodiments 42 to 63, wherein the CRISPR effector protein is selected from the group of Cas9, Cas12a, Cas12b and Cas X.

Embodiment 65. In this embodiment, a method is provided as described in any one of embodiments 42 to 64, wherein the CRISPR effector protein is a Cas12a effector protein or a Cas12a derived effector protein selected from FnCas12a, LbCas12a, ErCas12a (MAD7®) or AsCas12a or variants thereof.

Embodiment 66. In this embodiment, a method is provided as described in any one of embodiments 42 to 65, wherein the nucleic acid sequence encoding a CRISPR effector protein is codon-optimized for expression in the plant cell.

Embodiment 67. In this embodiment, a method is provided as described in any one of embodiments 42 to 66, wherein the CRISPR effector protein comprises an amino acid sequence having at least 90% sequence identity or at least 95% sequence identity or is identical to the amino acid sequence of SEQ ID NO: 8.

Embodiment 68. In this embodiment, a method is provided as described in any one of embodiments 42 to 67, wherein the nucleotide sequence encoding the CRISPR effector protein comprises a nucleotide sequence having at least 90% sequence identity or at least 95% sequence identity or is identical to the nucleotide sequence of SEQ ID NO: 7.

Embodiment 69. In this embodiment, a method is provided as described in any one of embodiments 42 to 68, wherein the heterologous second promoter is a Polymerase III DNA dependent RNA polymerase dependent promotor (POLIII promoter).

Embodiment 70. In this embodiment, a method is provided as described in embodiment 69, wherein the POL III promoter is selected from the group consisting of a U6 promoter, an H1 promoter, a 5S promoter, an Adenovirus 2 (Ad2) VAI promoter, a tRNA promoter, a 7SK promoter, a chimeric POLIII promoter or a synthetic POLIII promoter.

Embodiment 71. In this embodiment, a method is provided as described in any one of embodiments 69 or 70, wherein the POLIII promoter comprises a nucleotide sequence selected from the nucleotide sequence of SEQ ID NO: 12 or SEQ ID NO: 13.

Embodiment 72. In this embodiment, a method is provided as described in any one of embodiments 42 to 71, wherein the plant cell is from a plant selected from a monocotyledonous species, a dicotyledonous species, an angiosperm species or a gymnosperm species.

Embodiment 73. In this embodiment, a method is provided as described in any one of embodiments 42 to 72, wherein the plant cell is from a plant selected from a corn plant, a rice plant, a sorghum plant, a wheat plant, an alfalfa plant, a barley plant, a millet plant, a rye plant, a sugarcane plant, a cotton plant, a soybean plant, a canola plant, a tomato plant, an onion plant, a cucumber plant, an Arabidopsis plant, or a potato plant.

Embodiment 74. In this embodiment, a method is provided as described in any one of embodiments 42 to 73, wherein the plant cell is a corn plant cell.

Embodiment 75. In this embodiment, a method is provided as described in any one of embodiments 42 to 74, wherein the guide RNA is a single guide RNA.

Embodiment 76. In this embodiment, a method is provided as described in any one of embodiments 42 to 75, wherein the guide RNA comprises at least two direct repeat sequences, flanking a spacer comprising a nucleotide sequence complementary to the target sequence.

Embodiment 77. In this embodiment, a method is provided as described in any one of embodiments 42 to 76, wherein said first plant is a haploid inducer.

Embodiment 78. In this embodiment, a method is provided as described in embodiment 77, wherein said embryo is haploid.

Embodiment 79. In this embodiment, a method is provided as described in embodiment 78, further comprising treating said haploid embryo or a plant obtained from said haploid embryo with a chromosome doubling agent.

Embodiment 80. In this embodiment, a method is provided as described in any one of embodiments 42 to 79, wherein said modification within the target sequence in the at least one cell of the embryo comprises:
(a) insertion of at least one nucleotide;
(b) deletion of at least one nucleotide;
(c) substitution of at least one nucleotide.

Embodiment 81. In yet another aspect, a method is provided of generating two or more progeny plants with unique edits from a single plant cell, the method comprising:
(a) introducing into the plant cell:
   (i) a first nucleic acid sequence encoding a CRISPR effector protein operably linked to a heterologous first promoter selected from the group consisting of a plant constitutive promoter from the Zea mays Tubulin1 gene (Zm.Tubg1), a plant constitutive promoter from the Setaria italica Ubiquitin 1 gene (SETit.Ubq1), a plant constitutive promoter from the Saccharum officinarum Ubiquitin 4 gene (So.Ubg4), a plant constitutive promoter from the Oryza sativa Actin1 gene (Os.Act) and a plant constitutive promoter from the Saccharum officinarum Ubiquitin 9 gene (So.Ubg9); and
   (ii) a second nucleic acid sequence encoding at least one guide nucleic acid operably linked to a heterologous second promoter, wherein the at least one guide nucleic acid is capable of hybridizing to a target sequence within the genome; and
(b) regenerating a first plant from the plant cell of step (a), wherein the CRISPR effector protein and at least one guide nucleic acid form a ribonucleoprotein within at least one cell of the first plant, and wherein the ribonucleoprotein generates at least one double-stranded break within the target sequence in the at least cell;
(c) pollinating the first plant of step (b);
(d) germinating two or more seeds produced from step (c) to produce two or more progeny plants with unique edits.

Embodiment 82. In this embodiment, a method is provided as described in embodiment 81, wherein the heterologous first constitutive promoter is selected from the group consisting of a plant constitutive promoter from the Setaria italica Ubiquitin 1 gene (SETit.Ubq1), a plant constitutive promoter from the Saccharum officinarum Ubiquitin 4 gene (So.Ubg4), a plant constitutive promoter from the Oryza sativa Actin1 gene (Os.Act) and a plant constitutive promoter from the Saccharum officinarum Ubiquitin 9 gene (So.Ubg9).

Embodiment 83. In this embodiment, a method is provided as described in embodiment 81, wherein the heterologous first constitutive promoter is selected from the group consisting of a plant constitutive promoter from the Setaria italica Ubiquitin 1 gene (SETit.Ubq1), a plant constitutive promoter from the Saccharum officinarum Ubiquitin 4 gene (So.Ubg4), and a plant constitutive promoter from the Saccharum officinarum Ubiquitin 9 gene (So.Ubg9).

Embodiment 84. In this embodiment, a method is provided as described in embodiment 81, wherein the heterologous first constitutive promoter is a plant constitutive promoter from the Setaria italica Ubiquitin 1 gene (SETit.Ubq1).

Embodiment 85. In this embodiment, a method is provided as described in embodiment 81, wherein the heterologous first constitutive promoter is a plant constitutive promoter from the Saccharum officinarum Ubiquitin 4 gene (So.Ubq4).

Embodiment 86. In this embodiment, a method is provided as described in embodiment 81, wherein the heterologous first constitutive promoter is a plant constitutive promoter from the Saccharum officinarum Ubiquitin 9 gene (So.Ubq9).

Embodiment 87. In this embodiment, a method is provided as described in embodiment 81, wherein the heterologous first constitutive promoter comprises a nucleic acid sequence that is at least 85% identical to a nucleic acid sequence selected from the group consisting of SEQ ID NOs:2-6 or a functional fragment thereof.

Embodiment 88. In this embodiment, a method is provided as described in embodiment 81, wherein the heterologous first constitutive promoter comprises a nucleic acid sequence that is at least 90% identical to a nucleic acid sequence selected from the group consisting of SEQ ID NOs:2-6 or a functional fragment thereof.

Embodiment 89. In this embodiment, a method is provided as described in embodiment 81, wherein the heterologous first constitutive promoter comprises a nucleic acid sequence that is at least 95% identical to a nucleic acid sequence selected from the group consisting of SEQ ID NOs:2-6 or a functional fragment thereof.

Embodiment 90. In this embodiment, a method is provided as described in embodiment 81, wherein the heterologous first constitutive promoter comprises a nucleic acid sequence that is at least 98% identical to a nucleic acid sequence selected from the group consisting of SEQ ID NOs:2-6 or a functional fragment thereof.

Embodiment 91. In this embodiment, a method is provided as described in embodiment 81, wherein the heterologous first constitutive promoter comprises a nucleic acid sequence that is at least 99% identical to a nucleic acid sequence selected from the group consisting of SEQ ID NOs:2-6 or a functional fragment thereof.

Embodiment 92. In this embodiment, a method is provided as described in embodiment 81, wherein the heterologous first constitutive promoter comprises a nucleic acid sequence that is 100% identical to a nucleic acid sequence selected from the group consisting of SEQ ID NOs:2-6 or a functional fragment thereof.

Embodiment 93. In this embodiment, a method is provided as described in any one of embodiments 87 to 92, wherein the plant constitutive promoter from the Zea mays Tubulin1 gene (Zm.Tubg1) gene comprises a nucleic acid sequence that is at least 95% identical to a nucleic acid sequence of SEQ NO: 2 or a functional fragment thereof.

Embodiment 94. In this embodiment, a method is provided as described in any one of embodiments 87 to 92, wherein the plant constitutive promoter from the Setaria italica Ubiquitin 1 gene comprises a nucleic acid sequence that is at least 95% identical to a nucleic acid sequence of SEQ NO: 3 or a functional fragment thereof.

Embodiment 95. In this embodiment, a method is provided as described in any one of embodiments 87 to 92, wherein the plant constitutive promoter from the Saccharum officinarum Ubiquitin 4 gene (So.Ubg4) comprises a nucleic acid sequence that is at least 95% identical to a nucleic acid sequence of SEQ NO: 4 or a functional fragment thereof.

Embodiment 96. In this embodiment, a method is provided as described in any one of embodiments 87 to 92, wherein the plant constitutive promoter from Oryza sativa Actin1 gene (Os.Act) the comprises a nucleic acid sequence that is at least 95% identical to a nucleic acid sequence of SEQ NO: 5 or a functional fragment thereof.

Embodiment 97. In this embodiment, a method is provided as described in any one of embodiments 87 to 92, wherein the plant constitutive promoter from the Saccharum officinarum Ubiquitin 9 gene (So.Ubg9) comprises a nucleic acid sequence that is at least 95% identical to a nucleic acid sequence of SEQ NO: 6 or a functional fragment thereof.

Embodiment 98. In this embodiment, a method is provided as described in any one of embodiments 87 to 92, wherein the plant constitutive promoter from the Zea mays Tubulin1 gene (Zm.Tubg1) gene comprises a nucleic acid sequence that is 100% identical to a nucleic acid sequence of SEQ NO: 2 or a functional fragment thereof.

Embodiment 99. In this embodiment, a method is provided as described in any one of embodiments 87 to 92, wherein the plant constitutive promoter from the Setaria italica Ubiquitin 1 gene comprises a nucleic acid sequence that is 100% identical to a nucleic acid sequence of SEQ NO: 3 or a functional fragment thereof.

Embodiment 100. In this embodiment, a method is provided as described in any one of embodiments 87 to 92, wherein the plant constitutive promoter from the Saccharum officinarum Ubiquitin 4 gene (So.Ubg4) comprises a nucleic acid sequence that is 100% identical to a nucleic acid sequence of SEQ NO: 4 or a functional fragment thereof.

Embodiment 101. In this embodiment, a method is provided as described in any one of embodiments 87 to 92, wherein the plant constitutive promoter from Oryza sativa Actin1 gene (Os.Act) the comprises a nucleic acid sequence that is 100% identical to a nucleic acid sequence of SEQ NO: 5 or a functional fragment thereof.

Embodiment 102. In this embodiment, a method is provided as described in any one of embodiments 87 to 92, wherein the plant constitutive promoter from the Saccharum officinarum Ubiquitin 9 gene (So.Ubg9) comprises a nucleic acid sequence that is at 100% identical to a nucleic acid sequence of SEQ NO: 6 or a functional fragment thereof.

Embodiment 103. In this embodiment, a method is provided as described in any one of embodiments 81 to 102, wherein the CRISPR effector protein is selected from the group of Cas9, Cas12a, Cas12b and Cas X.

Embodiment 104. In this embodiment, a method is provided as described in any one of embodiments 81 to 103, wherein the CRISPR effector protein is a Cas12a effector protein or a Cas12a derived effector protein selected from FnCas12a, LbCas12a, ErCas12a (MAD7) or AsCas12a or variants thereof.

Embodiment 105. In this embodiment, a method is provided as described in any one of embodiments 81 to 104, wherein the nucleic acid sequence encoding a CRISPR effector protein is codon-optimized for expression in the plant cell.

Embodiment 106. In this embodiment, a method is provided as described in any one of embodiments 81 to 105, wherein the CRISPR effector protein comprises an amino acid sequence having at least 90% sequence identity or at least 95% sequence identity or is identical to the amino acid sequence of SEQ ID NO: 8.

Embodiment 107. In this embodiment, a method is provided as described in any one of embodiments 81 to 106, wherein the nucleotide sequence encoding the CRISPR effector protein comprises a nucleotide sequence having at least 90% sequence identity or at least 95% sequence identity or is identical to the nucleotide sequence of SEQ ID NO: 7.

Embodiment 108. In this embodiment, a method is provided as described in any one of embodiments 81 to 107, wherein heterologous second promoter is a Polymerase III DNA dependent RNA polymerase dependent promotor (POLIII promoter).

Embodiment 109. In this embodiment, a method is provided as described in embodiment 108, wherein the POL III promoter is selected from the group consisting of a U6 promoter, an H1 promoter, a 5S promoter, an Adenovirus 2 (Ad2) VAI promoter, a tRNA promoter, a 7SK promoter, a chimeric POLIII promoter or a synthetic POLIII promoter.

Embodiment 110. In this embodiment, a method is provided as described in any one of embodiments 108 or 109, wherein the POLIII promoter comprises a nucleotide sequence selected from the nucleotide sequence of SEQ ID NO: 12 or SEQ ID NO: 13.

Embodiment 111. In this embodiment, a method is provided as described in any one of embodiments 81 to 110, wherein the plant cell is from a plant selected from a monocotyledonous species, a dicotyledonous species, an angiosperm species or a gymnosperm species.

Embodiment 112. In this embodiment, a method is provided as described in any one of embodiments 81 to 111, wherein the plant cell is from a plant selected from a corn plant, a rice plant, a sorghum plant, a wheat plant, an alfalfa plant, a barley plant, a millet plant, a rye plant, a sugarcane plant, a cotton plant, a soybean plant, a canola plant, a tomato plant, an onion plant, a cucumber plant, an Arabidopsis plant, or a potato plant.

Embodiment 113. In this embodiment, a method is provided as described in any one of embodiments 81 to 112, wherein the plant cell is a corn plant cell.

Embodiment 114. In this embodiment, a method is provided as described in any one of embodiments 81 to 113, wherein the guide RNA is a single guide RNA.

Embodiment 115. In this embodiment, a method is provided as described in any one of embodiments 81 to 114, wherein the guide RNA comprises at least two direct repeat sequences, flanking a spacer comprising a nucleotide sequence complementary to the target sequence.

Embodiment 116. In this embodiment, a method is provided as described in any one of embodiments 81 to 115, wherein said first plant is a haploid inducer.

Embodiment 117. In yet another aspect, a recombinant DNA is provided comprising nucleotide sequence encoding a CRISPR effector protein operably linked to a heterologous first promoter selected from the group consisting of a plant constitutive promoter from the Zea mays Tubulin1 gene (Zm.Tubg1), a plant constitutive promoter from the Setaria italica Ubiquitin 1 gene (SETit.Ubq1), and a plant constitutive promoter from the Saccharum officinarum Ubiquitin 9 gene (So.Ubg9).

Embodiment 118. In this embodiment, a recombinant DNA is provided as described in embodiment 117, wherein the heterologous first constitutive promoter is selected from the group consisting of a plant constitutive promoter from the Setaria italica Ubiquitin 1 gene (SETit.Ubq1), and a plant constitutive promoter from the Saccharum officinarum Ubiquitin 9 gene (So.Ubg9).

Embodiment 119. In this embodiment, a recombinant DNA is provided as described in embodiment 117, wherein the heterologous first constitutive promoter is selected from the group consisting of a plant constitutive promoter from the Setaria italica Ubiquitin 1 gene (SETit.Ubq1), and a plant constitutive promoter from the Saccharum officinarum Ubiquitin 9 gene (So.Ubg9).

Embodiment 120. In this embodiment, a recombinant DNA is provided as described in embodiment 117, wherein the heterologous first constitutive promoter is a plant constitutive promoter from the Setaria italica Ubiquitin 1 gene (SETit.Ubq1).

Embodiment 121. In this embodiment, a recombinant DNA is provided as described in embodiment 117, wherein the heterologous first constitutive promoter is a plant constitutive promoter from the Setaria italica Ubiquitin 1 gene (SETit.Ubq1).

Embodiment 122. In this embodiment, a recombinant DNA is provided as described in embodiment 117, wherein the heterologous first constitutive promoter is a plant constitutive promoter from the Saccharum officinarum Ubiquitin 9 gene (So.Ubg9).

Embodiment 123. In this embodiment, a recombinant DNA is provided as described in embodiment 117, wherein the heterologous first constitutive promoter comprises a nucleic acid sequence that is at least 85% identical to a nucleic acid sequence selected from the group consisting of SEQ ID NOs:2-3 and 6 or a functional fragment thereof.

Embodiment 124. In this embodiment, a recombinant DNA is provided as described in embodiment 117, wherein the heterologous first constitutive promoter comprises a nucleic acid sequence that is at least 90% identical to a nucleic acid sequence selected from the group consisting of SEQ ID NOs:2-3 and 6 or a functional fragment thereof.

Embodiment 125. In this embodiment, a recombinant DNA is provided as described in embodiment 117, wherein the heterologous first constitutive promoter comprises a nucleic acid sequence that is at least 95% identical to a nucleic acid sequence selected from the group consisting of SEQ ID NOs:2-3 and 6 or a functional fragment thereof.

Embodiment 126. In this embodiment, a recombinant DNA is provided as described in embodiment 117, wherein the heterologous first constitutive promoter comprises a nucleic acid sequence that is at least 98% identical to a nucleic acid sequence selected from the group consisting of SEQ ID NOs:2-3 and 6 or a functional fragment thereof.

Embodiment 127. In this embodiment, a recombinant DNA is provided as described in embodiment 117, wherein the heterologous first constitutive promoter comprises a nucleic acid sequence that is at least 99% identical to a nucleic acid sequence selected from the group consisting of SEQ ID NOs:2-3 and 6 or a functional fragment thereof.

Embodiment 128. In this embodiment, a recombinant DNA is provided as described in embodiment 117, wherein the heterologous first constitutive promoter comprises a nucleic acid sequence that is 100% identical to a nucleic acid sequence selected from the group consisting of SEQ ID NOs:2-3 and 6 or a functional fragment thereof.

Embodiment 129. In this embodiment, a recombinant DNA is provided as described in any one of embodiments 123 to 128, wherein the plant constitutive promoter from the Zea mays Tubulin1 gene (Zm.Tubg1) gene comprises a nucleic acid sequence that is at least 95% identical to a nucleic acid sequence of SEQ NO: 2 or a functional fragment thereof.

Embodiment 130. In this embodiment, a recombinant DNA is provided as described in any one of embodiments 123 to 128, wherein the plant constitutive promoter from the Setaria italica Ubiquitin 1 gene comprises a nucleic acid sequence that is at least 95% identical to a nucleic acid sequence of SEQ NO: 3 or a functional fragment thereof.

Embodiment 131. In this embodiment, a recombinant DNA is provided as described in any one of embodiments 123 to 128, wherein the plant constitutive promoter from the Setaria italica Ubiquitin 1 gene comprises a nucleic acid sequence that is at least 95% identical to a nucleic acid sequence of SEQ NO: 3 or a functional fragment thereof.

Embodiment 132. In this embodiment, a recombinant DNA is provided as described in any one of embodiments 123 to 128, Setaria italica Ubiquitin 1 gene comprises a nucleic acid sequence that is at least 95% identical to a nucleic acid sequence of SEQ NO: 3 or a functional fragment thereof.

Embodiment 133. In this embodiment, a recombinant DNA is provided as described in any one of embodiments 123 to 128, wherein the plant constitutive promoter from the Saccharum officinarum Ubiquitin 9 gene (So.Ubg9) comprises a nucleic acid sequence that is at least 95% identical to a nucleic acid sequence of SEQ NO: 6 or a functional fragment thereof.

Embodiment 134. In this embodiment, a recombinant DNA is provided as described in any one of embodiments 123 to 128, wherein the plant constitutive promoter from the Zea mays Tubulin1 gene (Zm.Tubg1) gene comprises a nucleic acid sequence that is 100% identical to a nucleic acid sequence of SEQ NO: 2 or a functional fragment thereof.

Embodiment 135. In this embodiment, a recombinant DNA is provided as described in any one of embodiments 123 to 128, wherein the plant constitutive promoter from the Setaria italica Ubiquitin 1 gene comprises a nucleic acid sequence that is 100% identical to a nucleic acid sequence of SEQ NO: 3 or a functional fragment thereof.

Embodiment 136. In this embodiment, a recombinant DNA is provided as described in any one of embodiments 123 to 128, wherein the plant constitutive promoter from the Setaria italica Ubiquitin 1 gene comprises a nucleic acid sequence that is 100% identical to a nucleic acid sequence of SEQ NO: 3 or a functional fragment thereof.

Embodiment 137. In this embodiment, a recombinant DNA is provided as described in any one of embodiments 123 to 128, wherein the plant constitutive promoter from the Setaria italica Ubiquitin 1 gene comprises a nucleic acid sequence that is 100% identical to a nucleic acid sequence of SEQ NO: 3 or a functional fragment thereof.

Embodiment 138. In this embodiment, a recombinant DNA is provided as described in any one of embodiments 123 to 128, wherein the plant constitutive promoter from the Saccharum officinarum Ubiquitin 9 gene (So.Ubg9) comprises a nucleic acid sequence that is at 100% identical to a nucleic acid sequence of SEQ NO: 6 or a functional fragment thereof.

Embodiment 139. In this embodiment, a recombinant DNA is provided as described in any one of embodiments 117 to 138, wherein the CRISPR effector protein is selected from the group of Cas9, Cas12a, Cas12b and Cas X.

Embodiment 140. In this embodiment, a recombinant DNA is provided as described in any one of embodiments 117 to 139, wherein the CRISPR effector protein is a Cas12a effector protein or a Cas12a derived effector protein selected from FnCas12a, LbCas12a, ErCas12a (MAD7) or AsCas12a or variants thereof.

Embodiment 141. In this embodiment, a recombinant DNA is provided as described in any one of embodiments 117 to 140, wherein the nucleic acid sequence encoding a CRISPR effector protein is codon-optimized for expression in the plant cell.

Embodiment 142. In this embodiment, a recombinant DNA is provided as described in any one of embodiments 117 to 141, wherein the CRISPR effector protein comprises an amino acid sequence having at least 90% sequence identity or at least 95% sequence identity or is identical to the amino acid sequence of SEQ ID NO: 8.

Embodiment 143. In this embodiment, a recombinant DNA is provided as described in any one of embodiments 117 to 142, wherein the nucleotide sequence encoding the CRISPR effector protein comprises a nucleotide sequence having at least 90% sequence identity or at least 95% sequence identity or is identical to the nucleotide sequence of SEQ ID NO: 7.

Embodiment 144. In this embodiment, a recombinant DNA is provided as described in any one of embodiments 117 to 143, further comprising a second nucleic acid sequence encoding at least one guide nucleic acid operably linked to a heterologous second promoter, wherein the at least one guide nucleic acid is capable of hybridizing to a target sequence within the genome.

Embodiment 145. In this embodiment, a recombinant DNA is provided as described in embodiment 144, wherein the heterologous second promoter is a Polymerase III DNA dependent RNA polymerase dependent promotor (POLIII promoter).

Embodiment 146. In this embodiment, a recombinant DNA is provided as described in embodiment 145, wherein the POL III promoter is selected from the group consisting of a U6 promoter, an H1 promoter, a 5S promoter, an Adenovirus 2 (Ad2) VAI promoter, a tRNA promoter, a 7SK promoter, a chimeric POLIII promoter or a synthetic POLIII promoter.

Embodiment 147. In this embodiment, a recombinant DNA is provided as described in any one of embodiments 145 or 146, wherein the POLIII promoter comprises a nucleotide sequence selected from the nucleotide sequence of SEQ ID NO: 12 or SEQ ID NO: 13.

Embodiment 148. In yet another aspect, a plant is provided comprising a recombinant DNA according to any one of embodiments 117 to 142.

Embodiment 149. In this embodiment, a plant is provided as described in embodiment 148, further comprising a second nucleic acid sequence encoding at least one guide nucleic acid operably linked to a heterologous second promoter, wherein the at least one guide nucleic acid is capable of hybridizing to a target sequence within the genome.

Embodiment 150. In still another aspect, a plant is provided comprising a recombinant DNA as described in any one of embodiments 143 to 147.

Embodiment 151. Also provided is a plant obtained by the methods as described in any one of embodiments 1 to 41.

Embodiment 152. Further provided is a plant obtained by the methods as described in any one of embodiments 42 to 80.

Embodiment 153. In yet another aspect, a plant is provided obtained by the methods as described in any one of embodiments 81 to 116.

Embodiment 154. The plant as described in any one of embodiments 148 to 153, may be plant selected from a monocotyledonous species, a dicotyledonous species, an angiosperm species or a gymnosperm species.

Embodiment 155. The plant as described in any one of embodiments 148 to 153, may be a plant selected from a corn plant, a rice plant, a sorghum plant, a wheat plant, an alfalfa plant, a barley plant, a millet plant, a rye plant, a sugarcane plant, a cotton plant, a soybean plant, a canola plant, a tomato plant, an onion plant, a cucumber plant, an Arabidopsis plant, or a potato plant.

Embodiment 156. The plant as described in any one of embodiments 148 to 153, may be a corn plant.

Embodiment 157. In another aspect, a recombinant DNA is provided comprising a plant constitutive promoter from the Zea mays Tubulin1 gene (Zm.Tubg1) gene operably linked to a heterologous nucleic acid.

Embodiment 158. In this embodiment, a recombinant DNA is provided as described in embodiment 157, wherein the heterologous nucleic acid encodes an RNA molecule.

Embodiment 159. In this embodiment, a recombinant DNA is provided as described in embodiments 157-158, wherein the heterologous nucleic acid encodes a protein.

Embodiment 160. In this embodiment, a recombinant DNA is provided as described in embodiment 158, wherein the RNA molecule is selected from the group consisting of a guide RNA, a miRNA, a double stranded RNA, a sense RNA, an antisense RNA, a ribozyme or another biologically active RNA.

Embodiment 161. In this embodiment, a recombinant DNA is provided as described in any one of embodiments 157 to 160, wherein the ZmTubg1 promoter comprises a nucleotide sequence having at least 90%, at least 91%, at least 92%, at least 93%, at least 94%, at least 95%, at least 96%, at least 97% at least 98%, at least 99% sequence identity, or is identical with the nucleotide sequence of SEQ ID No:2 or a functional fragment thereof.

Embodiment 162. In another aspect, a method is provided for expressing an RNA molecule or protein in plant comprising introducing, such as by transformation or introgressing, a recombinant DNA as described in any one of embodiments 157 to 161.

Embodiment 163. In yet another aspect, a plant, a plant part, a plant cell or commodity product obtained from such plant, plant part, or plant cell comprising a recombinant DNA as described in any one of embodiments 157 to 161.

### BRIEF DESCRIPTION OF THE SEQUENCES IN THE SEQUENCE LISTING

SEQ ID NO 1: nucleotide sequence of the Zea mays promoter, leader and intronregion of ubiquitin gene M1 (Zm.Ubq1).

SEQ ID NO 2: nucleotide sequence of the Zea mays promoter and intron region of tubulin gene 1 (Zm.Tubg1).

SEQ ID NO 3: nucleotide sequence of the Setaria italica promoter, leader and intron region of Ubitquitin 1 gene (SETit.Ubq1).

SEQ ID NO 4: nucleotide sequence of the Saccharum officinarum promoter, leader and intron region of Ubitquitin 4 gene (So.Ubg4).

SEQ ID NO 5: nucleotide sequence of the Oryza sativa promoter, leader and intron region of Actin 1 gene (Os.Act).

SEQ ID NO 6: nucleotide sequence of the Saccharum officinarum promoter, leader and intron region of Ubitquitin 9 gene (So.Ubg9).

SEQ ID NO 7: nucleotide sequence of a plant codon optimized sequence for Lachnospiraceae bacterium Cas12a RNA-guided endonuclease.

SEQ ID NO 8: amino acid sequence of Lachnospiraceae bacterium Cas12a RNA-guided endonuclease.

SEQ ID NO 9: nucleotide sequence encoding a nuclear localization signal.

SEQ ID NO 10: nucleotide sequence encoding a nuclear localization signal.

SEQ ID NO 11: nucleotide sequence of a transcription terminator sequence from a rice Lipid transfer protein (LTP) gene.

SEQ ID NO 12: nucleotide sequence of GSP2622, a synthetic Pol III promoter.

SEQ ID NO 13: nucleotide sequence of GSP2273, a synthetic Pol III promoter.

SEQ ID NO 14: nucleotide sequence of Zea mays target site TS1.

SEQ ID NO 15: nucleotide sequence of Zea mays target site TS2.

SEQ ID NO 16: nucleotide sequence of Zea mays target site TS3.

SEQ ID NO 17: nucleotide sequence of Zea mays target site TS4.

SEQ ID NO 18: nucleotide sequence of Zea mays target site TS5.

SEQ ID NO 19: nucleotide sequence of Zea mays target site TS6.

SEQ ID NO 20: nucleotide sequence of Zea mays target site TS7.

SEQ ID NO 21: nucleotide sequence of Zea mays target site TS8.

SEQ ID NO 22: nucleotide sequence of spacer region SP1 targeting TS1.

SEQ ID NO 23: nucleotide sequence of spacer region SP2 targeting TS2.

SEQ ID NO 24: nucleotide sequence of spacer region SP3 targeting TS3.

SEQ ID NO 25: nucleotide sequence of spacer region SP4 targeting TS4.

SEQ ID NO 26: nucleotide sequence of spacer region SP5 targeting TS5.

SEQ ID NO 27: nucleotide sequence of spacer region SP6 targeting TS6.

SEQ ID NO 28: nucleotide sequence of spacer region SP7 targeting TS7.

SEQ ID NO 29: nucleotide sequence of spacer region SP8 targeting TS8.

SEQ ID NO 30: nucleotide sequence of a Cas12a-compatible direct repeat included in the guide RNA of the examples.

SEQ ID NO 31: nucleotide sequence of Zea mays target site TS9.

SEQ ID NO 22: nucleotide sequence of Zea mays target site TS10.

SEQ ID NO 33: nucleotide sequence of GSP2269, a synthetic Pol III promoter.

SEQ ID NO 34: nucleotide sequence of spacer region SP9 targeting TS9.

SEQ ID NO 35: nucleotide sequence of spacer region SP10 targeting TS10.

SEQ ID NO 36: nucleotide sequence of spacer region SP11 targeting TS10.

SEQ ID NO 37: nucleotide sequence of spacer region SP12 targeting TS10.

SEQ ID NO 38: nucleotide sequence of spacer region SP13 targeting TS10.

SEQ ID NO 39: nucleotide sequence of spacer region SP14 targeting TS10.

SEQ ID NO 40: nucleotide sequence of spacer region SP15 targeting TS10.

SEQ ID NO 41: nucleotide sequence of spacer region SP16 targeting TS10.

SEQ ID NO 42: nucleotide sequence of spacer region SP17 targeting TS10.

### BRIEF DESCRIPTION OF THE DRAWINGS

FIG. 1. Graphical representation of various types of genomic edits in R1 plants obtained by selfing of R0 plants comprising a DNA construct comprising LbCas12a expressed under control of Zm.Ubq1 promoter, and further comprising guideRNA targeting TS9 (panel A) or targeting TS10 (panel B), expressed as percentage of plants with different allele types.

FIG. 2A. Schematic representation of the various recombinant DNA constructs expressing LbCas12a coding region operably linked to various constitutive promoters. Os.Act: promotor of Oryza sativa Actin gene; CP4: glyphosate tolerance marker gene; GSP2262: synthetic POL III promoter; [symbol] LbCas12a compatible direct repeat; ⋄: spacer sequence; GSP2273: synthetic POL III promoter; Zm.Tubg1: promoter of Zea mays Tubulin1 gene; SETit.Ubq1; promoter of Setaria italica Ubiquitin 1 gene; gene; So.Ubq4: promoter of Saccharum officinarum Ubiquitin 4 gene; So.Ubg9: promoter of Saccharum officinarum Ubiquitin 9 gene; Os.Act; promoter of Oryza sativa Actin1 gene; Os.TubA; promoter of Oryza sativa TubulinA gene; Lb.CAs12a: plant codon optimized sequence for Lachnospiraceae bacterium Cas12a RNA-guided endonuclease. FIG. 2B. schematic representation of the sequence of the spacer sequence in the guideRNA expression constructs under control of respectively the GSP2262 and GSP2273 POL III promoter.

FIG. 3. Graphical representation of the protein expression data (dry weight ppm) of CP4 (panel A) and Cas12a, normalized versus CP4 expression in the respective event (panel B) in R0 events comprising the various Cas12a constructs under control of the various promoters.

FIG. 4. Graphical representation of editing efficiency, analyzed by guideRNA targeting different sites in the genome, and analyzed by the LbCas12a constructs under control of the various constitutive promoters, in R0 generation (panel A) and R1 generation (panel B), expressed a percentage of edited plants.

FIG. 5. Graphical representation of editing efficiency in individual R1 events, analyzed by guideRNA targeting different sites in the genome, and analyzed by the LbCas12a constructs under the control of the various constitutive promoters, expressed as percentage of edited plants.

FIG. 6. Graphical representation of vertical transgenerational editing in singular R1 edits, obtained from R0 plants transformed with the LbCas12a constructs under the control of various constitutive promoters (panels A-E), expressed as percentage of edited plants with new edits, parental edits or wild-type alleles.

FIG. 7. Graphical representation of horizontal transgenerational editing at the TS3 target site in singular F1 events obtained by crossing R0 plants transformed with the LbCas12a constructs under the control of various constitutive promoters with tester lines not comprising such editing constructs. The efficiency is expressed as percentage of plants with edits in the genome of the tester line.

### DETAILED DESCRIPTION OF THE INVENTION

Unless defined otherwise, all technical and scientific terms used have the same meaning as commonly understood by one of ordinary skill in the art to which this disclosure belongs. Where a term is provided in the singular, the inventors also contemplate aspects of the disclosure described by the plural of that term. Where there are discrepancies in terms and definitions used in references that are incorporated by reference, the terms used in this application shall have the definitions given herein. Other technical terms used have their ordinary meaning in the art in which they are used, as exemplified by various art-specific dictionaries, for example, “The American Heritage® Science Dictionary” (Editors of the American Heritage Dictionaries, 2011, Houghton Mifflin Harcourt, Boston and New York), the “McGraw-Hill Dictionary of Scientific and Technical Terms” (6th edition, 2002, McGraw-Hill, New York), or the “Oxford Dictionary of Biology” (6th edition, 2008, Oxford University Press, Oxford and New York). The inventors do not intend to be limited to a mechanism or mode of action. Reference thereto is provided for illustrative purposes only.

The practice of this disclosure includes, unless otherwise indicated, conventional techniques of biochemistry, chemistry, molecular biology, microbiology, cell biology, plant biology, genomics, biotechnology, and genetics, which are within the skill of the art. See, for example, Green and Sambrook, Molecular Cloning: A Laboratory Manual, 4th edition (2012); Current Protocols In Molecular Biology (F. M. Ausubel, et al. eds., (1987)); Plant Breeding Methodology (N. F. Jensen, Wiley-Interscience (1988)); the series Methods In Enzymology (Academic Press, Inc.): PCR 2: A Practical Approach (M. J. MacPherson, B. D. Hames and G. R. Taylor eds. (1995)); Harlow and Lane, eds. (1988) Antibodies, A Laboratory Manual; Animal Cell Culture (R. I. Freshney, ed. (1987)); Recombinant Protein Purification: Principles And Methods, 18-1142-75, GE Healthcare Life Sciences; C. N. Stewart, A. Touraev, V. Citovsky, T. Tzfira eds. (2011) Plant Transformation Technologies (Wiley-Blackwell); and R. H. Smith (2013) Plant Tissue Culture: Techniques and Experiments (Academic Press, Inc.).

Any references cited herein, including, e.g., all patents, published patent applications, and non-patent publications, are incorporated herein by reference in their entirety.

When a grouping of alternatives is presented, any and all combinations of the members that make up that grouping of alternatives is specifically envisioned. For example, if an item is selected from a group consisting of A, B, C, and D, the inventors specifically envision each alternative individually (e.g., A alone, B alone, etc.), as well as combinations such as A, B, and D; A and C; B and C; etc.

As used herein, terms in the singular and the singular forms “a,” “an,” and “the,” for example, include plural referents unless the content clearly dictates otherwise.

Any composition, nucleic acid molecule, polypeptide, cell, plant, etc. provided herein is specifically envisioned for use with any method provided herein.

The current disclosure relates to the use of selected constitutive promoters to express CRISPR effector proteins for vertical or horizontal transgenerational editing in plants. In one aspect, a recombinant DNA is provided comprising nucleotide sequence encoding a CRISPR effector protein operably linked to a heterologous first promoter selected from the group consisting of a plant constitutive promoter from the Zea mays Tubulin1 gene (Zm.Tubg1), a plant constitutive promoter from the Setaria italica Ubiquitin 1 gene (SETit.Ubq1), a plant constitutive promoter from the Saccharum officinarum Ubiquitin 4 gene (So.Ubg4), a plant constitutive promoter from the Oryza sativa Actin1 gene (Os.Act) and a plant constitutive promoter from the Saccharum officinarum Ubiquitin 9 gene (So.Ubg9), particularly a recombinant DNA is provided comprising nucleotide sequence encoding a CRISPR effector protein operably linked to a heterologous first promoter selected from the group consisting of a plant constitutive promoter from the Setaria italica Ubiquitin 1 gene (SETit.Ubq1), a plant constitutive promoter from the Saccharum officinarum Ubiquitin 4 gene (So.Ubg4) and a plant constitutive promoter from the Saccharum officinarum Ubiquitin 9 gene (So.Ubq9).

As used herein, a Zm.Tubg1 promoter is a plant constitutive promoter from the Zea mays Tubulin1 gene (Zm.Tubg1) gene, and typically comprises a nucleic acid sequence that is least 85% identical, at least 90% identical, at least 95% identical, at least 98% identical, at least 99% identical or 100% identical to a nucleic acid sequence of SEQ NO: 2 or a functional fragment thereof

As used herein, a SETit.Ubq1 promoter is a plant constitutive promoter from the Setaria italica Ubiquitin 1 gene, and typically comprises a nucleic acid sequence that is least 85% identical, at least 90% identical, at least 95% identical, at least 98% identical, at least 99% identical or 100% identical to a nucleic acid sequence of SEQ NO: 3 or a functional fragment thereof.

As used herein, a So.Ubg4 promoter is a plant constitutive promoter from the Saccharum officinarum Ubiquitin 4 gene, and typically comprises a nucleic acid sequence that is least 85% identical, at least 90% identical, at least 95% identical, at least 98% identical, at least 99% identical or 100% identical to a nucleic acid sequence of SEQ NO: 4 or a functional fragment thereof.

As used herein, a Os.Act promoter is a plant constitutive promoter from the Oryza sativa Actin1 gene, and typically comprises a nucleic acid sequence that is least 85% identical, at least 90% identical, at least 95% identical, at least 98% identical, at least 99% identical or 100% identical to a nucleic acid sequence of SEQ NO: 5 or a functional fragment thereof.

As used herein, a So.Ubg9 promoter is a plant constitutive promoter from the Saccharum officinarum Ubiquitin 9 gene, and typically comprises a nucleic acid sequence that is least 85% identical, at least 90% identical, at least 95% identical, at least 98% identical, at least 99% identical or 100% identical to a nucleic acid sequence of SEQ NO: 6 or a functional fragment thereof.

As used herein, functional fragments of the constitutive promoters disclosed herein are fragments which comprise at least about 50, at least about 75, at least about 95, at least about 100, at least about 125, at least about 150, at least about 175, at least about 200, at least about 225, at least about 250, at least about 275, at least about 300, at least about 325, at least about 350, at least about 375, at least about 400 contiguous nucleotides, at least about 425, at least about 450, at least about 475, or longer, of a DNA molecule having promoter activity as disclosed herein.

As used herein, a “promoter” is a nucleotide sequence that controls or regulates the transcription of a nucleotide sequence (e.g., a coding sequence) that is operably associated with the promoter. The coding sequence controlled or regulated by a promoter may encode a polypeptide and/or a functional RNA. A “promoter” may refer to a nucleotide sequence that contains a binding site for RNA polymerase II and directs the initiation of transcription. In general, promoters are found 5′, or upstream, relative to the start of the coding region of the corresponding coding sequence. A promoter may comprise other elements that act as regulators of gene expression; e.g., a promoter region. These include a TATA box consensus sequence, and often a CAAT box consensus sequence (Breathnach and Chambon (1981) Annu. Rev. Biochem. 50:349). In plants, the CAAT box may be substituted by the AGGA box (Messing et al., (1983) in Genetic Engineering of Plants, T. Kosuge, C. Meredith and A. Hollaender (eds.), Plenum Press, pp. 211-227).

### Other Plant-Expressible Promoters

Promoters useful with this invention, e.g. for expression of the guideRNAs, can include, constitutive, inducible, temporally regulated, developmentally regulated, chemically regulated, tissue-preferred and/or tissue-specific promoters for use in the preparation of recombinant nucleic acid molecules, e.g., “synthetic nucleic acid constructs” or “protein-RNA complex.” These various types of promoters are known in the art.

The choice of promoter may vary depending on the temporal and spatial requirements for expression, and also may vary based on the host cell to be transformed. Promoters for many different organisms are well known in the art. Based on the extensive knowledge present in the art, the appropriate promoter can be selected for the particular host organism of interest. Thus, for example, much is known about promoters upstream of highly constitutively expressed genes in model organisms and such knowledge can be readily accessed and implemented in other systems as appropriate.

In some embodiments, the promoter expressing the guideRNAs comprising the spacer sequence complementary to a target site, such as a genomic target site, as described herein, may be selected from RNA polymerase III (Pol III) promoters. In some aspects, the POL III promoter may be a U6 promoter, an H1 promoter, a 5S promoter, an Adenovirus 2 (Ad2) VAI promoter, a tRNA promoter, and a 7SK promoter. See, for example, Schramm and Hernandez, 2002, Genes & Development, 16:2593-2620, which is incorporated by reference herein in its entirety.

In some aspects, the POL III promoters may be derived from small nuclear RNA (snRNA) encoding genes. In some aspects, the POL III promoters may be selected from the corn, tomato and soybean U6, U3, U2, U5 and 7SL snRNA promoters disclosed in WO2015/131101 (incorporated herein by reference in its entirety) including the snRNA promoter sequences of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20; SEQ ID NOs: 146-149, SEQ ID NOs: 160-166, SEQ ID NOs: 201 or SEQ ID NO: 283, included therein in the accompanying sequence listing.

In some aspects, the POL III promoters may be synthetic snRNA promoters, such as the snRNA promoters described in WO2022/232407 (incorporated herein by reference in its entirety) including the snRNA promoter sequences of SEQ ID Nos: 1-10 included therein in the accompanying sequence listing.

In some aspects, the POL III promoters may be selected from POL III promoters comprising a nucleotide sequence of SEQ ID Nos 12 or 13 of the accompanying sequence listing.

In some aspects, the POL III promoters may be chimeric POL III promoters. In some aspects, the POL III promoters may be variants of the POL III promoters disclosed herein. In some aspects, a variant of a POL III promoters comprising a sequence that, when optimally aligned to the reference sequence has at least about 85 percent identity, at least about 86 percent identity, at least about 87 percent identity, at least about 88 percent identity, at least about 89 percent identity, at least about 90 percent identity, at least about 91 percent identity, at least about 92 percent identity, at least about 93 percent identity, at least about 94 percent identity, at least about 95 percent identity, at least about 96 percent identity, at least about 97 percent identity, at least about 98 percent identity, or at least about 99 percent identity to the reference sequence and having promoter activity as disclosed herein are provided. Variants of the POL III promoters may comprise a nucleotide sequence having at least about 85 percent identity, at least about 86 percent identity, at least about 87 percent identity, at least about 88 percent identity, at least about 89 percent identity, at least about 90 percent identity, at least about 91 percent identity, at least about 92 percent identity, at least about 93 percent identity, at least about 94 percent identity, at least about 95 percent identity, at least about 96 percent identity, at least about 97 percent identity, at least about 98 percent identity, or at least about 99 percent identity of a nucleotide sequence of any one of SEQ ID NOs: 12 or 13.

In some aspects, fragments of the POL III promoters disclosed herein may be used according to the invention, wherein the fragments comprise at least about 50, at least about 75, at least about 95, at least about 100, at least about 125, at least about 150, at least about 175, at least about 200, at least about 225, at least about 250, at least about 275, at least about 300, at least about 325, at least about 350, at least about 375, at least about 400 contiguous nucleotides, at least about 425, at least about 450, at least about 475, or longer, of a DNA molecule having promoter activity as disclosed herein. In certain embodiments, provided are fragments of a small nuclear RNA promoter provided herein, having gene expression activity. Fragments of a POL III promoter may comprise at least about 50, at least about 75, at least about 95, at least about 100, at least about 125, at least about 150, at least about 175, at least about 200, at least about 225, at least about 250, at least about 275, at least about 300, at least about 325, at least about 350, at least about 375, at least about 400, at least about 425, at least about 450, at least about 475, or longer contiguous nucleotides of any one of SEQ ID Nos: 10-13, 15-17 or 19-47.

### RNA Guided Nucleases

Guided nucleases are nucleases that form a complex (e.g., a ribonucleoprotein) with a guide nucleic acid molecule (e.g., a guide RNA), which then guides the complex to a target site within a target sequence. One non-limiting example of guided nucleases are CRISPR nucleases.

CRISPR (Clustered Regularly Interspaced Short Palindromic Repeats) nucleases (e.g., Cas9, CasX, Cas12a (also referred to as Cpf1), CasY, MAD7®) are proteins found in bacteria that are guided by guide RNAs (“gRNAs”) to a target nucleic acid molecule, where the endonuclease can then cleave one or two strands the target nucleic acid molecule. Although the origins of CRISPR nucleases are bacterial, many CRISPR nucleases have been shown to function in eukaryotic cells.

While not being limited by any particular scientific theory, a CRISPR nuclease forms a complex with a guide RNA (gRNA), which hybridizes with a complementary target site, thereby guiding the CRISPR nuclease to the target site. In class II CRISPR-Cas systems, CRISPR arrays, including spacers, are transcribed during encounters with recognized invasive DNA and are processed into small interfering CRISPR RNAs (crRNAs). The crRNA comprises a repeat sequence and a spacer sequence which is complementary to a specific protospacer sequence in an invading pathogen. The spacer sequence can be designed to be complementary to target sequences in a eukaryotic genome.

CRISPR nucleases associate with their respective crRNAs in their active forms. CasX, similar to the class II endonuclease Cas9, requires another non-coding RNA component, referred to as a trans-activating crRNA (tracrRNA), to have functional activity. Nucleic acid molecules provided herein can combine a crRNA and a tracrRNA into one nucleic acid molecule in what is herein referred to as a “single guide RNA” (sgRNA). Cas12a or MAD7® do not require a tracrRNA to be guided to a target site; a crRNA alone is sufficient for Cas12a or MAD7®. The gRNA guides the active CRISPR nuclease complex to a target site, where the CRISPR nuclease can cleave the target site.

When an RNA-guided CRISPR nuclease and a guide RNA form a complex, the whole system is called a “ribonucleoprotein.” Ribonucleoproteins provided herein can also comprise additional nucleic acids or proteins.

A prerequisite for cleavage of the target site by a CRISPR ribonucleoprotein is the presence of a conserved Protospacer Adjacent Motif (PAM) near the target site. Depending on the CRISPR nuclease, cleavage can occur within a certain number of nucleotides (e.g., between 18-23 nucleotides for Cas12a) from the PAM site. PAM sites are only required for type I and type II CRISPR associated proteins, and different CRISPR endonucleases recognize different PAM sites. Without being limiting, Cas12a can recognize at least the following PAM sites: TTTN, and YTN; CasX can recognize at least the following PAM sites: TTCN, TTCA, and TTC and MAD7® nuclease recognizes T-rich PAM sequences YTTN and seems to prefer TTTN to CTTN PAMs (where T is thymine; C is cytosine; A is adenine; Y is thymine or cytosine; and N is thymine, cytosine, guanine, or adenine).

Cas12a is an RNA-guided nuclease of a class II, type V CRISPR/Cas system. Cas12a nucleases generate staggered cuts when cleaving a double-stranded DNA molecule. Staggered cuts of double-stranded DNA produce a single-stranded DNA overhang of at least one nucleotide. This is in contrast to a blunt-end cut (such as those generated by Cas9), which does not produce a single-stranded DNA overhang when cutting double-stranded DNA.

In an aspect, a Cas12a nuclease provided herein is a Lachnospiraceae bacterium Cas12a (LbCas12a) nuclease. In another aspect, a Cas12a nuclease provided herein is a Francisella novicida Cas12a (FnCas12a) nuclease. In an aspect, a Cas12a nuclease is selected from the group consisting of LbCas12a and FnCas12a.

In an aspect, a Cas12a nuclease, or a nucleic acid encoding a Cas12a nuclease, is derived from a bacteria genus selected from the group consisting of Streptococcus, Campylobacter, Nitratifractor, Staphylococcus, Parvibaculum, Roseburia, Neisseria, Gluconacetobacter, Azospirillum, Sphaerochaeta, Lactobacillus, Eubacterium, Corynebacter, Carnobacterium, Rhodobacter, Listeria, Paludibacter, Clostridium, Lachnospiraceae, Clostridiaridium, Leptotrichia, Francisella, Legionella, Alicyclobacillus, Methanomethyophilus, Porphyromonas, Prevotella, Bacteroidetes, Helcococcus, Letospira, Desulfovibrio, Desulfonatronum, Opitutaceae, Tuberibacillus, Bacillus, Brevibacilus, Methylobacterium, Acidaminococcus, Peregrinibacteria, Butyrivibrio, Parcubacteria, Smithella, Candidatus, Moraxella, and Leptospira.

In an aspect, a Cas12a nuclease is encoded by a polynucleotide comprising a sequence at least 80% identical to a polynucleotide of SEQ ID NO: 7. In another aspect, a Cas12a nuclease is encoded by a polynucleotide comprising a sequence at least 85% identical to a polynucleotide of SEQ ID NO: 7. In another aspect, a Cas12a nuclease is encoded by a polynucleotide comprising a sequence at least 90% identical to a polynucleotide of SEQ ID NO: 7. In another aspect, a Cas12a nuclease is encoded by a polynucleotide comprising a sequence at least 95% identical to a polynucleotide of SEQ ID NO: 7. In another aspect, a Cas12a nuclease is encoded by a polynucleotide comprising a sequence at least 96% identical to a polynucleotide selected from the group consisting of SEQ ID NO: 7. In another aspect, a Cas12a nuclease is encoded by a polynucleotide comprising a sequence at least 97% identical to a polynucleotide of SEQ ID NO: 7. In another aspect, a Cas12a nuclease is encoded by a polynucleotide comprising a sequence at least 98% identical to a polynucleotide of SEQ ID NO: 7. In another aspect, a Cas12a nuclease is encoded by a polynucleotide comprising a sequence at least 99% identical to a polynucleotide of SEQ ID NO: 7. In another aspect, a Cas12a nuclease is encoded by a polynucleotide comprising a sequence 100% identical to a polynucleotide of SEQ ID NO: 7.

In an aspect, a Cas12a nuclease provided herein comprises an amino acid sequence having at least 80% identical to an amino acid sequence selected from SEQ ID NO: 8. In another aspect, a Cas12a nuclease provided herein comprises an amino acid sequence having at least 85% identical to an amino acid sequence selected from SEQ ID NO: 8. In another aspect, a Cas12a nuclease provided herein comprises an amino acid sequence having at least 90% identical to an amino acid sequence selected from SEQ ID NO: 8. In another aspect, a Cas12a nuclease provided herein comprises an amino acid sequence having at least 95% identical to an amino acid sequence selected from SEQ ID NO: 8. In another aspect, a Cas12a nuclease provided herein comprises an amino acid sequence having at least 96% identical to an amino acid sequence selected from SEQ ID NO: 8. In another aspect, a Cas12a nuclease provided herein comprises an amino acid sequence having at least 97% identical to an amino acid sequence selected from SEQ ID NO:8. In another aspect, a Cas12a nuclease provided herein comprises an amino acid sequence having at least 98% identical to an amino acid sequence selected from SEQ ID NO: 8. In another aspect, a Cas12a nuclease provided herein comprises an amino acid sequence having at least 99% identical to an amino acid sequence selected from SEQ ID NO:8. In another aspect, a Cas12a nuclease provided herein comprises an amino acid sequence having at 100% identity to an amino acid sequence selected from SEQ ID NO:8.

In an aspect, a Cas12a provided herein is a variant Lachnospiraceae bacterium Cas12a (LbCas12a) nuclease with enhanced DNA cleavage activities at non-canonical TTTT protospacer adjacent motifs such as described in US2021/0348144 (incorporated herein by reference in its entirety) In another aspect, a Cas12a provided herein is a variant Lachnospiraceae bacterium Cas12a (LbCas12a) nuclease with enhanced activity as described in US20230040148 (incorporated herein by reference in its entirety) such as the LbCas12a-ultra having an N527R and E795L substitution in its amino acid sequence (reference amino acid sequence is SEQ ID NO: 8).

In another aspect, a Cas12a provided herein is a variant Lachnospiraceae bacterium Cas12a (LbCas12a) nuclease as described in US20190010481 (herein incorporated by reference in its entirety) having a D156R substitution in its amino acid sequence (reference amino acid sequence is SEQ ID NO: 8)

In an aspect, a Cas12a provided herein provided herein is a variant Lachnospiraceae bacterium Cas12a (LbCas12a) nuclease recognizing a PAM variant TYCV having a G532R and K595R substitution in its amino acid sequence (reference amino acid sequence is SEQ ID NO: 8) or a variant Lachnospiraceae bacterium Cas12a (LbCas12a) nuclease recognizing a PAM variant TATT having a G532R, K538R and Y524R substitution in its amino acid sequence (reference amino acid sequence is SEQ ID NO: 8) as disclosed in WO2016205711 (herein incorporated by reference in its entirety).

CasX is a type of class II CRISPR-Cas nuclease that has been identified in the bacterial phyla Deltaproteobacteria and Planctomycetes. Similar to Cas12a, CasX nucleases generate staggered cuts when cleaving a double-stranded DNA molecule. However, unlike Cas12a, CasX nucleases require a crRNA and a tracrRNA, or a single-guide RNA, in order to target and cleave a target nucleic acid.

In an aspect, a CasX nuclease provided herein is a CasX nuclease from the phylum Deltaproteobacteria. In another aspect, a CasX nuclease provided herein is a CasX nuclease from the phylum Planctomycetes. Without being limiting, additional suitable CasX nucleases are those set forth in WO 2019/084148, which is incorporated by reference herein in its entirety.

MAD7® (also known as ErCas12a) is an engineered nuclease of the Class 2 type V-A CRISPR-Cas (Cas12a/Cpf1) family with a low level of homology to canonical Cas12a nucleases. MAD7® nucleases generate staggered cuts when cleaving a double-stranded DNA molecule. MAD7® nuclease was initially identified in Eubacterium rectale. It only requires a crRNA like canonical Cas12a. An ErCas12a/MAD7® encoding nucleotide sequence can be found in the supplementary data (sequences S1) provided with Lin et al., 2021, Journal of Genetics and Genomics 48, pages 444-451)

In an aspect, a guided nuclease capable of generating a staggered cut in a double-stranded DNA molecule is selected from the group consisting of Cas12a; MAD7® and CasX. In an aspect, a guided nuclease is selected from the group consisting of Cas12a, MAD7® and CasX.

In an aspect, a guided nuclease is a RNA-guided nuclease. In another aspect, a guided nuclease is a CRISPR nuclease. In another aspect, a guided nuclease is a Cas12a nuclease. In another aspect, a guided nuclease is a CasX nuclease. In another aspect, a guided nuclease is a MAD7® nuclease.

As used herein, a “nuclear localization signal” (NLS) refers to an amino acid sequence that “tags” a protein for import into the nucleus of a cell. In an aspect, a nucleic acid molecule provided herein encodes a nuclear localization signal. In another aspect, a nucleic acid molecule provided herein encodes two or more nuclear localization signals.

In an aspect, a Cas12a nuclease provided herein comprises a nuclear localization signal. In an aspect, a nuclear localization signal is positioned on the N-terminal end of a Cas12a nuclease. In a further aspect, a nuclear localization signal is positioned on the C-terminal end of a Cas12a nuclease. In yet another aspect, a nuclear localization signal is positioned on both the N-terminal end and the C-terminal end of a Cas12a nuclease.

In an aspect, a CasX nuclease provided herein comprises a nuclear localization signal. In an aspect, a nuclear localization signal is positioned on the N-terminal end of a CasX nuclease. In a further aspect, a nuclear localization signal is positioned on the C-terminal end of a CasX nuclease. In yet another aspect, a nuclear localization signal is positioned on both the N-terminal end and the C-terminal end of a CasX nuclease.

In an aspect, a MAD7® nuclease provided herein comprises a nuclear localization signal. In an aspect, a nuclear localization signal is positioned on the N-terminal end of a MAD7® nuclease. In a further aspect, a nuclear localization signal is positioned on the C-terminal end of a MAD7® nuclease. In yet another aspect, a nuclear localization signal is positioned on both the N-terminal end and the C-terminal end of a MAD7® nuclease

In an aspect, a ribonucleoprotein comprises at least one nuclear localization signal. In another aspect, a ribonucleoprotein comprises at least two nuclear localization signals.

Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www[dot]kazusa[dot]or[dot]jp[forwards slash]codon and these tables can be adapted in a number of ways. See Nakamura et al., 2000, Nucl. Acids Res. 28:292. Computer algorithms for codon optimizing a particular sequence for expression in a particular plant cell are also available, such as Gene Forge (Aptagen; Jacobus, PA), are also available.

As used herein, “codon optimization” refers to a process of modifying a nucleic acid sequence for enhanced expression in a plant cell of interest by replacing at least one codon (e.g., at least 1, 2, 3, 4, 5, 10, 15, 20, 25, 50, or more codons) of a sequence with codons that are more frequently or most frequently used in the genes of the plant cell while maintaining the original amino acid sequence (e.g., introducing silent mutations).

In an aspect, one or more codons (e.g., 1, 2, 3, 4, 5, 10, 15, 20, 25, 50, or more, or all codons) in a sequence encoding a guided nuclease correspond to the most frequently used codon for a particular amino acid. In another aspect, one or more codons (e.g., 1, 2, 3, 4, 5, 10, 15, 20, 25, 50, or more, or all codons) in a sequence encoding a Cas12a nuclease or a CasX nuclease or a MAD7® nuclease correspond to the most frequently used codon for a particular amino acid. As to codon usage in plants, reference is made to Campbell and Gowri, 1990, Plant Physiol., 92: 1-11; and Murray et al., 1989, Nucleic Acids Res., 17:477-98, each of which is incorporated herein by reference in their entireties.

In an aspect, a nucleic acid molecule encodes a guided nuclease that is codon optimized for a plant. In an aspect, a nucleic acid molecule encodes a Cas12a nuclease that is codon optimized for a plant. In an aspect, a nucleic acid molecule encodes a CasX nuclease that is codon optimized for a plant. In an aspect, a nucleic acid molecule encodes a MAD7® nuclease that is codon optimized for a plant

In another aspect, a nucleic acid molecule provided herein encodes a guided nuclease that is codon optimized for a plant cell. In another aspect, a nucleic acid molecule provided herein encodes a guided nuclease that is codon optimized for a monocotyledonous plant species. In another aspect, a nucleic acid molecule provided herein encodes a guided nuclease that is codon optimized for a dicotyledonous plant species. In a further aspect, a nucleic acid molecule provided herein encodes a guided nuclease that is codon optimized for a gymnosperm plant species. In a further aspect, a nucleic acid molecule provided herein encodes a guided nuclease that is codon optimized for an angiosperm plant species. In a further aspect, a nucleic acid molecule provided herein encodes a guided nuclease that is codon optimized for a corn cell. In a further aspect, a nucleic acid molecule provided herein encodes a guided nuclease that is codon optimized for a soybean cell. In a further aspect, a nucleic acid molecule provided herein encodes a guided nuclease that is codon optimized for a rice cell. In a further aspect, a nucleic acid molecule provided herein encodes a guided nuclease that is codon optimized for a wheat cell. In a further aspect, a nucleic acid molecule provided herein encodes a guided nuclease that is codon optimized for a cotton cell. In a further aspect, a nucleic acid molecule provided herein encodes a guided nuclease that is codon optimized for a sorghum cell. In a further aspect, a nucleic acid molecule provided herein encodes a guided nuclease that is codon optimized for an alfalfa cell. In a further aspect, a nucleic acid molecule provided herein encodes a guided nuclease that is codon optimized for a sugarcane cell. In a further aspect, a nucleic acid molecule provided herein encodes a guided nuclease that is codon optimized for an Arabidopsis cell. In a further aspect, a nucleic acid molecule provided herein encodes a guided nuclease that is codon optimized for a tomato cell. In a further aspect, a nucleic acid molecule provided herein encodes a guided nuclease that is codon optimized for a cucumber cell. In a further aspect, a nucleic acid molecule provided herein encodes a guided nuclease that is codon optimized for a potato cell. In a further aspect, a nucleic acid molecule provided herein encodes a guided nuclease that is codon optimized for an onion cell.

In another aspect, a nucleic acid molecule provided herein encodes a Cas12a nuclease that is codon optimized for a plant cell. In another aspect, a nucleic acid molecule provided herein encodes a Cas12a nuclease that is codon optimized for a monocotyledonous plant species. In another aspect, a nucleic acid molecule provided herein encodes a Cas12a nuclease that is codon optimized for a dicotyledonous plant species. In a further aspect, a nucleic acid molecule provided herein encodes a Cas12a nuclease that is codon optimized for a gymnosperm plant species. In a further aspect, a nucleic acid molecule provided herein encodes a Cas12a nuclease that is codon optimized for an angiosperm plant species. In a further aspect, a nucleic acid molecule provided herein encodes a Cas12a nuclease that is codon optimized for a corn cell. In a further aspect, a nucleic acid molecule provided herein encodes a Cas12a nuclease that is codon optimized for a soybean cell. In a further aspect, a nucleic acid molecule provided herein encodes a Cas12a nuclease that is codon optimized for a rice cell. In a further aspect, a nucleic acid molecule provided herein encodes a Cas12a nuclease that is codon optimized for a wheat cell. In a further aspect, a nucleic acid molecule provided herein encodes a Cas12a nuclease that is codon optimized for a cotton cell. In a further aspect, a nucleic acid molecule provided herein encodes a Cas12a nuclease that is codon optimized for a sorghum cell. In a further aspect, a nucleic acid molecule provided herein encodes a Cas12a nuclease that is codon optimized for an alfalfa cell. In a further aspect, a nucleic acid molecule provided herein encodes a Cas12a nuclease that is codon optimized for a sugar cane cell. In a further aspect, a nucleic acid molecule provided herein encodes a Cas12a nuclease that is codon optimized for an Arabidopsis cell. In a further aspect, a nucleic acid molecule provided herein encodes a Cas12a nuclease that is codon optimized for a tomato cell. In a further aspect, a nucleic acid molecule provided herein encodes a Cas12a nuclease that is codon optimized for a cucumber cell. In a further aspect, a nucleic acid molecule provided herein encodes a Cas12a nuclease that is codon optimized for a potato cell. In a further aspect, a nucleic acid molecule provided herein encodes a Cas12a nuclease that is codon optimized for an onion cell.

In another aspect, a nucleic acid molecule provided herein encodes a CasX nuclease that is codon optimized for a plant cell. In another aspect, a nucleic acid molecule provided herein encodes a CasX nuclease that is codon optimized for a monocotyledonous plant species. In another aspect, a nucleic acid molecule provided herein encodes a CasX nuclease that is codon optimized for a dicotyledonous plant species. In a further aspect, a nucleic acid molecule provided herein encodes a CasX nuclease that is codon optimized for a gymnosperm plant species. In a further aspect, a nucleic acid molecule provided herein encodes a CasX nuclease that is codon optimized for an angiosperm plant species. In a further aspect, a nucleic acid molecule provided herein encodes a CasX nuclease that is codon optimized for a corn cell. In a further aspect, a nucleic acid molecule provided herein encodes a CasX nuclease that is codon optimized for a soybean cell. In a further aspect, a nucleic acid molecule provided herein encodes a CasX nuclease that is codon optimized for a rice cell. In a further aspect, a nucleic acid molecule provided herein encodes a CasX nuclease that is codon optimized for a wheat cell. In a further aspect, a nucleic acid molecule provided herein encodes a CasX nuclease that is codon optimized for a cotton cell. In a further aspect, a nucleic acid molecule provided herein encodes a CasX nuclease that is codon optimized for a sorghum cell. In a further aspect, a nucleic acid molecule provided herein encodes a CasX nuclease that is codon optimized for an alfalfa cell. In a further aspect, a nucleic acid molecule provided herein encodes a CasX nuclease that is codon optimized for a sugar cane cell. In a further aspect, a nucleic acid molecule provided herein encodes a CasX nuclease that is codon optimized for an Arabidopsis cell. In a further aspect, a nucleic acid molecule provided herein encodes a CasX nuclease that is codon optimized for a tomato cell. In a further aspect, a nucleic acid molecule provided herein encodes a CasX nuclease that is codon optimized for a cucumber cell. In a further aspect, a nucleic acid molecule provided herein encodes a CasX nuclease that is codon optimized for a potato cell. In a further aspect, a nucleic acid molecule provided herein encodes a CasX nuclease that is codon optimized for an onion cell. In another aspect, a nucleic acid molecule provided herein encodes a MAD7® nuclease that is codon optimized for a plant cell. In another aspect, a nucleic acid molecule provided herein encodes a MAD7® nuclease that is codon optimized for a monocotyledonous plant species. In another aspect, a nucleic acid molecule provided herein encodes a MAD7® nuclease that is codon optimized for a dicotyledonous plant species. In a further aspect, a nucleic acid molecule provided herein encodes a MAD7® nuclease that is codon optimized for a gymnosperm plant species. In a further aspect, a nucleic acid molecule provided herein encodes a MAD7® nuclease that is codon optimized for an angiosperm plant species. In a further aspect, a nucleic acid molecule provided herein encodes a MAD7® nuclease that is codon optimized for a corn cell. In a further aspect, a nucleic acid molecule provided herein encodes a MAD7® nuclease that is codon optimized for a soybean cell. In a further aspect, a nucleic acid molecule provided herein encodes a MAD7® nuclease that is codon optimized for a rice cell. In a further aspect, a nucleic acid molecule provided herein encodes a MAD7® nuclease that is codon optimized for a wheat cell. In a further aspect, a nucleic acid molecule provided herein encodes a MAD7® nuclease that is codon optimized for a cotton cell. In a further aspect, a nucleic acid molecule provided herein encodes a MAD7® nuclease that is codon optimized for a sorghum cell. In a further aspect, a nucleic acid molecule provided herein encodes a MAD7® nuclease that is codon optimized for an alfalfa cell. In a further aspect, a nucleic acid molecule provided herein encodes a MAD7® nuclease that is codon optimized for a sugar cane cell. In a further aspect, a nucleic acid molecule provided herein encodes a MAD7® nuclease that is codon optimized for an Arabidopsis cell. In a further aspect, a nucleic acid molecule provided herein encodes a MAD7® nuclease that is codon optimized for a tomato cell. In a further aspect, a nucleic acid molecule provided herein encodes a MAD7® nuclease that is codon optimized for a cucumber cell. In a further aspect, a nucleic acid molecule provided herein encodes a MAD7® nuclease that is codon optimized for a potato cell. In a further aspect, a nucleic acid molecule provided herein encodes a MAD7® nuclease that is codon optimized for an onion cell.

In some aspects the guided nuclease may be selected from Cas9, C2c1, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Csn1, Csx12, Cas10, Csy1, Csy2, Csy3, Cse1, Cse2, 30 Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), Csf5 nuclease, Cas12c (C2c3), Cas12d (CasY), Cas12e (CasX), Cas12g, Cas12h, Cas12i, C2c4, C2c5, C2c8, C2c9, C2c10, Cas14a, Cas14b, Cas14c effector protein

In some aspects, the guided nuclease, such as a CRISPR/Cas effector protein useful with the invention may comprise a mutation in its nuclease active site (e.g., RuvC, HNH, e.g., RuvC site of a Cas12a nuclease domain, e.g., RuvC site and/or HNH site of a Cas9 nuclease domain). A CRISPR-Cas effector protein having a mutation in its nuclease active site, and therefore, no longer comprising nuclease activity, is commonly referred to as “dead,” e.g., dCas. In some embodiments, a CRISPR-Cas effector protein domain or polypeptide having a mutation in its nuclease active site may have impaired activity or reduced activity as compared to the same CRISPR-Cas effector protein without the mutation, e.g., a nickase, e.g., Cas9 nickase, Cas12a nickase.

In some aspects, the guided nuclease may comprise another functional domain than a nuclease, such as a adenine deaminase domain or a cytosine deaminase domain or a reverse transcriptase domain.

An adenine deaminase (or adenosine deaminase) useful with this invention may be any known or later identified adenine deaminase from any organism (see, e.g., U.S. Pat. No. 10,113,163, which is incorporated by reference herein for its disclosure of adenine deaminases). An adenine deaminase can catalyze the hydrolytic deamination of adenine or adenosine. In some embodiments, the adenine deaminase may catalyze the hydrolytic deamination of adenosine or deoxyadenosine to inosine or deoxyinosine, respectively. In some embodiments, the adenosine deaminase may catalyze the hydrolytic deamination of adenine or adenosine in DNA. In some embodiments, an adenine deaminase encoded by a nucleic acid construct of the invention may generate an A→G conversion in the sense (e.g., “+”; template) strand of the target nucleic acid or a T→C conversion in the antisense (e.g., “−”, complementary) strand of the target nucleic acid.

In some embodiments, an adenosine deaminase may be a variant of a naturally occurring adenine deaminase. Thus, in some embodiments, an adenosine deaminase may be about 70% to 100% identical to a wild type adenine deaminase (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical, and any range or value therein, to a naturally occurring adenine deaminase). In some embodiments, the deaminase or deaminase does not occur in nature and may be referred to as an engineered, mutated or evolved adenosine deaminase. Thus, for example, an engineered, mutated or evolved adenine deaminase polypeptide or an adenine deaminase domain may be about 70% to 99.9% identical to a naturally occurring adenine deaminase polypeptide/domain (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8% or 99.9% identical, and any range or value therein, to a naturally occurring adenine deaminase polypeptide or adenine deaminase domain). In some embodiments, the adenosine deaminase may be from a bacterium, (e.g., Escherichia coli, Staphylococcus aureus, Haemophilus influenzae, Caulobacter crescentus, and the like). In some embodiments, a polynucleotide encoding an adenine deaminase polypeptide/domain may be codon optimized for expression in a plant.

In some embodiments, an adenine deaminase domain may be a wild type tRNA-specific adenosine deaminase domain, e.g., a tRNA-specific adenosine deaminase (TadA) and/or a mutated/evolved adenosine deaminase domain, e.g., mutated/evolved tRNA-specific adenosine deaminase domain (TadA*). In some embodiments, a TadA domain may be from E. coli. In some embodiments, the TadA may be modified, e.g., truncated, missing one or more N-terminal and/or C-terminal amino acids relative to a full-length TadA (e.g., 1, 2, 3, 4, 5, 6, 7, 8, 9, 10, 11, 12, 13, 14, 15, 6, 17, 18, 19, or 20 N-terminal and/or C terminal amino acid residues may be missing relative to a full length TadA. In some embodiments, a TadA polypeptide or TadA domain does not comprise an N-terminal methionine. In some embodiments, a polynucleotide encoding a TadA/TadA* may be codon optimized for expression in a plant.

A cytosine deaminase catalyzes cytosine deamination and results in a thymidine (through an uracil intermediate), causing a C to T conversion, or a G to A conversion in the complementary strand in the genome. Thus, in some embodiments, the cytosine deaminase encoded by the polynucleotide of the invention generates a C→T conversion in the sense (e.g., “+”; template) strand of the target nucleic acid or a G→A conversion in antisense (e.g., “−”, complementary) strand of the target nucleic acid.

In some embodiments, the adenine deaminase encoded by the nucleic acid construct of the invention generates an A→G conversion in the sense (e.g., “+”; template) strand of the target nucleic acid or a T→C conversion in the antisense (e.g., “−”, complementary) strand of the target nucleic acid.

The nucleic acid constructs of the invention encoding a base editor comprising a sequence-specific DNA binding protein and a cytosine deaminase polypeptide, and nucleic acid constructs/expression cassettes/vectors encoding the same, may be used in combination with guide nucleic acids for modifying target nucleic acid including, but not limited to, generation of C-T or G→A mutations in a target nucleic acid including, but not limited to, a plasmid sequence; generation of C→T or G→A mutations in a coding sequence to alter an amino acid identity; generation of C→T or G→A mutations in a coding sequence to generate a stop codon; generation of C→T or G→A mutations in a coding sequence to disrupt a start codon; generation of point mutations in genomic DNA to disrupt transcription factor binding; and/or generation of point mutations in genomic DNA to disrupt splice junctions.

The nucleic acid constructs of the invention encoding a base editor comprising a sequence-specific DNA binding protein and an adenine deaminase polypeptide, and expression cassettes and/or vectors encoding the same may be used in combination with guide nucleic acids for modifying a target nucleic acid including, but not limited to, generation of A→G or T→C mutations in a target nucleic acid including, but not limited to, a plasmid sequence; generation of A→G or T→C mutations in a coding sequence to alter an amino acid identity; generation of A→G or T→C mutations in a coding sequence to generate a stop codon; generation of A→G or T→C mutations in a coding sequence to disrupt a start codon; generation of point mutations in genomic DNA to disrupt function; and/or generation of point mutations in genomic DNA to disrupt splice junctions.

### Guide Nucleic Acids

As used herein, a “guide nucleic acid” refers to a nucleic acid that forms a ribonucleoprotein (e.g., a complex) with a guided nuclease (e.g., without being limiting, Cas12a, CasX) and then guides the ribonucleoprotein to a specific sequence in a target nucleic acid molecule, where the guide nucleic acid and the target nucleic acid molecule share complementary sequences. In an aspect, a ribonucleoprotein provided herein comprises at least one guide nucleic acid.

In an aspect, a guide nucleic acid comprises DNA. In another aspect, a guide nucleic acid comprises RNA. In an aspect, a guide nucleic acid comprises DNA, RNA, or a combination thereof. In an aspect, a guide nucleic acid is single-stranded. In another aspect, a guide nucleic acid is at least partially double-stranded.

When a guide nucleic acid comprises RNA, it can be referred to as a “guide RNA.” In another aspect, a guide nucleic acid comprises DNA and RNA. In another aspect, a guide RNA is single-stranded. In another aspect, a guide RNA is double-stranded. In a further aspect, a guide RNA is partially double-stranded.

A “guide nucleic acid,” “guide RNA,” “gRNA,” “CRISPR RNA/DNA” “crRNA” or “crDNA” as used herein means a nucleic acid that comprises at least one spacer sequence, which is complementary to (and hybridizes to) a target DNA (e.g., protospacer), and at least one repeat sequence (e.g., a repeat of a Type V Cas12a CRISPR-Cas system, or a fragment or portion thereof; a repeat of a Type II Cas9 CRISPR-Cas system, or fragment thereof; a repeat of a Type V C2c1 CRISPR Cas system, or a fragment thereof; a repeat of a CRISPR-Cas system of, for example, C2c3, Cas12a (also referred to as Cpf1), Cas12b, Cas12c, Cas12d, Cas12e, Cas13a, Cas13b, Cas13c, Cas13d, Cas1, Cas1B, Cas2, Cas3, Cas3′, Cas3″, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4 (dinG), and/or Csf5, or a fragment thereof), wherein the repeat sequence may be linked to the 5′ end and/or the 3′ end of the spacer sequence. The design of a gRNA of this invention may be based on a Type I, Type II, Type III, Type IV, Type V, or Type VI CRISPR-Cas system.

In some embodiments, a Cas12a gRNA may comprise, from 5′ to 3′, a repeat sequence (full length or portion thereof (“handle”); e.g., pseudoknot-like structure) and a spacer sequence.

In some embodiments, a guide nucleic acid may comprise more than one repeat sequence-spacer sequence (e.g., 2, 3, 4, 5, 6, 7, 8, 9, 10, or more repeat-spacer sequences) (e.g., repeat-spacer-repeat, e.g., repeat-spacer-repeat-spacer-repeat-spacer-repeat-spacer-repeat-spacer, and the like). The guide nucleic acids of this invention are synthetic, human-made and not found in nature. A gRNA can be quite long and may be used as an aptamer (like in the MS2 recruitment strategy) or other RNA structures hanging off the spacer. A guide RNA may comprise a donor template for introducing specific modifications in the target sequence.

A “repeat sequence” as used herein, refers to, for example, any repeat sequence of a wild-type CRISPR Cas locus (e.g., a Cas9 locus, a Cas12a locus, a C2c1 locus, etc.) or a repeat sequence of a synthetic crRNA that is functional with the CRISPR-Cas effector protein encoded by the nucleic acid constructs of the invention. A repeat sequence useful with this invention can be any known or later identified repeat sequence of a CRISPR-Cas locus (e.g., Type I, Type II, Type III, Type IV, Type V or Type VI) or it can be a synthetic repeat designed to function in a Type I, II, III, IV, V or VI CRISPR-Cas system. A repeat sequence may comprise a hairpin structure and/or a stem loop structure. In some embodiments, a repeat sequence may form a pseudoknot-like structure at its 5′ end (i.e., “handle”). Thus, in some embodiments, a repeat sequence can be identical to or substantially identical to a repeat sequence from wild-type Type I CRISPR-Cas loci, Type II, CRISPR-Cas loci, Type III, CRISPR-Cas loci, Type IV CRISPR-Cas loci, Type V CRISPR-Cas loci and/or Type VI CRISPR-Cas loci. A repeat sequence from a wild-type CRISPR-Cas locus may be determined through established algorithms, such as using the CRISPRfinder offered through CRISPRdb (see, Grissa et al. (2007) Nucleic Acids Res. 35 (Web Server issue): W52-7). In some embodiments, a repeat sequence or portion thereof is linked at its 3′ end to the 5′ end of a spacer sequence, thereby forming a repeat-spacer sequence (e.g., guide nucleic acid, guide RNA/DNA, crRNA, crDNA).

In some embodiments, a repeat sequence comprises, consists essentially of, or consists of at least 10 nucleotides depending on the particular repeat and whether the guide nucleic acid comprising the repeat is processed or unprocessed (e.g., about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 to 100 or more nucleotides, or any range or value therein). In some embodiments, a repeat sequence comprises, consists essentially of, or consists of about 10 to about 20, about 10 to about 30, about 10 to about 45, about 10 to about 50, about 15 to about 30, about 15 to about 40, about 15 to about 45, about 15 to about 50, about 20 to about 30, about 20 to about 40, about 20 to about 50, about 30 to about 40, about 40 to about 80, about 50 to about 100 or more nucleotides.

A repeat sequence linked to the 5′ end of a spacer sequence can comprise a portion of a repeat sequence (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more contiguous nucleotides of a wild type repeat sequence). In some embodiments, a portion of a repeat sequence linked to the 5′ end of a spacer sequence can be about five to about ten consecutive nucleotides in length (e.g., about 5, 6, 7, 8, 9, 10 nucleotides) and have at least 90% sequence identity (e.g., at least about 90%, 91%, 92%, 93%, 94%, 95%, 96%, 97%, 98%, 99%, or more) to the same region (e.g., 5′ end) of a wild type CRISPR Cas repeat nucleotide sequence. In some embodiments, a portion of a repeat sequence may comprise a pseudoknot-like structure at its 5′ end (e.g., “handle”).

A “spacer sequence” as used herein is a nucleotide sequence that is complementary to portion of a target nucleic acid (e.g., target DNA) (e.g., protospacer). A spacer sequence can be fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target nucleic acid. In some embodiments, the spacer sequence can have one, two, three, four, or five mismatches as compared to the target nucleic acid, which mismatches can be contiguous or noncontiguous. In some embodiments, the spacer sequence can have 70% complementarity to a target nucleic acid. In other embodiments, the spacer nucleotide sequence can have 80% complementarity to a target nucleic acid. In still other embodiments, the spacer nucleotide sequence can have 85%, 90%, 95%, 96%, 97%, 98%, 99% or 99.5% complementarity, and the like, to the target nucleic acid (protospacer). In some embodiments, the spacer sequence is 100% complementary to the target nucleic acid. A spacer sequence may have a length from about 15 nucleotides to about 30 nucleotides (e.g., 15, 16, 17, 18, 19, 20, 21, 22, 23, 24, 25, 26, 27, 28, 29, or 30 nucleotides, or any range or value therein). Thus, in some embodiments, a spacer sequence may have complete complementarity or substantial complementarity over a region of a target nucleic acid (e.g., protospacer) that is at least about 15 nucleotides to about 30 nucleotides in length. In some embodiments, the spacer is about 20 nucleotides in length. In some embodiments, the spacer is about 21, 22, or 23 nucleotides in length. In some embodiments, a spacer sequence may comprise any one of the sequences of SEQ ID NOs:88-90, or any combination thereof.

In some embodiments, the 5′ region of a spacer sequence of a guide nucleic acid may be identical to a target DNA, while the 3′ region of the spacer may be substantially complementary to the target DNA (such as a spacer of a Type V CRISPR-Cas system), or the 3′ region of a spacer sequence of a guide nucleic acid may be identical to a target DNA, while the 5′ region of the spacer may be substantially complementary to the target DNA (such as a spacer of a Type II CRISPR-Cas system), and therefore, the overall complementarity of the spacer sequence to the target DNA may be less than 100%. Thus, for example, in a guide for a Type V CRISPR-Cas system, the first 1, 2, 3, 4, 5, 6, 7, 8, 9, 10 nucleotides in the 5′ region (i.e., seed region) of, for example, a 20 nucleotide spacer sequence may be 100% complementary to the target DNA, while the remaining nucleotides in the 3′ region of the spacer sequence are substantially complementary (e.g., at least about 70% complementary) to the target DNA. In some embodiments, the first 1 to 8 nucleotides (e.g., the first 1, 2, 3, 4, 5, 6, 7, 8, nucleotides, and any range therein) of the 5′ end of the spacer sequence may be 100% complementary to the target DNA, while the remaining nucleotides in the 3′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA.

As a further example, in a guide for a Type II CRISPR-Cas system, the first 1, 2, 3, 4, 5, 6, 7, 8, 9, 10 nucleotides in the 3′ region (i.e., seed region) of, for example, a 20 nucleotide spacer sequence may be 100% complementary to the target DNA, while the remaining nucleotides in the 5′ region of the spacer sequence are substantially complementary (e.g., at least about 70% complementary) to the target DNA. In some embodiments, the first 1 to 10 nucleotides (e.g., the first 1, 2, 3, 4, 5, 6, 7, 8, 9, 10 nucleotides, and any range therein) of the 3′ end of the spacer sequence may be 100% complementary to the target DNA, while the remaining nucleotides in the 5′ region of the spacer sequence are substantially complementary (e.g., at least about 50% complementary (e.g., at least about 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more or any range or value therein)) to the target DNA.

In some embodiments, a seed region of a spacer may be about 8 to about 10 nucleotides in length, about 5 to about 6 nucleotides in length, or about 6 nucleotides in length.

In an aspect, a guide nucleic acid comprises a guide RNA. In another aspect, a guide nucleic acid comprises at least one guide RNA. In another aspect, a guide nucleic acid comprises at least two guide RNAs. In another aspect, a guide nucleic acid comprises at least three guide RNAs. In another aspect, a guide nucleic acid comprises at least five guide RNAs. In another aspect, a guide nucleic acid comprises at least ten guide RNAs.

In another aspect, a guide nucleic acid comprises at least 10 nucleotides. In another aspect, a guide nucleic acid comprises at least 11 nucleotides. In another aspect, a guide nucleic acid comprises at least 12 nucleotides. In another aspect, a guide nucleic acid comprises at least 13 nucleotides. In another aspect, a guide nucleic acid comprises at least 14 nucleotides. In another aspect, a guide nucleic acid comprises at least 15 nucleotides. In another aspect, a guide nucleic acid comprises at least 16 nucleotides. In another aspect, a guide nucleic acid comprises at least 17 nucleotides. In another aspect, a guide nucleic acid comprises at least 18 nucleotides. In another aspect, a guide nucleic acid comprises at least 19 nucleotides. In another aspect, a guide nucleic acid comprises at least 20 nucleotides. In another aspect, a guide nucleic acid comprises at least 21 nucleotides. In another aspect, a guide nucleic acid comprises at least 22 nucleotides. In another aspect, a guide nucleic acid comprises at least 23 nucleotides. In another aspect, a guide nucleic acid comprises at least 24 nucleotides. In another aspect, a guide nucleic acid comprises at least 25 nucleotides. In another aspect, a guide nucleic acid comprises at least 26 nucleotides. In another aspect, a guide nucleic acid comprises at least 27 nucleotides. In another aspect, a guide nucleic acid comprises at least 28 nucleotides. In another aspect, a guide nucleic acid comprises at least 30 nucleotides. In another aspect, a guide nucleic acid comprises at least 35 nucleotides. In another aspect, a guide nucleic acid comprises at least 40 nucleotides. In another aspect, a guide nucleic acid comprises at least 45 nucleotides. In another aspect, a guide nucleic acid comprises at least 50 nucleotides.

In another aspect, a guide nucleic acid comprises between 10 nucleotides and 50 nucleotides. In another aspect, a guide nucleic acid comprises between 10 nucleotides and 40 nucleotides. In another aspect, a guide nucleic acid comprises between 10 nucleotides and 30 nucleotides. In another aspect, a guide nucleic acid comprises between 10 nucleotides and 20 nucleotides. In another aspect, a guide nucleic acid comprises between 16 nucleotides and 28 nucleotides. In another aspect, a guide nucleic acid comprises between 16 nucleotides and 25 nucleotides. In another aspect, a guide nucleic acid comprises between 16 nucleotides and 20 nucleotides.

In an aspect, a guide nucleic acid comprises at least 70% sequence complementarity to a target site. In an aspect, a guide nucleic acid comprises at least 75% sequence complementarity to a target site. In an aspect, a guide nucleic acid comprises at least 80% sequence complementarity to a target site. In an aspect, a guide nucleic acid comprises at least 85% sequence complementarity to a target site. In an aspect, a guide nucleic acid comprises at least 90% sequence complementarity to a target site. In an aspect, a guide nucleic acid comprises at least 91% sequence complementarity to a target site. In an aspect, a guide nucleic acid comprises at least 92% sequence complementarity to a target site. In an aspect, a guide nucleic acid comprises at least 93% sequence complementarity to a target site. In an aspect, a guide nucleic acid comprises at least 94% sequence complementarity to a target site. In an aspect, a guide nucleic acid comprises at least 95% sequence complementarity to a target site. In an aspect, a guide nucleic acid comprises at least 96% sequence complementarity to a target site. In an aspect, a guide nucleic acid comprises at least 97% sequence complementarity to a target site. In an aspect, a guide nucleic acid comprises at least 98% sequence complementarity to a target site. In an aspect, a guide nucleic acid comprises at least 99% sequence complementarity to a target site. In an aspect, a guide nucleic acid comprises 100% sequence complementarity to a target site. In another aspect, a guide nucleic acid comprises between 70% and 100% sequence complementarity to a target site. In another aspect, a guide nucleic acid comprises between 80% and 100% sequence complementarity to a target site. In another aspect, a guide nucleic acid comprises between 90% and 100% sequence complementarity to a target site.

In an aspect, a guide nucleic acid is capable of hybridizing to a target site.

As noted above, some guided nucleases, such as CasX and Cas9, require another non-coding RNA component, referred to as a trans-activating crRNA (tracrRNA), to have functional activity. Guide nucleic acid molecules provided herein can combine a crRNA and a tracrRNA into one nucleic acid molecule in what is herein referred to as a “single guide RNA” (sgRNA). The gRNA guides the active CasX complex to a target site within a target sequence, where CasX can cleave the target site. In other embodiments, the crRNA and tracrRNA are provided as separate nucleic acid molecules.

In an aspect, a guide nucleic acid comprises a crRNA. In another aspect, a guide nucleic acid comprises a tracrRNA. In a further aspect, a guide nucleic acid comprises a sgRNA.

### Target Sites

As used herein, a “target sequence” refers to a selected sequence or region of a DNA molecule in which a modification (e.g., cleavage, site-directed integration) is desired. A target sequence comprises a target site.

As used herein, a “target site” refers to the portion of a target sequence that is cleaved by a guided nuclease such as CRISPR nuclease. In contrast to a non-target nucleic acid (e.g., non-target ssDNA) or non-target region, a target site comprises significant complementarity to a guide nucleic acid or a guide RNA.

In an aspect, a target site is 100% complementary to a guide nucleic acid. In another aspect, a target site is 99% complementary to a guide nucleic acid. In another aspect, a target site is 98% complementary to a guide nucleic acid. In another aspect, a target site is 97% complementary to a guide nucleic acid. In another aspect, a target site is 96% complementary to a guide nucleic acid. In another aspect, a target site is 95% complementary to a guide nucleic acid. In another aspect, a target site is 94% complementary to a guide nucleic acid. In another aspect, a target site is 93% complementary to a guide nucleic acid. In another aspect, a target site is 92% complementary to a guide nucleic acid. In another aspect, a target site is 91% complementary to a guide nucleic acid. In another aspect, a target site is 90% complementary to a guide nucleic acid. In another aspect, a target site is 85% complementary to a guide nucleic acid. In another aspect, a target site is 80% complementary to a guide nucleic acid.

In an aspect, a target site comprises at least one PAM site. In an aspect, a target site is adjacent to a nucleic acid sequence that comprises at least one PAM site. In another aspect, a target site is within 5 nucleotides of at least one PAM site. In a further aspect, a target site is within 10 nucleotides of at least one PAM site. In another aspect, a target site is within 15 nucleotides of at least one PAM site. In another aspect, a target site is within 20 nucleotides of at least one PAM site. In another aspect, a target site is within 25 nucleotides of at least one PAM site. In another aspect, a target site is within 30 nucleotides of at least one PAM site.

In an aspect, a target site is positioned within genic DNA. In another aspect, a target site is positioned within a gene. In another aspect, a target site is positioned within a gene of interest. In another aspect, a target site is positioned within an exon of a gene. In another aspect, a target site is positioned within an intron of a gene. In another aspect, a target site is positioned within the promoter of a gene. In another aspect, a target site is positioned within 5′-UTR of a gene. In another aspect, a target site is positioned within a 3′-UTR of a gene. In another aspect, a target site is positioned within intergenic DNA.

A “protospacer sequence” refers to the target double stranded DNA and specifically to the portion of the target DNA (e.g., or target region in the genome) that is fully or substantially complementary (and hybridizes) to the spacer sequence of the CRISPR repeat-spacer sequences (e.g., guide nucleic acids, CRISPR arrays, crRNAs).

In the case of Type V CRISPR-Cas (e.g., Cas12a) systems and Type II CRISPR-Cas (Cas9) systems, the protospacer sequence is flanked by (e.g., immediately adjacent to) a protospacer adjacent motif (PAM). For Type IV CRISPR-Cas systems, the PAM is located at the 5′ end on the non-target strand and at the 3′ end of the target strand (see below, as an example).

```
    5′-NNNNNNNNNNNNNNNNNNN-3′ RNA Spacer
          |||||||||||||||||||
3′AAANNNNNNNNNNNNNNNNNNN-5′ Target strand
    |||
5′TTTNNNNNNNNNNNNNNNNNNN-3′ Non-target strand
```

In the case of Type II CRISPR-Cas (e.g., Cas9) systems, the PAM is located immediately 3′ of the target region. The PAM for Type I CRISPR-Cas systems is located 5′ of the target strand. There is no known PAM for Type III CRISPR-Cas systems. Makarova et al. describes the nomenclature for all the classes, types and subtypes of CRISPR systems ((2015) Nature Reviews Microbiology 13:722-736). Guide structures and PAMs are described in by R. Barrangou ((2015) Genome Biol. 16:247).

Canonical Cas12a PAMs are T rich. In some embodiments, a canonical Cas12a PAM sequence may be 5′-TTN, 5′-TTTN, or 5′-TTTV. In some embodiments, canonical Cas9 (e.g., S. pyogenes) PAMs may be 5′-NGG-3′. In some embodiments, non-canonical PAMs may be used but may be less efficient.

Additional PAM sequences may be determined by those skilled in the art through established experimental and computational approaches. Thus, for example, experimental approaches include targeting a sequence flanked by all possible nucleotide sequences and identifying sequence members that do not undergo targeting, such as through the transformation of target plasmid DNA (Esvelt et al. (2013) Nat. Methods 10:1116-1121; Jiang et al. (2013) Nat. Biotechnol. 31:233-239). In some aspects, a computational approach can include performing BLAST searches of natural spacers to identify the original target DNA sequences in bacteriophages or plasmids and aligning these sequences to determine conserved sequences adjacent to the target sequence (Briner and Barrangou. (2014) Appl. Environ. Microbiol. 80:994-1001; Mojica et al. (2009) Microbiology 155:733-740).

In an aspect, a target DNA molecule is single-stranded. In another aspect, a target DNA molecule is double-stranded.

In an aspect, a target sequence comprises genomic DNA. In an aspect, a target sequence is positioned within a nuclear genome. In an aspect, a target sequence comprises chromosomal DNA. In an aspect, a target sequence comprises plasmid DNA. In an aspect, a target sequence is positioned within a plasmid. In an aspect, a target sequence comprises mitochondrial DNA. In an aspect, a target sequence is positioned within a mitochondrial genome. In an aspect, a target sequence comprises plastid DNA. In an aspect, a target sequence is positioned within a plastid genome. In an aspect, a target sequence comprises chloroplast DNA. In an aspect, a target sequence is positioned within a chloroplast genome. In an aspect, a target sequence is positioned within a genome selected from the group consisting of a nuclear genome, a mitochondrial genome, and a plastid genome.

In an aspect, a target sequence comprises genic DNA. As used herein, “genic DNA” refers to DNA that encodes one or more genes. In another aspect, a target sequence comprises intergenic DNA. In contrast to genic DNA, “intergenic DNA” comprises noncoding DNA, and lacks DNA encoding a gene. In an aspect, intergenic DNA is positioned between two genes.

In an aspect, a target sequence encodes a gene. As used herein, a “gene” refers to a polynucleotide that can produce a functional unit (e.g., without being limiting, for example, a protein, or a non-coding RNA molecule). A gene can comprise a promoter, an enhancer sequence, a leader sequence, a transcriptional start site, a transcriptional stop site, a polyadenylation site, one or more exons, one or more introns, a 5′-UTR, a 3′-UTR, or any combination thereof. A “gene sequence” can comprise a polynucleotide sequence encoding a promoter, an enhancer sequence, a leader sequence, a transcriptional start site, a transcriptional stop site, a polyadenylation site, one or more exons, one or more introns, a 5′-UTR, a 3′-UTR, or any combination thereof. In one aspect, a gene encodes a non-protein-coding RNA molecule or a precursor thereof. In another aspect, a gene encodes a protein. In some embodiments, the target sequence is selected from the group consisting of: a promoter, an enhancer sequence, a leader sequence, a transcriptional start site, a transcriptional stop site, a polyadenylation site, an exon, an intron, a splice site, a 5′-UTR, a 3′-UTR, a protein coding sequence, a non-protein-coding sequence, a miRNA, a pre-miRNA and a miRNA binding site.

Non-limiting examples of a non-protein-coding RNA molecule include a microRNA (miRNA), a miRNA precursor (pre-miRNA), a small interfering RNA (siRNA), a small RNA (18 to 26 nucleotides in length) and precursor encoding same, a heterochromatic siRNA (hc-siRNA), a Piwi-interacting RNA (piRNA), a hairpin double strand RNA (hairpin dsRNA), a trans-acting siRNA (ta-siRNA), a naturally occurring antisense siRNA (nat-siRNA), a CRISPR RNA (crRNA), a tracer RNA (tracrRNA), a guide RNA (gRNA), and a single guide RNA (sgRNA). In an aspect, a non-protein-coding RNA molecule comprises a miRNA. In an aspect, a non-protein-coding RNA molecule comprises a siRNA. In an aspect, a non-protein-coding RNA molecule comprises a ta-siRNA. In an aspect, a non-protein-coding RNA molecule is selected from the group consisting of a miRNA, a siRNA, and a ta-siRNA.

As used herein, a “gene of interest” refers to a polynucleotide sequence encoding a protein or a non-protein-coding RNA molecule that is to be integrated into a target sequence, or, alternatively, an endogenous polynucleotide sequence encoding a protein or a non-protein-coding RNA molecule that is to be edited by a ribonucleoprotein. In an aspect, a gene of interest encodes a protein. In another aspect, a gene of interest encodes a non-protein-coding RNA molecule. In an aspect, a gene of interest is exogenous to a targeted DNA molecule. In an aspect, a gene of interest replaces an endogenous gene in a targeted DNA molecule.

### Mutations

As used herein, the term mutation or edit is used interchangeably.

In an aspect, a ribonucleoprotein or method provided herein generates at least one mutation or edit in a target sequence.

In an aspect, a seed produced from a plant provided herein comprises at least one mutation or edit in a gene of interest comprising a target site as compared to a seed of a control plant of the same line or variety that lacks a first nucleic acid sequence encoding a guided nuclease. In an aspect, a seed produced from a plant provided herein comprises at least one mutation or edit in a gene of interest comprising a target site as compared to a seed of a control plant of the same line or variety that lacks a first nucleic acid sequence encoding a guided.

In an aspect, a seed produced from a plant provided herein comprises at least one mutation or edit in a gene of interest comprising a target site as compared to a seed of a control plant of the same line or variety that lacks a first nucleic acid sequence encoding a guided nuclease operably linked to a heterologous promoter and/or a second nucleic acid encoding at least one guide nucleic acid operably linked to a floral cell-preferred promoter. In an aspect, a seed produced from a plant provided herein comprises at least one mutation or edit in a gene of interest comprising a target site as compared to a seed of a control plant of the same line or variety that lacks a first nucleic acid sequence encoding a guided nuclease operably linked to a heterologous promoter or a second nucleic acid encoding at least one guide nucleic acid.

As used herein, a “mutation” or “edit” refers to a non-naturally occurring alteration to a nucleic acid or amino acid sequence as compared to a naturally occurring reference nucleic acid or amino acid sequence from the same organism. It will be appreciated that, when identifying a mutation or edit, the reference sequence should be from the same nucleic acid (e.g, gene, non-coding RNA) or amino acid (e.g, protein). In determining if a difference between two sequences comprises a mutation or edit, it will be appreciated in the art that the comparison should not be made between homologous sequences of two different species or between homologous sequences of two different varieties of a single species. Rather, the comparison should be made between the edited (e.g., mutated) sequence and the endogenous, non-edited (e.g., “wildtype”) sequence of the same organism.

Several types of mutations are known in the art. In an aspect, a mutation or edit comprises an insertion. An “insertion” refers to the addition of one or more nucleotides or amino acids to a given polynucleotide or amino acid sequence, respectively, as compared to an endogenous reference polynucleotide or amino acid sequence. In another aspect, a mutation or edit comprises a deletion. A “deletion” refers to the removal of one or more nucleotides or amino acids to a given polynucleotide or amino acid sequence, respectively, as compared to an endogenous reference polynucleotide or amino acid sequence. In another aspect, a mutation or edit comprises a substitution. A “substitution” refers to the replacement of one or more nucleotides or amino acids to a given polynucleotide or amino acid sequence, respectively, as compared to an endogenous reference polynucleotide or amino acid sequence. In another aspect, a mutation or edit comprises an inversion. An “inversion” refers to when a segment of a polynucleotide or amino acid sequence is reversed end-to-end. In an aspect, a mutation provided herein comprises a mutation selected from the group consisting of an insertion, a deletion, a substitution, and an inversion.

In an aspect, a plant or seed comprises at least one mutation or edit in a gene of interest, where the at least one mutation or edit results in the deletion of one or more amino acids from a protein encoded by the gene of interest as compared to a wildtype protein.

In an aspect, a plant or seed comprises at least one mutation or edit in a gene of interest, where the at least one mutation or edit results in the substitution of one or more amino acids within a protein encoded by the gene of interest as compared to a wildtype protein.

In an aspect, a plant or seed comprises at least one mutation or edit in a gene of interest, where the at least one mutation or edit results in the insertion of one or more amino acids within a protein encoded by the gene of interest as compared to a wildtype protein.

Mutations/Edits in coding regions of genes (e.g., exonic mutations) can result in a truncated protein or polypeptide when a mutated messenger RNA (mRNA) is translated into a protein or polypeptide. In an aspect, this disclosure provides a mutation or edit that results in the truncation of a protein or polypeptide. As used herein, a “truncated” protein or polypeptide comprises at least one fewer amino acid as compared to an endogenous control protein or polypeptide. For example, if endogenous Protein A comprises 100 amino acids, a truncated version of Protein A can comprise between 1 and 99 amino acids.

Without being limited by any scientific theory, one way to cause a protein or polypeptide truncation is by the introduction of a premature stop codon in an mRNA transcript of an endogenous gene. In an aspect, this disclosure provides a mutation that results in a premature stop codon in an mRNA transcript of an endogenous gene. As used herein, a “stop codon” refers to a nucleotide triplet within an mRNA transcript that signals a termination of protein translation. A “premature stop codon” refers to a stop codon positioned earlier (e.g., on the 5′-side) than the normal stop codon position in an endogenous mRNA transcript. Without being limiting, several stop codons are known in the art, including “UAG,” “UAA,” “UGA,” “TAG,” “TAA,” and “TGA.”

In an aspect, a seed or plant comprises at least one mutation or edit, where the at least one mutation or edit results in the introduction of a premature stop codon in a messenger RNA encoded by the gene of interest as compared to a wildtype messenger RNA.

In an aspect, a mutation or edit provided herein comprises a null mutation. As used herein, a “null mutation” refers to a mutation that confers a complete loss-of-function for a protein encoded by a gene comprising the mutation, or, alternatively, a mutation that confers a complete loss-of-function for a small RNA encoded by a genomic locus. A null mutation can cause lack of mRNA transcript production, a lack of small RNA transcript production, a lack of protein function, or a combination thereof.

A mutation or edit provided herein can be positioned in any part of an endogenous gene. In an aspect, a mutation or edit provided herein is positioned within an exon of an endogenous gene. In another aspect, a mutation or edit provided herein is positioned within an intron of an endogenous gene. In a further aspect, a mutation or edit provided herein is positioned within a 5′-untranslated region of an endogenous gene. In still another aspect, a mutation or edit provided herein is positioned within a 3′-untranslated region of an endogenous gene. In yet another aspect, a mutation or edit provided herein is positioned within a promoter of an endogenous gene.

In an aspect, a mutation or edit is positioned at a splice site within a gene. A mutation at a splice site can interfere with the splicing of exons during mRNA processing. If one or more nucleotides are inserted, deleted, or substituted at a splice site, splicing can be perturbed. Perturbed splicing can result in unspliced introns, missing exons, or both, from a mature mRNA sequence. Typically, although not always, a “GU” sequence is required at the 5′ end of an intron and a “AG” sequence is required at the 3′ end of an intron for proper splicing. If either of these splice sites are mutated, splicing perturbations can occur.

In an aspect, a seed or plant comprises at least one mutation or edit, where the at least one mutation or edit comprises the deletion of one or more splice sites from a gene of interest. In another aspect, a seed or plant comprises at least one mutation or edit, where the at least one mutation or edit is positioned within one or more splice sites from a gene of interest.

In an aspect, a mutation or edit comprises a site-directed integration. In an aspect, a site-directed integration comprises the insertion of all or part of a desired sequence into a target sequence.

As used herein, “site-directed integration” refers to all, or a portion, of a desired sequence (e.g., an exogenous gene, an edited endogenous gene) being inserted or integrated at a desired site or locus within the plant genome (e.g., target sequence). As used herein, a “desired sequence” refers to a DNA molecule comprising a nucleic acid sequence that is to be integrated into a genome of a plant or plant cell. The desired sequence can comprise a transgene or construct. In an aspect, a nucleic acid molecule comprising a desired sequence comprises one or two homology arms flanking the desired sequence to promote the targeted insertion event through homologous recombination and/or homology-directed repair.

In an aspect, a method provided herein comprises site-directed integration of a desired sequence into a target sequence.

Any site or locus within the genome of a plant can be chosen for site-directed integration of a transgene or construct of the present disclosure. In an aspect, a target sequence is positioned within a B, or supernumerary, chromosome.

In an aspect, a method provided herein further comprises detecting an edit or a mutation in a target sequence. The screening and selection of mutagenized or edited plants or plant cells can be through any methodologies known to those having ordinary skill in the art. Examples of screening and selection methodologies include, but are not limited to, Southern analysis, PCR amplification for detection of a polynucleotide, Northern blots, RNase protection, primer-extension, RT-PCR amplification for detecting RNA transcripts, Sanger sequencing, Next Generation sequencing technologies (e.g., Illumina, PacBio, Ion Torrent, 454) enzymatic assays for detecting enzyme or ribozyme activity of polypeptides and polynucleotides, protein gel electrophoresis, Western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides. Methods for performing all of the above-referenced techniques are known in the art.

In an aspect, a sequence provided herein encodes at least one ribozyme. In an aspect, a sequence provided herein encodes at least two ribozymes. In an aspect, a ribozyme is a self-cleaving ribozyme. Self-cleaving ribozymes are known in the art. For example, see Jimenez et al., Trends Biochem. Sci., 40:648-661 (2015).

In an aspect, a sequence encoding at least one guide nucleic acid is flanked by self-cleaving ribozymes. In an aspect, a sequence encoding at least one guide nucleic acid is immediately adjacent to a sequence encoding a ribozyme (e.g., the 5′-most nucleotide of the guide nucleic acid abuts the 3′-most nucleotide of the ribozyme or the 3′-most nucleotide of the guide nucleic acid abuts the 5′-most nucleotide of the ribozyme). In an aspect, a sequence encoding at least one guide nucleic acid is separated from a sequence encoding a ribozyme by at least 1, at least 2, at least 3, at least 4, at least 5, at least 10, at least 20, at least 30, at least 40, at least 50, at least 100, at least 250, at least 500, or at least 10000 nucleotides.

### Plants

Any plant or plant cell can be used with the methods and compositions provided herein. In an aspect, a plant is selected from the group consisting of a corn plant, a rice plant, a sorghum plant, a wheat plant, an alfalfa plant, a barley plant, a millet plant, a rye plant, a sugarcane plant, a cotton plant, a soybean plant, a canola plant, a tomato plant, an onion plant, a cucumber plant, an Arabidopsis plant, and a potato plant. In an aspect, a plant is an angiosperm. In an aspect, a plant is a gymnosperm. In an aspect, a plant is a monocotyledonous plant. In an aspect, a plant is a dicotyledonous plant. In an aspect, a plant is a plant of a family selected from the group consisting of Alliaceae, Anacardiaceae, Apiaceae, Arecaceae, Asteraceae, Brassicaceae, Caesalpiniaceae, Cucurbitaceae, Ericaceae, Fabaceae, Juglandaceae, Malvaceae, Mimosaceae, Moraceae, Musaceae, Orchidaceae, Papilionaceae, Pinaceae, Poaceae, Rosaceae, Rutaceae, Rubiaceae, and Solanaceae.

In an aspect, a plant cell is selected from the group consisting of a corn cell, a rice cell, a sorghum cell, a wheat cell, an alfalfa cell, a barley cell, a millet cell, a rye cell, a sugarcane cell, a cotton cell, a soybean cell, a canola cell, a tomato cell, an onion cell, a cucumber cell, an Arabidopsis cell, and a potato cell. In an aspect, a plant cell is an angiosperm plant cell. In an aspect, a plant cell is a gymnosperm plant cell. In an aspect, a plant cell is a monocotyledonous plant cell. In an aspect, a plant cell is a dicotyledonous plant cell. In an aspect, a plant cell is a plant cell of a family selected from the group consisting of Alliaceae, Anacardiaceae, Apiaceae, Arecaceae, Asteraceae, Brassicaceae, Caesalpiniaceae, Cucurbitaceae, Ericaceae, Fabaceae, Juglandaceae, Malvaceae, Mimosaceae, Moraceae, Musaceae, Orchidaceae, Papilionaceae, Pinaceae, Poaceae, Rosaceae, Rutaceae, Rubiaceae, and Solanaceae.

As used herein, a “variety” refers to a group of plants within a species (e.g., without being limiting Zea mays) that share certain genetic traits that separate them from other possible varieties within that species. Varieties can be inbreds or hybrids, though commercial plants are often hybrids to take advantage of hybrid vigor. Individuals within a hybrid cultivar are homogeneous, nearly genetically identical, with most loci in the heterozygous state.

As used herein, the term “inbred” means a line that has been bred for genetic homogeneity. In an aspect, a seed provided herein is an inbred seed. In an aspect, a plant provided herein is an inbred plant.

As used herein, the term “hybrid” means a progeny of mating between at least two genetically dissimilar parents. Without limitation, examples of mating schemes include single crosses, modified single cross, double modified single cross, three-way cross, modified three-way cross, and double cross wherein at least one parent in a modified cross is the progeny of a cross between sister lines. In an aspect, a seed provided herein is a hybrid seed. In an aspect, a plant provided herein is a hybrid plant.

In some jurisdictions, products obtained exclusively by essentially biological processes, such as plant products are excluded from patent protection. Accordingly, the claimed plants, plant parts and cells and their progeny can be defined as directed only to those plants, plant parts and cells and their progeny which are obtained by technical intervention (regardless of any further propagation through crossing and selection). An embodiment of the invention is directed at plants, or plant parts or progeny produced or obtainable using gene editing technology herein described. Alternatively, the subject matter excluded from patentability may be disclaimed. An embodiment of the invention is directed at plants, part of plants or progeny thereof comprising the genomic alterations as elsewhere herein described, provided that the plants, parts or plants or progeny are not obtained exclusively through essentially biological processes, wherein essentially biological processes are processes for the production of plants or animals if they consist entirely of natural phenomena such as crossing or selection.

### Transformation

Methods can involve transient transformation or stable integration of any nucleic acid molecule into any plant or plant cell provided herein.

As used herein, “stable integration” or “stably integrated” refers to a transfer of DNA into genomic DNA of a targeted cell or plant that allows the targeted cell or plant to pass the transferred DNA to the next generation of the transformed organism. Stable transformation requires the integration of transferred DNA within the reproductive cell(s) of the transformed organism. As used herein, “transiently transformed” or “transient transformation” refers to a transfer of DNA into a cell that is not transferred to the next generation of the transformed organism. In a transient transformation the transformed DNA does not typically integrate into the transformed cell's genomic DNA. In one aspect, a method stably transforms a plant cell or plant with one or more nucleic acid molecules provided herein. In another aspect, a method transiently transforms a plant cell or plant with one or more nucleic acid molecules provided herein.

In an aspect, a nucleic acid molecule encoding a guided nuclease is stably integrated into a genome of a plant. In an aspect, a nucleic acid molecule encoding a Cas12a nuclease is stably integrated into a genome of a plant. In an aspect, a nucleic acid molecule encoding a CasX nuclease is stably integrated into a genome of a plant. In an aspect, a nucleic acid molecule encoding a guide nucleic acid is stably integrated into a genome of a plant. In an aspect, a nucleic acid molecule encoding a guide RNA is stably integrated into a genome of a plant. In an aspect, a nucleic acid molecule encoding a single-guide RNA is stably integrated into a genome of a plant.

Numerous methods for transforming cells with a recombinant nucleic acid molecule or construct are known in the art, which can be used according to methods of the present application. Any suitable method or technique for transformation of a cell known in the art can be used according to present methods. Effective methods for transformation of plants include bacterially mediated transformation, such as Agrobacterium-mediated or Rhizobium-mediated transformation and microprojectile bombardment-mediated transformation. A variety of methods are known in the art for transforming explants with a transformation vector via bacterially mediated transformation or microprojectile bombardment and then subsequently culturing, etc., those explants to regenerate or develop transgenic plants.

In an aspect, a method comprises providing a cell with a nucleic acid molecule via Agrobacterium-mediated transformation. In an aspect, a method comprises providing a cell with a nucleic acid molecule via polyethylene glycol-mediated transformation. In an aspect, a method comprises providing a cell with a nucleic acid molecule via biolistic transformation. In an aspect, a method comprises providing a cell with a nucleic acid molecule via liposome-mediated transfection. In an aspect, a method comprises providing a cell with a nucleic acid molecule via viral transduction. In an aspect, a method comprises providing a cell with a nucleic acid molecule via use of one or more delivery particles. In an aspect, a method comprises providing a cell with a nucleic acid molecule via microinjection. In an aspect, a method comprises providing a cell with a nucleic acid molecule via electroporation.

In an aspect, a nucleic acid molecule is provided to a cell via a method selected from the group consisting of Agrobacterium-mediated transformation, polyethylene glycol-mediated transformation, biolistic transformation, liposome-mediated transfection, viral transduction, the use of one or more delivery particles, microinjection, and electroporation.

Other methods for transformation, such as vacuum infiltration, pressure, sonication, and silicon carbide fiber agitation, are also known in the art and envisioned for use with any method provided herein.

Methods of transforming cells are well known by persons of ordinary skill in the art. For instance, specific instructions for transforming plant cells by microprojectile bombardment with particles coated with recombinant DNA (e.g., biolistic transformation) are found in U.S. Pat. Nos. 5,550,318; 5,538,880 6,160,208; 6,399,861; and 6,153,812 and Agrobacterium-mediated transformation is described in U.S. Pat. Nos. 5,159,135; 5,824,877; 5,591,616; 6,384,301; 5,750,871; 5,463,174; and 5,188,958, all of which are incorporated herein by reference. Additional methods for transforming plants can be found in, for example, Compendium of Transgenic Crop Plants (2009) Blackwell Publishing. Any appropriate method known to those skilled in the art can be used to transform a plant cell with any of the nucleic acid molecules provided herein.

Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

Delivery vehicles, vectors, particles, nanoparticles, formulations and components thereof for expression of one or more elements of a nucleic acid molecule are as used in WO 2014/093622. In an aspect, a method of providing a nucleic acid molecule or a protein to a cell comprises delivery via a delivery particle. In an aspect, a method of providing a nucleic acid molecule to a plant cell or plant comprises delivery via a delivery vesicle. In an aspect, a delivery vesicle is selected from the group consisting of an exosome and a liposome. In an aspect, a method of providing a nucleic acid molecule to a plant cell or plant comprises delivery via a viral vector. In an aspect, a viral vector is selected from the group consisting of an adenovirus vector, a lentivirus vector, and an adeno-associated viral vector. In another aspect, a method providing a nucleic acid molecule to a plant cell or plant comprises delivery via a nanoparticle. In an aspect, a method providing a nucleic acid molecule to a plant cell or plant comprises microinjection. In an aspect, a method providing a nucleic acid molecule to a plant cell or plant comprises polycations. In an aspect, a method providing a nucleic acid molecule to a plant cell or plant comprises a cationic oligopeptide.

In an aspect, a delivery particle is selected from the group consisting of an exosome, an adenovirus vector, a lentivirus vector, an adeno-associated viral vector, a nanoparticle, a polycation, and a cationic oligopeptide. In an aspect, a method provided herein comprises the use of one or more delivery particles. In another aspect, a method provided herein comprises the use of two or more delivery particles. In another aspect, a method provided herein comprises the use of three or more delivery particles.

Suitable agents to facilitate transfer of nucleic acids into a plant cell include agents that increase permeability of the exterior of the plant or that increase permeability of plant cells to oligonucleotides or polynucleotides. Such agents to facilitate transfer of the composition into a plant cell include a chemical agent, or a physical agent, or combinations thereof. Chemical agents for conditioning includes (a) surfactants, (b) organic solvents, aqueous solutions, or aqueous mixtures of organic solvents, (c) oxidizing agents, (e) acids, (f) bases, (g) oils, (h) enzymes, or combinations thereof.

Organic solvents useful in conditioning a plant to permeation by polynucleotides include DMSO, DMF, pyridine, N-pyrrolidine, hexamethylphosphoramide, acetonitrile, dioxane, polypropylene glycol, other solvents miscible with water or that will dissolve phosphonucleotides in non-aqueous systems (such as is used in synthetic reactions). Naturally derived or synthetic oils with or without surfactants or emulsifiers can be used, e. g., plant-sourced oils, crop oils (such as those listed in the 9^{th} Compendium of Herbicide Adjuvants, publicly available on line at www(dot)herbicide(dot)adjuvants(dot)com) can be used, e. g., paraffinic oils, polyol fatty acid esters, or oils with short-chain molecules modified with amides or polyamines such as polyethyleneimine or N-pyrrolidine.

Examples of useful surfactants include sodium or lithium salts of fatty acids (such as tallow or tallowamines or phospholipids) and organosilicone surfactants. Other useful surfactants include organosilicone surfactants including nonionic organosilicone surfactants, e. g., trisiloxane ethoxylate surfactants or a silicone polyether copolymer such as a copolymer of polyalkylene oxide modified heptamethyl trisiloxane and allyloxypolypropylene glycol methylether (commercially available as Silwet® L-77).

Useful physical agents can include (a) abrasives such as carborundum, corundum, sand, calcite, pumice, garnet, and the like, (b) nanoparticles such as carbon nanotubes or (c) a physical force. Carbon nanotubes are disclosed by Kam et. al. (2004) Am. Chem. Soc, 126 (22):6850-6851, Liu et. al. (2009) Nano Lett, 9(3): 1007-1010, and Khodakovskaya et. al. (2009) ACS Nano, 3(10):3221-3227. Physical force agents can include heating, chilling, the application of positive pressure, or ultrasound treatment. Embodiments of the method can optionally include an incubation step, a neutralization step (e.g., to neutralize an acid, base, or oxidizing agent, or to inactivate an enzyme), a rinsing step, or combinations thereof. The methods of the invention can further include the application of other agents which will have enhanced effect due to the silencing of certain genes. For example, when a polynucleotide is designed to regulate genes that provide herbicide resistance, the subsequent application of the herbicide can have a dramatic effect on herbicide efficacy.

Agents for laboratory conditioning of a plant cell to permeation by polynucleotides include, e.g., application of a chemical agent, enzymatic treatment, heating or chilling, treatment with positive or negative pressure, or ultrasound treatment. Agents for conditioning plants in a field include chemical agents such as surfactants and salts.

In an aspect, a transformed or transfected cell is a plant cell. Recipient plant cell or explant targets for transformation include, but are not limited to, a seed cell, a fruit cell, a leaf cell, a cotyledon cell, a hypocotyl cell, a meristem cell, an embryo cell, an endosperm cell, a root cell, a shoot cell, a stem cell, a pod cell, a flower cell, an inflorescence cell, a stalk cell, a pedicel cell, a style cell, a stigma cell, a receptacle cell, a petal cell, a sepal cell, a pollen cell, an anther cell, a filament cell, an ovary cell, an ovule cell, a pericarp cell, a phloem cell, a bud cell, or a vascular tissue cell. In another aspect, this disclosure provides a plant chloroplast. In a further aspect, this disclosure provides an epidermal cell, a guard cell, a trichome cell, a root hair cell, a storage root cell, or a tuber cell. In another aspect, this disclosure provides a protoplast. In another aspect, this disclosure provides a plant callus cell. Any cell from which a fertile plant can be regenerated is contemplated as a useful recipient cell for practice of this disclosure. Callus can be initiated from various tissue sources, including, but not limited to, immature embryos or parts of embryos, seedling apical meristems, microspores, and the like. Those cells which are capable of proliferating as callus can serve as recipient cells for transformation. Practical transformation methods and materials for making transgenic plants of this disclosure (e.g., various media and recipient target cells, transformation of immature embryos, and subsequent regeneration of fertile transgenic plants) are disclosed, for example, in U.S. Pat. Nos. 6,194,636 and 6,232,526 and U.S. Patent Application Publication 2004/0216189, all of which are incorporated herein by reference. Transformed explants, cells or tissues can be subjected to additional culturing steps, such as callus induction, selection, regeneration, etc., as known in the art. Transformed cells, tissues or explants containing a recombinant DNA insertion can be grown, developed or regenerated into transgenic plants in culture, plugs or soil according to methods known in the art. In one aspect, this disclosure provides plant cells that are not reproductive material and do not mediate the natural reproduction of the plant. In another aspect, this disclosure also provides plant cells that are reproductive material and mediate the natural reproduction of the plant. In another aspect, this disclosure provides plant cells that cannot maintain themselves via photosynthesis. In another aspect, this disclosure provides somatic plant cells. Somatic cells, contrary to germline cells, do not mediate plant reproduction. In one aspect, this disclosure provides a non-reproductive plant cell.

### Nucleic Acids and Amino Acids

The use of the term “polynucleotide” or “nucleic acid molecule” is not intended to limit the present disclosure to polynucleotides comprising deoxyribonucleic acid (DNA). For example, ribonucleic acid (RNA) molecules are also envisioned. Those of ordinary skill in the art will recognize that polynucleotides and nucleic acid molecules can comprise deoxyribonucleotides, ribonucleotides, or combinations of ribonucleotides and deoxyribonucleotides. Such deoxyribonucleotides and ribonucleotides include both naturally occurring molecules and synthetic analogues. The polynucleotides of the present disclosure also encompass all forms of sequences including, but not limited to, single-stranded forms, double-stranded forms, hairpins, stem-and-loop structures, and the like. In an aspect, a nucleic acid molecule provided herein is a DNA molecule. In another aspect, a nucleic acid molecule provided herein is an RNA molecule. In an aspect, a nucleic acid molecule provided herein is single-stranded. In another aspect, a nucleic acid molecule provided herein is double-stranded.

As used herein, the term “recombinant” in reference to a nucleic acid (DNA or RNA) molecule, protein, construct, vector, etc., refers to a nucleic acid or amino acid molecule or sequence that is man-made and not normally found in nature, and/or is present in a context in which it is not normally found in nature, including a nucleic acid molecule (DNA or RNA) molecule, protein, construct, etc., comprising a combination of polynucleotide or protein sequences that would not naturally occur contiguously or in close proximity together without human intervention, and/or a polynucleotide molecule, protein, construct, etc., comprising at least two polynucleotide or protein sequences that are heterologous with respect to each other.

As used herein, the term “heterologous” refers to a nucleotide/polypeptide that originates from a foreign species, or, if from the same species, is substantially modified from its native form in composition and/or genomic locus by deliberate human intervention. A “heterologous” or a “recombinant” nucleotide sequence is a nucleotide sequence not naturally associated with a host cell into which it is introduced, including non-naturally occurring multiple copies of a naturally occurring nucleotide sequence.

In one aspect, methods and compositions provided herein comprise a vector. As used herein, the term “vector” refers to a DNA molecule used as a vehicle to carry exogenous genetic material into a cell.

In an aspect, one or more polynucleotide sequences from a vector are stably integrated into a genome of a plant. In an aspect, one or more polynucleotide sequences from a vector are stably integrated into a genome of a plant cell.

In an aspect, a first nucleic acid sequence and a second nucleic acid sequence are provided in a single vector. In another aspect, a first nucleic acid sequence is provided in a first vector, and a second nucleic acid sequence is provided in a second vector.

As used herein, the term “polypeptide” refers to a chain of at least two covalently linked amino acids. Polypeptides can be encoded by polynucleotides provided herein. An example of a polypeptide is a protein. Proteins provided herein can be encoded by nucleic acid molecules provided herein.

Nucleic acids can be isolated using techniques routine in the art. For example, nucleic acids can be isolated using any method including, without limitation, recombinant nucleic acid technology, and/or the polymerase chain reaction (PCR). General PCR techniques are described, for example in PCR Primer: A Laboratory Manual, Dieffenbach & Dveksler, Eds., Cold Spring Harbor Laboratory Press, 1995. Recombinant nucleic acid techniques include, for example, restriction enzyme digestion and ligation, which can be used to isolate a nucleic acid. Isolated nucleic acids also can be chemically synthesized, either as a single nucleic acid molecule or as a series of oligonucleotides. Polypeptides can be purified from natural sources (e.g., a biological sample) by known methods such as DEAE ion exchange, gel filtration, and hydroxyapatite chromatography. A polypeptide also can be purified, for example, by expressing a nucleic acid in an expression vector. In addition, a purified polypeptide can be obtained by chemical synthesis. The extent of purity of a polypeptide can be measured using any appropriate method, e.g., column chromatography, polyacrylamide gel electrophoresis, or HPLC analysis.

Without being limiting, nucleic acids can be detected using hybridization. Hybridization between nucleic acids is discussed in detail in Sambrook et. al. (1989, Molecular Cloning: A Laboratory Manual, 2nd Ed., Cold Spring Harbor Laboratory Press, Cold Spring Harbor, NY).

Polypeptides can be detected using antibodies. Techniques for detecting polypeptides using antibodies include enzyme linked immunosorbent assays (ELISAs), Western blots, immunoprecipitations and immunofluorescence. An antibody provided herein can be a polyclonal antibody or a monoclonal antibody. An antibody having specific binding affinity for a polypeptide provided herein can be generated using methods well known in the art. An antibody provided herein can be attached to a solid support such as a microtiter plate using methods known in the art.

The terms “percent identity” or “percent identical” as used herein in reference to two or more nucleotide or protein sequences is calculated by (i) comparing two optimally aligned sequences (nucleotide or protein) over a window of comparison, (ii) determining the number of positions at which the identical nucleic acid base (for nucleotide sequences) or amino acid residue (for proteins) occurs in both sequences to yield the number of matched positions, (iii) dividing the number of matched positions by the total number of positions in the window of comparison, and then (iv) multiplying this quotient by 100% to yield the percent identity. If the “percent identity” is being calculated in relation to a reference sequence without a particular comparison window being specified, then the percent identity is determined by dividing the number of matched positions over the region of alignment by the total length of the reference sequence. Accordingly, for purposes of the present application, when two sequences (query and subject) are optimally aligned (with allowance for gaps in their alignment), the “percent identity” for the query sequence is equal to the number of identical positions between the two sequences divided by the total number of positions in the query sequence over its length (or a comparison window), which is then multiplied by 100%. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity can be adjusted upwards to correct for the conservative nature of the substitution. Sequences that differ by such conservative substitutions are said to have “sequence similarity” or “similarity.”

The terms “percent sequence complementarity” or “percent complementarity” as used herein in reference to two nucleotide sequences is similar to the concept of percent identity but refers to the percentage of nucleotides of a query sequence that optimally base-pair or hybridize to nucleotides a subject sequence when the query and subject sequences are linearly arranged and optimally base paired without secondary folding structures, such as loops, stems or hairpins. Such a percent complementarity can be between two DNA strands, two RNA strands, or a DNA strand and a RNA strand. The “percent complementarity” can be calculated by (i) optimally base-pairing or hybridizing the two nucleotide sequences in a linear and fully extended arrangement (i.e., without folding or secondary structures) over a window of comparison, (ii) determining the number of positions that base-pair between the two sequences over the window of comparison to yield the number of complementary positions, (iii) dividing the number of complementary positions by the total number of positions in the window of comparison, and (iv) multiplying this quotient by 100% to yield the percent complementarity of the two sequences. Optimal base pairing of two sequences can be determined based on the known pairings of nucleotide bases, such as G-C, A-T, and A-U, through hydrogen binding. If the “percent complementarity” is being calculated in relation to a reference sequence without specifying a particular comparison window, then the percent identity is determined by dividing the number of complementary positions between the two linear sequences by the total length of the reference sequence. Thus, for purposes of the present application, when two sequences (query and subject) are optimally base-paired (with allowance for mismatches or non-base-paired nucleotides), the “percent complementarity” for the query sequence is equal to the number of base-paired positions between the two sequences divided by the total number of positions in the query sequence over its length, which is then multiplied by 100%.

For optimal alignment of sequences to calculate their percent identity, various pair-wise or multiple sequence alignment algorithms and programs are known in the art, such as ClustalW or Basic Local Alignment Search Tool (BLAST®), etc., that can be used to compare the sequence identity or similarity between two or more nucleotide or protein sequences. Although other alignment and comparison methods are known in the art, the alignment and percent identity between two sequences (including the percent identity ranges described above) can be as determined by the ClustalW algorithm, see, e.g., Chenna R. et. al., “Multiple sequence alignment with the Clustal series of programs,” Nucleic Acids Research 31: 3497-3500 (2003); Thompson J D et. al., “Clustal W: Improving the sensitivity of progressive multiple sequence alignment through sequence weighting, position-specific gap penalties and weight matrix choice,” Nucleic Acids Research 22: 4673-4680 (1994); Larkin M A et. al., “Clustal W and Clustal X version 2.0,” Bioinformatics 23: 2947-48 (2007); and Altschul, S. F., Gish, W., Miller, W., Myers, E. W. & Lipman, D. J. (1990) “Basic local alignment search tool.” J. Mol. Biol. 215:403-410 (1990), the entire contents and disclosures of which are incorporated herein by reference.

As used herein, a first nucleic acid molecule can “hybridize” a second nucleic acid molecule via non-covalent interactions (e.g., Watson-Crick base-pairing) in a sequence-specific, antiparallel manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymine (T), adenine (A) pairing with uracil (U), and guanine (G) pairing with cytosine (C). In addition, it is also known in the art that for hybridization between two RNA molecules (e.g., dsRNA), guanine base pairs with uracil. For example, G/U base-pairing is partially responsible for the degeneracy (i.e., redundancy) of the genetic code in the context of tRNA anti-codon base-pairing with codons in mRNA. In the context of this disclosure, a guanine of a protein-binding segment (dsRNA duplex) of a subject DNA-targeting RNA molecule is considered complementary to an uracil, and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementation, variables well known in the art. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or fewer nucleotides) the position of mismatches becomes important (see Sambrook et. al.). Typically, the length for a hybridizable nucleic acid is at least 10 nucleotides. Illustrative minimum lengths for a hybridizable nucleic acid are: at least 15 nucleotides; at least 18 nucleotides; at least 20 nucleotides; at least 22 nucleotides; at least 25 nucleotides; and at least 30 nucleotides). Furthermore, the skilled artisan will recognize that the temperature and wash solution salt concentration may be adjusted as necessary according to factors such as length of the region of complementation and the degree of complementation.

It is understood in the art that the sequence of polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined routinely using BLAST® programs (basic local alignment search tools) and PowerBLAST programs known in the art (see Altschul et. al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

### Regulatory Elements

Additional regulatory elements useful with this invention include, but are not limited to, introns, enhancers, termination sequences and/or 5′ and 3′ untranslated regions.

An intron useful with this invention can be an intron identified in and isolated from a plant and then inserted into an expression cassette to be used in transformation of a plant. As would be understood by those of skill in the art, introns can comprise the sequences required for self-excision and are incorporated into nucleic acid constructs/expression cassettes in frame. An intron can be used either as a spacer to separate multiple protein-coding sequences in one nucleic acid construct, or an intron can be used inside one protein-coding sequence to, for example, stabilize the mRNA. If they are used within a protein-coding sequence, they are inserted “in-frame” with the excision sites included. Introns may also be associated with promoters to improve or modify expression.

Non-limiting examples of introns useful with the present invention include introns from the ADHI gene (e.g., Adh1-S introns 1, 2 and 6), the ubiquitin gene (Ubi1), the RuBisCO small subunit (rbcS) gene, the RuBisCO large subunit (rbcL) gene, the actin gene (e.g., actin-1 intron), the pyruvate dehydrogenase kinase gene (pdk), the nitrate reductase gene (nr), the duplicated carbonic anhydrase gene 1 (Tdca1), the psbA gene, the atpA gene, or any combination thereof.

### Haploids/Haploid Induction Lines/Doubling Haploids.

In one aspect, methods and compositions are provided for transgenerational editing whereby the gene-editing components or CRISPR effector proteins under control of the selected constitutive promoters, as elsewhere described herein, are provided via haploid inducing lines, in order to simultaneously provide genome modification in haploid progeny, which can thereafter be subjected to chromosome doubling techniques. In this way, progeny plants which no longer contain the mutational elements after the desired edits have been made, that could further affect gene expression and/or create additional, unwanted mutations in the plant genome, can be eliminated in one step without having to resort to backcrossing schemes to select for that progeny which has lost the mutational elements but retain the desired edits incorporated in their genome.

Haploid induction can occur during self-pollination or intercrossing of two lines within the same species, or it can occur during wide crosses, where it can be viewed as a hybridization barrier, preventing the formation of interspecific hybrids. In maize, the most commonly employed method of inducing haploids is through the use of an intraspecific haploid inducer male line, which is primarily triggered by rearrangements of, mutations in, and/or recombinations, insertion, or deletions within a region of chromosome 1, specifically the MATRILINEAL (MATL) gene, also known as NOT LIKE DAD1 (NLD1) and PHOSPHOLIPASE A1 (PLA1) (with the notable exception of the ig type haploid induction, which is a result of a mutation in the INDETERMINATE GAMETOPHYTE1 gene on chromosome 3). In wheat, the most common method of inducting haploids is by wide cross to maize pollen—regardless of parent genotype or lineage, this works with almost any wheat crossed by almost any maize pollen.

HI maize lines contain a quantitative trait locus (“QTL”) on Chromosome 1 responsible for at least 66% of the variation in haploid induction. The QTL causes haploid induction at different rates when it is introgressed into various backgrounds. All maize haploid inducer lines used in the seed industry are derivatives of the founding HI line, known as Stock6, and all have the haploid inducer chromosome 1 QTL mutation.

As used herein, a “haploid” cell or nucleus comprises a single set of unpaired chromosomes (x). In contrast, a “diploid” cell or nucleus comprises two complete sets of chromosomes (2x) that are capable of homologous pairing. The haploid number of chromosomes can be represented by “n,” and the diploid number of chromosomes can be represented by “2n.” For example, in a diploid species such as corn, n=x=lO, and 2n=2x=20. A polyploid cell or nucleus comprises more than two complete sets of chromosomes. For example, some wheat lines are hexaploids, meaning they contain three sets of paired chromosomes (2n=6x=42). Both diploid and polyploid cells and nuclei can be reduced to haploid states.

As used herein, a “haploid plant” describes a sporophyte comprising a plurality of cells comprising a haploid nuclear genome. Occasionally, sectors of an otherwise haploid plant can spontaneously double to form diploid or polyploid sectors. The frequency of spontaneous chromosome doubling varies depending on the species. Rates of spontaneous chromosome doubling up to 70-90% in barley, up to 25-70% in wheat, up to 50-60% in rice, up to 50-90% in rye, and up to 20% in corn have been reported.

An haploid plant provided herein can be a maternal haploid plant, meaning it has lost its paternal nuclear genome while retaining its maternal nuclear genome. Alternatively, a haploid plant provided herein can be a paternal haploid plant, meaning it has lost its maternal nuclear genome while retaining its paternal nuclear genome. Typically, maternal mitochondria and plastid (e.g., chloroplast) genomes are retained in both maternal and paternal haploid plants.

Haploid plants provided herein can originate spontaneously, or they can be produced by using various haploid induction techniques. In one aspect, haploid plants provided herein are generated by pollinating a female plant with pollen from a “haploid induction” (HI) line of the same species. As used herein, a “haploid induction (HI) plant” is a plant capable of inducing haploidization in a progeny plant by eliminating one set of chromosomes. HI lines typically produce maternal haploids at low frequencies (<10%). As a non-limiting example, pollen from a plant of the haploid-inducing corn line Stock 6 can be used to generate maternal haploids in progeny plants via elimination of the Stock 6 chromosomes. As another non-limiting example, a corn plant harboring a mutation in the indeterminate gametophytel (igl) locus is capable of inducing paternal haploids upon fertilization via elimination of the maternal chromosomes; the maternal mitochondrial and plastid genomes are retained in igl-induced paternal haploids. As a further non-limiting example, pollen from a cotton plant harboring a mutation in the semigamy (se) locus is capable of producing either a paternal or a maternal haploid upon fertilization. However, haploid cotton plants are only generated when the maternal parent harbors the requisite se mutation. See, for example, Chaudhari. 1978. Bulletin of the Torrey Botanical Club. 105:98-103. In one aspect, this disclosure provides a HI plant. As another non-limiting example, it has been shown that manipulation of the centromere-specific histone CENH3 can induce the formation of haploids in Arabidopsis thaliana. See, for example, Ravi and Chan. 2010. Nature. 464:615-619. In one aspect, an HI line provided herein comprises a modified CENH3 protein. As another non-limiting example, it was also found that plants with loss of functional Msi2 protein due to a nucleotide polymorphism resulting in the introduction of a premature stop codon in the Msi2 protein, are able to induce haploid offspring after a cross to or with a wild type plant comprising a functional Msi2 protein (WO 2017058023 A1). In another aspect, an HI line provided herein comprises a modified Msi2 protein. As another non-limiting example, it was found that plants comprising modified CENPC protein comprising one or more active mutations which affect the functioning of CENPC protein yet allow plants expressing said modified CENPC protein to be viable, are able to induce haploid offspring after a cross to or with a wild type plant comprising an endogenous CENPC protein (WO 2017058022 A1). In another aspect, an HI line provided herein comprises a modified CENPC protein. As another non-limiting example, it was found that plants with a silenced patatin-like phospholipase 2A are able to induce haploid offspring (U.S. Pat. No. 9,677,082 B2). In another aspect, an HI line provided herein comprises a silenced patatin-like phospholipase 2A gene.

In one aspect, an HI plant provided herein is of a species selected from the group consisting of a corn plant, a rye plant, a wheat plant, a barley plant, a Tripsacum plant, a sorghum plant, a pearl millet plant, a soybean plant, an alfalfa plant, a sugarcane plant, a cotton plant, a canola plant, a potato plant, and a rice plant.

in another aspect, haploid induction can be achieved by pollinating a domesticated plant variety with pollen from a wild relative in an intragenic and/or interspecific cross. In yet another aspect, haploid induction is achieved by pollinating an egg cell of a first species from a first genus with pollen from a plant of a second species in a second genus in an “intergenic cross.” Such intragenic and intergenic crosses are often referred to as “wide crosses” or “wide hybridizations.” In one aspect, a wide cross provided herein results in the loss of the paternal nuclear genome. In another aspect, a wide cross provided herein results in the loss of the maternal nuclear genome. Those skilled in the art will recognize that in some instances hybrid progeny resulting from a wide cross must be backcrossed to the parent species comprising the desired nuclear genome in order to eliminate the nuclear genome of the second, undesired species. In one aspect, the first species in a wide cross is selected from the group consisting of wheat, rye, oat, barley, and Tripsacum, and the second species is corn. In another aspect, the first species in a wide cross is Tripsacum and the second species is corn. In another aspect, the first species in a wide cross is wheat, and the second species is corn. In another aspect, the first species in a wide cross is a wild species of barley and the second species is a domesticated species of barley. In another aspect, the first species in a wide cross is wheat, and the second species is selected from the group consisting of sorghum and pearl millet. In yet another aspect, the first species in a wide cross is a wild potato species (e.g., Solanum phreja), and the second species is a domesticated potato species. In another aspect, the first species in a wide cross is a species of the genus Orychophragmus and the second species is canola. In another aspect, the first species in a wide cross is Glycine tomentella and the second species is soybean. In another aspect, the first species in a wide cross is Oryza minuta and the second species is rice.

In one aspect, a haploid plant provided herein is produced by pollinating a plant using irradiated pollen. In another aspect, a haploid plant provided herein is produced in vitro. In another aspect, a maternal haploid plant provided herein is produced from the in vitro culturing of unpollinated female flower parts (e.g., ovules, placenta attached ovules, ovaries, whole flower buds). In yet another aspect, a paternal haploid plant provided herein is produced from the in vitro culturing of immature anthers.

In one aspect, in vitro embryo rescue is required to recover a haploid plant provided herein following a haploid induction event. In another aspect, a trait (e.g., color marker, such as an athocyanin marker like R1-nj, and/or an oil content marker, such as that described in PCT Application PCT/US2015/049344, titled Improved Methods of Plant Breeding Using High-Throughput Seed Sorting, filed Sep. 10, 2015 and corresponding U.S. patent application Ser. No. 14/206,238, the disclosure of each being incorporated by reference herein in their entirety, and/or a morphological marker capable of distinguishing haploid embryos from diploid embryos) is incorporated into a genome of a HI plant provided herein, a recipient plant provided herein, or both to facilitate the identification, differentiation and/or sorting of haploid embryos from diploid embryos. Haploid induction can be confirmed by the presence/absence of a phenotypic marker in the seed coat, aleurone, embryo, endosperm, or a combination thereof. As a non-limiting example, the corn R-nj color marker (R is a locus that conditions red and purple anthocyanin pigmentation), which colors the crown portion of the seed aleurone and the embryo red or purple, can be incorporated into a HI inducing corn line. When the HI line comprising R-nj is crossed as a male onto a colorless female line, haploid candidates can be selected by choosing seeds that have a R-nj pattern in the endosperm coupled with a colorless embryo. Haploid induction can also be confirmed by molecular markers that indicate a lack of heterogeneity. Such markers can be examined by techniques known in the art such as, without being limiting, sequence analysis (e.g., Sanger, 454, Illumina, Pac-Bio), PCR, Southern hybridization, fluorescence in situ hybridization (FISH), and ELISA.

Haploid plants often form aberrant floral structures and are unable to proceed through meiosis due to the absence of one set of homologous chromosomes. It is often desirable to convert a haploid plant to a diploid plant (a “doubled haploid”) in a process known as “haploid doubling” or “chromosome doubling.” Haploid doubling allows the generation of a plant that is homozygous at all loci in the nuclear genome in a single generation. In one aspect, a haploid plant provided herein is converted to a doubled haploid plant. In one aspect, a method of chromosome doubling provided herein comprises the use of a chromosome doubling agent selected from the group consisting of nitrous oxide (N20) gas, colchicine, oryzalin, amiprophosmethyl, trifluralin, caffeine, and pronamide. See for example, Doubled Haploid Production in Crop Plants: A Manual (Eds. M. Maluszynski, K. J. Kasha, B. P. Forster, and I. Szarejko (2003), Kluwer Academic Publishers); Prigge and Melchinger, 2012, Plant Cell Culture Protocols, 877: 161-172; and Kato and Geiger, 2002, Plant Breeding, 121: 370-377 (each of which are incorporated by reference herein in their entireties). In another aspect, a method of chromosome doubling provided herein comprises the use of colchicine. In yet another aspect, a method of chromosome doubling provided herein comprises the use of N20 gas. In still another aspect, a method of chromosome doubling provided herein comprises the use of colchicine or nitrous oxide gas. As used herein, when referring to chromosome count, “doubling” refers to increasing the chromosome number by a factor of two. For example, a haploid nuclear genome comprising 10 chromosomes is doubled to become a diploid nuclear genome comprising 20 chromosomes. As another example, a diploid nuclear genome comprising 20 chromosomes is doubled to become a tetraploid nuclear genome comprising 40 chromosomes. Confirmation of chromosome doubling can be carried out by FISH or other molecular biology techniques known in the art.

In one aspect, a haploid plant provided herein undergoes spontaneous chromosome doubling. Spontaneous chromosome doubling can produce diploid sectors that give rise to normal diploid floral structures. Such spontaneously doubled sectors are desirable because diploid floral structures resulting from spontaneous chromosome doubling produce normal eggs and pollen that can be self-pollinated or used to perform crosses with other plants.

### General

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

The following examples are included to demonstrate embodiments of the disclosure. It should be appreciated by those of skill in the art that many changes can be made in the specific embodiments which are disclosed and still obtain a like or similar result without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### EXAMPLES

### Example 1: Constructs for Constitutive Expression of Cas12a

This example describes the use of plant constitutive expression elements from the Zea mays Ubiquitin M1 gene, to drive the expression of Cas12a expression in order to generate diverse mutations at the R0 generation and in subsequent generations.

To test for the activity of the Cas12a nuclease, two independent corn target sites (TS) for LbCas12a gRNAs were selected. The two target sites are designated as TS9 (SEQ ID NO: 31) and TS10 (SEQ ID NO: 32). Two agrobacterium T-DNA constructs with gRNAs targeting each of these target sites were generated. pM102 had a functional cassette for the expression of Cas12a (also known as Cpf1) and a single gRNA targeting TS9. The Cas12a cassette comprised a Zea mays Ubiquitin 1 promoter, leader and intron sequence (Zm.UbgM1) (SEQ ID NO:1), operably linked 5′ to a plant codon optimized sequence for Lachnospiraceae bacterium Cas12a RNA-guided endonuclease (SEQ ID NO: 7). The protein sequence of LbCas12a is set forth as SEQ ID NO:8. The LbCas12a DNA sequence was flanked by DNA sequences encoding nuclear localization signal (NLS) sequences at the 5′ and 3′ ends (SEQ ID:9 and SEQ ID:10) and operably linked 5′ to a transcription terminator sequence from a rice Lipid transfer protein (LTP) gene (SEQ ID NO:11). The gRNA cassette comprising a synthetic Pol III promoter GSP2262 (SEQ ID NO: 12) operably linked to a transcribable sequence comprising, in order: a Cas12a-compatible Direct repeat (DR) sequence (SEQ ID NO: 30), spacer SP9 (SEQ ID NO: 34), DR and a poly (T)7 terminator.

pM704 had a functional cassette for the expression of Cas12 that was identical to the cassette in pM102 and two gRNA array expression cassettes that each comprised four gRNAs targeting TS10. gRNA cassette 1 comprised a synthetic Pol III promoter GSP2269 (SEQ ID NO: 35) operably linked to a transcribable sequence comprising, in order: a Cas12a-compatible Direct repeat (DR) sequence (SEQ ID NO: 30), spacer SP10 (SEQ ID NO: 35), a DR, SP11 (SEQ ID NO: 36), DR, SP12 (SEQ ID NO: 37), DR, SP13 (SEQ ID NO: 38), DR and a poly (T)₇ terminator. The transcribable portion of the transcript is a pre-crRNA precursor RNA that can be processed by Cas12a into four copies of mature SP10, SP11, SP12 and SP13 guide RNAs. gRNA cassette 2 comprised a synthetic Pol III promoter GSP2269 (SEQ ID NO: 35) operably linked to a transcribable sequence comprising, in order: a Cas12a-compatible Direct repeat (DR) sequence (SEQ ID NO: 30), spacer SP14 (SEQ ID NO: 39), a DR, SP15 (SEQ ID NO: 40), DR, SP16 (SEQ ID NO: 41), DR, SP17 (SEQ ID NO: 42), DR and a poly (T)₇ terminator. The transcribable portion of the transcript is a pre-crRNA precursor RNA that can be processed by Cas12a into four copies of mature SP14, SP15, SP16 and SP17 guide RNAs. Both the T-DNA vectors also comprised an expression cassette for the selectable marker CP4 conferring resistance to the herbicide glyphosate. The CP4 gene was expressed under the control of Oryza sativa Actin1 gene promoter.

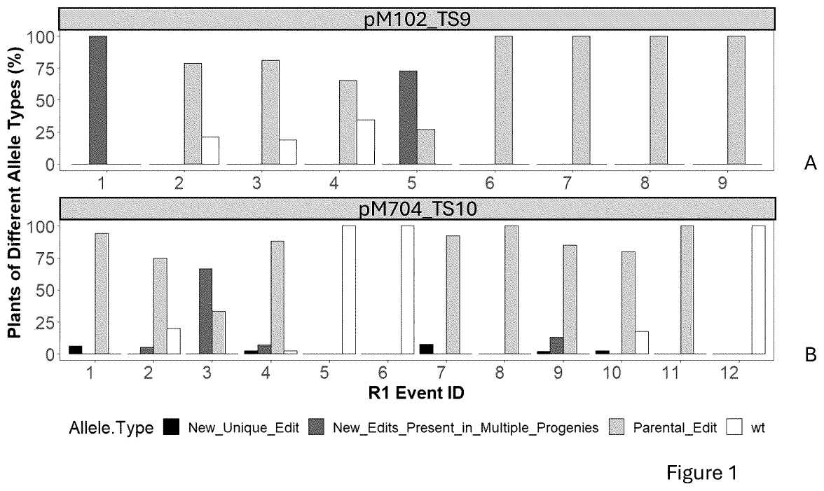

Corn 01DKD2 cultivar embryos were transformed with the vectors described above by Agrobacterium-mediated transformation and R0 plants were regenerated from the transformed corn cells. R0 plants expressing the two constructs were selfed, DNA was extracted from leaf samples from seven-day old R0 and R1 seedlings and the genomic target sites TS9 to TS10 were sequenced and analyzed for the presence of advanceable edits/indels. A plant is called “edited at an advanceable” level if at least ten percent of its sequence reads covering the target site carried insertions or deletions (InDels). As shown in FIG. 1, low post-R0 editing activities were observed in events generated with the two constructs as indicated by the number of new edits in R1 progenies that were not present in R0 parents. The new edits found in R1 progenies were furtherly grouped into 1) unique new edits, which were only present in a single R1 progeny; and 2) new but duplicated edits that were present in multiple R1 progenies, indicating they might be inherited from R0 parents. Using the new unique edits as the indicator, vertical transgenerational editing was detected in only one of the two target sites tested (pM704, TS10), and in only 6 out of 358 R1 plants. At the individual event level, new unique edits were present in 5 out of 12 events (pM704), accounting for 2-8% of the total plants per event. In contrast, none of events for the other target site (pM102, TS9), showed activities of vertical transgenerational editing.

### Example 2: Constructs for Constitutive Expression of Cas12a

This example describes the use of plant constitutive expression elements from the Zea mays Tubulin1 gene (Zm.Tubg1), Setaria italica Ubiquitin 1 gene (SETit.Ubq1), Saccharum officinarum Ubiquitin 4 gene (So.Ubq4), Oryza sativa Actin1 gene (OsAct) and Saccharum officinarum Ubiquitin 9 gene (So.Ubg9), to drive the expression of Cas12a expression so as to generate diverse mutations at the R0 generation and in subsequent generations.

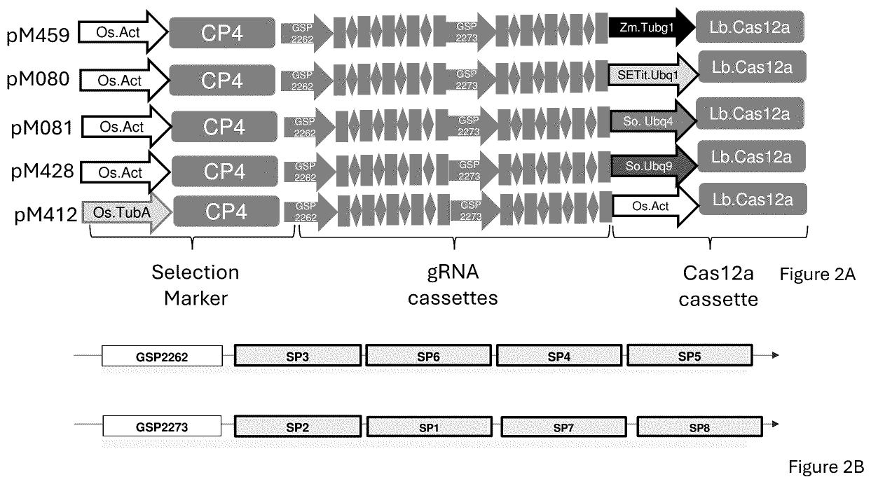

As shown in Table 1, and FIG. 2, five Agrobacterium T-DNA constructs were generated. Each construct comprised a LbCas12a nuclease cassette, two gRNA array cassettes, and a selectable marker cassette. The vectors are similar in design, except that the LbCas12a cassette is driven by various constitutive promoters.

TABLE 1
Cassettes designed for constitutive expression of Cas12a, in Corn.

| | | | Number of plants | | |
|---|---|---|---|---|---|
| | Promoter::LbCas12a::LTP | Promoter | sequenced (n) | | |
| Construct | term. | SEQ ID | R0 | R1 | R2 |
| pM459 | Zm.Tubg1::LbCas12a | 2 | 13 | 57 | 75 |
| pM080 | SETit.Ubq1::LbCas12a | 3 | 11 | 498 | 92 |
| pM081 | So.Ubq4::LbCas12a | 4 | 10 | 503 | 49 |
| pM428 | Os.Act::LbCas12a | 5 | 18 | 92 | 373 |
| pM142 | So.Ubq9::LbCas12a | 6 | 14 | 323 | NA |

To test for the activity of the Cas12a nuclease, eight independent corn target sites for LbCas112a gRNAs were selected. The gRNA spacers targeting these sequences have observed to have varying editing rates ranging from 35.9% to 100% as shown in Table 2.

TABLE 2
Cas12a targets sites, cognate gRNA spacers and editing activity.

| gRNA | Target | | Spacer | Observed |
|---|---|---|---|---|
| target site | SEQ | Spacer | SEQ | activity |
| name | ID NO | Name | ID NO | rate |
| TS1 | 14 | SP1 | 22 | 100% |
| TS2 | 15 | SP2 | 23 | 35.9% |
| TS3 | 16 | SP3 | 24 | 93% |
| TS4 | 17 | SP4 | 25 | 74.1% |
| TS5 | 18 | SP5 | 26 | 38.5% |
| TS6 | 19 | SP6 | 27 | 91.3% |
| TS7 | 20 | SP7 | 28 | NA |
| TS8 | 21 | SP8 | 29 | 40% |

Each vector described in Table 1 and FIG. 2A had a functional cassette for the expression of Cas12a (also known as Cpf1) comprising a promoter listed in Table 1, operably linked 5′ to a plant codon optimized sequence for Lachnospiraceae bacterium Cas12a RNA-guided endonuclease (SEQ ID NO: 7). The protein sequence of LbCas12a is set forth as SEQ ID NO:8. The LbCas12a DNA sequence was flanked by DNA sequences encoding nuclear localization signal (NLS) sequences at the 5′ and 3′ ends (SEQ ID:9 and SEQ ID:10) and operably linked 5′ to a transcription terminator sequence from a rice Lipid transfer protein (LTP) gene (SEQ ID NO:11).

Each construct comprised 2 gRNA array cassettes (see FIG. 2B). The first gRNA array expression cassette comprised a synthetic Pol III promoter GSP2262 (SEQ ID NO: 12) operably linked to a transcribable sequence comprising, in order: a Cas12a-compatible Direct repeat (DR) sequence (SEQ ID NO: 30), spacer SP3 (SEQ ID NO: 24), a DR, SP6 (SEQ ID NO:27), DR, SP4 (SEQ ID NO: 25), DR, SP5 (SEQ ID NO: 26), DR and a poly (T)₇ terminator. The transcribable portion of the transcript is a pre-crRNA precursor RNA that can be processed by Cas12a into four mature SP3, SP6, SP4 and SP5 guide RNAs. The second gRNA array expression cassette comprised a synthetic Pol III promoter GSP2273 (SEQ ID NO: 13) operably linked to a transcribable sequence comprising, in order: a Cas12a-compatible Direct repeat (DR) sequence (SEQ ID NO: 30), spacer SP2 (SEQ ID NO: 23), a DR, SP1 (SEQ ID NO: 22), DR, SP7 (SEQ ID NO: 28), DR, SP8 (SEQ ID NO: 29), DR and a poly (T)₇ terminator. The transcribable portion of the transcript is a pre-crRNA precursor RNA that can be processed by Cas12a into four copies of mature SP2, SP1, SP7 and SP8 guide RNAs. The T-DNA vector also comprised an expression cassette for the selectable marker CP4 conferring resistance to the herbicide glyphosate. In all but pM428 construct, the CP4 gene was expressed under the control of Oryza sativa Actin1 gene promoter. In pM428, the CP4 gene was expressed under the control of Oryza sativa TubulinA gene promoter

### Example 3: Protein Expression of LbCas12a in R0 Plants

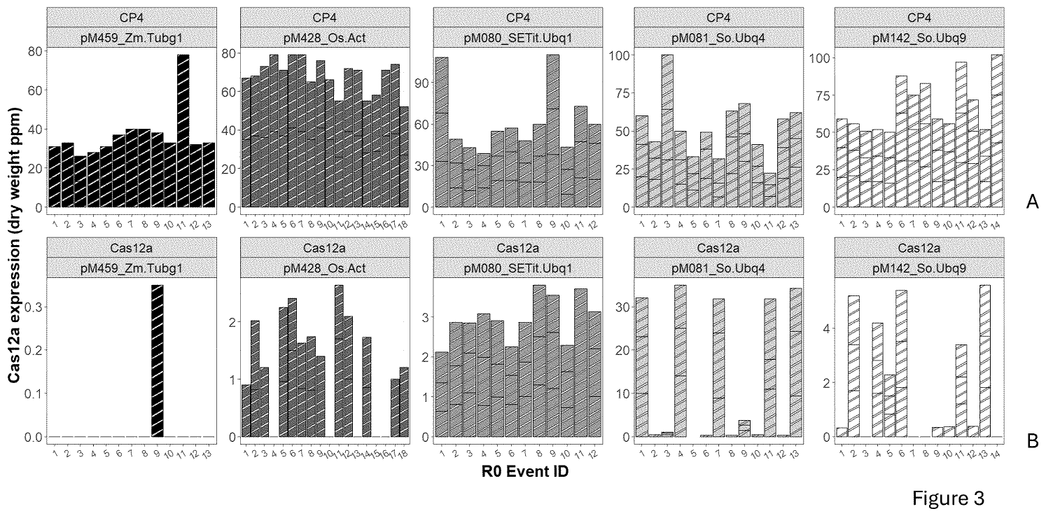

Corn 01DKD2 cultivar embryos were transformed with the vectors described above by Agrobacterium-mediated transformation and R0 plants were regenerated from the transformed corn cells. A Taqman® based assay was performed to identify the copy number of the Cas12a carrying construct. Plants with one or two copies of the Cas12a carrying construct were advanced. Total protein was extracted from 12 to 18 R0 plants and CP4 protein and LbCas12 protein expression levels were quantified using ELISA As shown in FIG. 3, while the expression of selection marker protein CP4 was stable across events carrying the different constructs and majority of the events fell within a similar range (largely 40-50 ppm), the expression of Cas12a driven by the five promoters varied significantly. So.Ubg4 was associated with the highest level of expression of up to 35 ppm, while Os.Act, SETit.Ubq1 and So.Ubg9 driven Cas12a showed moderate expression ranging from 0-5 ppm. The expression of Cas12a driven by the Zm.Tubg1 was lower than 0.5 ppm, which correlated with the low editing activities observed and described in Examples 4-5.

### Example 4: Vertical Transgenerational Editing in R0 and R1 Plants from Selfed Populations

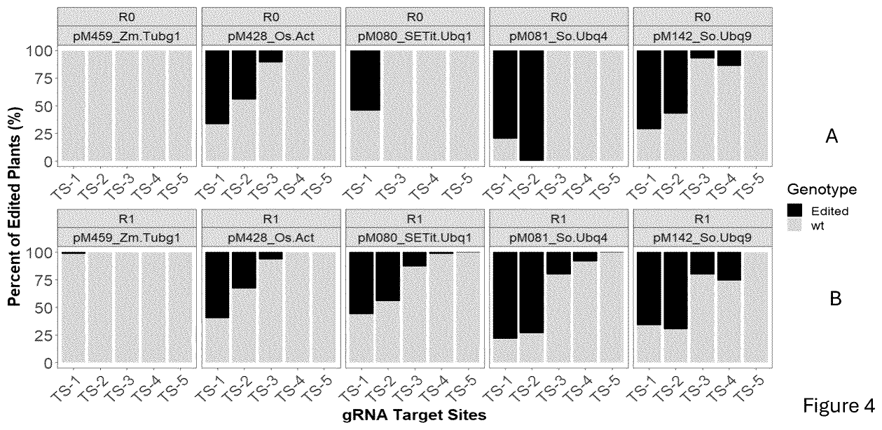

R0 plants expressing the five constructs described above were selfed for two generations to measure the transgenerational editing capability of the different Cas12a cassettes. Transgenerational editing ability of the CRISPR Cas12a system, is defined as the ability of the editing system to continually produce new edits after multiple generations of selfing (vertical) or outcrossing (horizontal). DNA was extracted from leaf samples from seven-day old R0 and R1 seedlings and the genomic target sites TS1 to TS5 were sequenced and analyzed for the presence of advanceable edits/indels. A plant is called “edited at an advanceable” level if at least ten percent of its sequence reads covering the target site carried insertions or deletions (InDels). As shown in FIG. 4, in the R0 and R1 generation, there was a clear distinction between the five constitutive promoters in terms of percent of edited plants across all the gRNA target sites. Up to 60-100% of edited plants were detected for the two high cutter gRNA (TS-1, TS-2) for Os.Act::LbCas12a, SETit.Ubq1, So.Ubg4; So.Ubg9 in R0 or R1 generation; while only 2% or less of the Zm. Tubg1 plants showed any edits. All promoters demonstrated editing capability, with SETit.Ubq1 and So.Ubg4 being the best performers showing editing activities across all the five gRNA target sites in R1 including a low efficiency gRNA for TS-5.

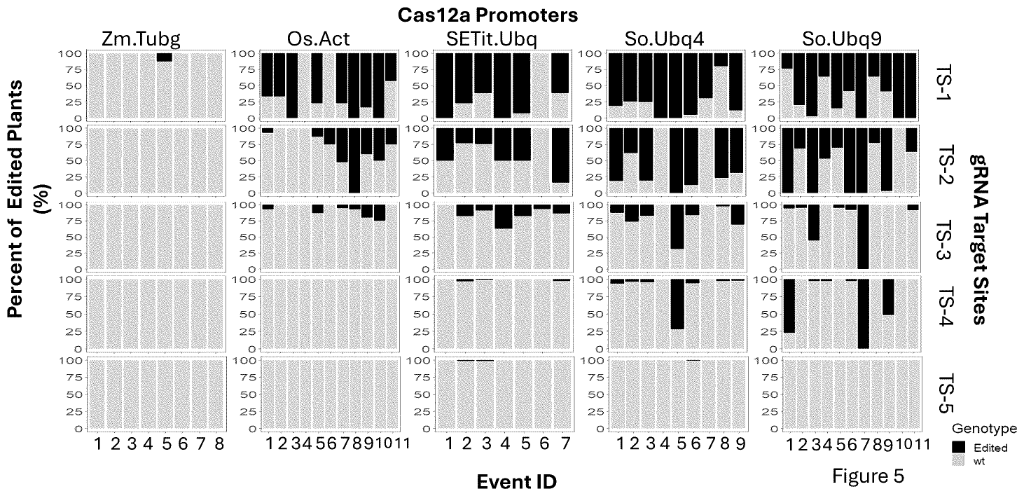

Between eight and eleven R1 events per construct were analyzed for editing efficiencies across five target sites (TS1 to TS5). The results are summarized in FIG. 5. This figure shows multiplexing editing capabilities of Cas12a at the five target sites within single events. Greater than 50% of the events from constructs pM428_Os.Act, pM080_SETit.Ubq1, pM081_So.Ubg4, pM142_So.Ubg9 showed edits in four to five target sites. pM459_Zm.Tubg1 was able to generate edits only in the high efficiency Target site TS1. However, the number of edited events were significantly fewer compared to the other promoter_Cas12a constructs. For example, 9 out of 11 events for pM428_Os.Act (82%); 6 out of 7 events for pM080_SETit.Ubq1 (86%); 9 out of 9 events for pM081_So.Ubg4 (100%), and 11 out of 11 events for pM142_So.Ubg9 (100%), showed edits at the TS1 site. In contrast, only 1 out of 8 events showed edits in the group of events with pM459_Zm.Tubg1 cassette. It was also observed that Cas12a driven by SoUbg4 and So.Ubg9 consistently showed a higher percent of edited plants across four of the five targets.

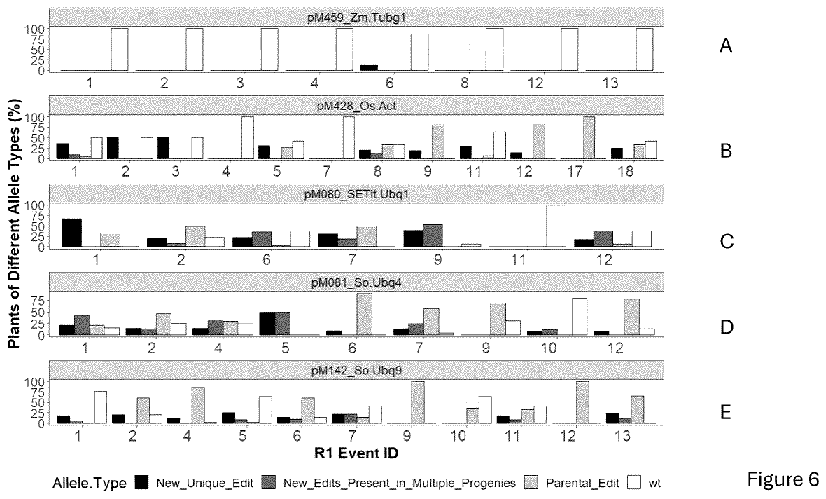

The next analysis was on the ability of the Cas12a expressed under the control of different promoters to drive the generation of new edits in post R0 generation. New edits are characterized as the presence of indels not observed in the parental R0 line. Edits in the TS1 target site from selfed R1 plants were analyzed and characterized for the presence of unique new edits. Eight to twelve individual R1 events were chosen for this analysis, 57 to 515 plants per construct were analyzed in total and the data are summarized in FIG. 6. Plants with new R1 edits were present in 70-90% of the events comprising cassettes with the 4 promoters: pM428_Os.Act, pM080_SETit.Ubq1, pM081_So.Ubg4, pM142_So.Ubg9, indicating these promoters were still driving high activity of Cas12a thereby enabling continued production of new edits. However only 1 out of 8 R1 events from the pM459_Zm.Tubg1 test group, was found to have unique new edits that were not observed in R0 generation.

For each R1 event, the proportion of plants with new edits in the R1 generation were also significantly higher for the four promoter constructs pM428_Os.Act, pM080_SETit.Ubq1, pM081_So.Ubg4 and pM142_So.Ubg9 than the proportion of plants with new edits in the R1 generation historically observed with Zm.Ubq1 as described in Example 1. Only 2 to 8% of plants had unique new edits in the R1 generation comprising the Zm.Ubq1::Cas12a cassette. In comparison, 14-50% of plants from pM428_Os.Act, 17-67% of plants from pM080_SETit.Ubq1, 8-50% of plants from pM081_So.Ubg4, and 11-25% of plants from pM142_So.Ubg9 comprised unique new edits.

Taken together the data suggests that significantly higher post R0 editing activity was observed for pM428_Os.Act, pM080_SETit.Ubq1, pM081_So.Ubg4, and pM142_So.Ubg9 promoters.

### Example 5: Transgenerational Editing in R0 and R1 Plants from Out-Crossed Population (Horizontal Transgenerational Editing)

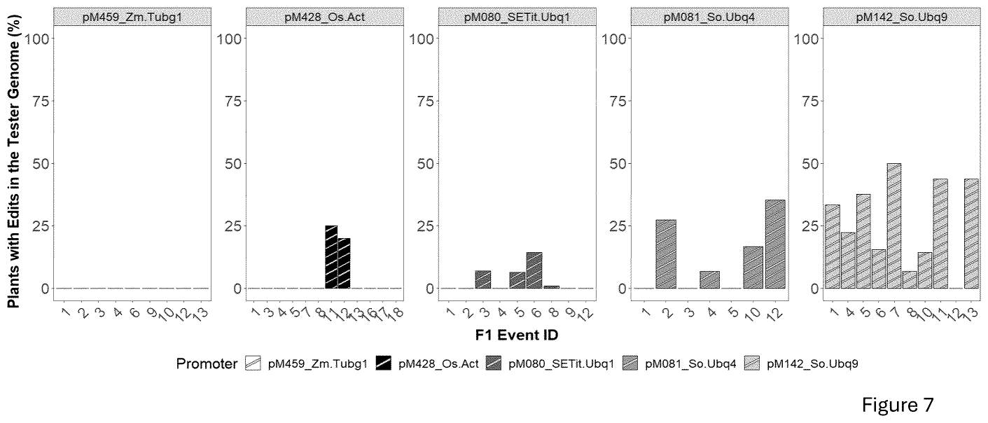

01DKD2 cultivar R0 plants expressing the pM428_Os.Act, pM080_SETit.Ubq1, pM081_So.Ubg4, pM142_So.Ubg9 and pM459_Zm.Tubg1 constructs described above were outcrossed to a tester line. Between seven to twelve unique events were chosen for each construct. DNA was extracted from seven-day old F1 seedlings and the TS1 genomic target was analyzed to measure the transgenerational editing capability of the Cas12a cassettes with different promoters in the F1 hybrids. TS3 was specifically chosen since it is the only target site with sequence diversity that would allow the sequencing analysis to distinguish between edits that are specific to either the genome of the original 01DKD2 transformation germplasm or the tester germplasm. Since the gRNA targeting TS3 was known to have moderate editing efficacy, for this analysis plants comprising edited sequence reads in at least one percent of the total edits were considered. As shown in FIG. 7, pM428_Os.Act, pM080_SETit.Ubq1, pM081_So.Ubg4, pM142_So.Ubg9 constructs demonstrated the ability to produce new edits in the tester genome through crossing, with the two stronger promoters pM081_So.Ubg4, pM142_So.Ubg9 showing much higher horizontal transgenerational editing rates than the other two moderate promoters pM428_Os.Act, pM080_SETit.Ubq1. Horizontal transgenerational editing within the tester genome was observed in 2 out of 12 events for pM428_Os.Act (17%); 4 out of 9 events for pM080_SETit.Ubq1 (44%); 4 out of 7 events for pM081_So.Ubg4 (57%), and 9 out of 10 events for pM142_So.Ubg9 (90%), respectively. No edits were observed in the tester genome in any of the F1 hybrids expressing the pM459_Zm.Tubg1 construct.

### Example 6. Constitutive Expression of Cas12a to Enable Editing from a Haploid Induction Line to a Target Genome

One cross editing (1XE) features a novel trait deployment system that combines the gene editing and doubled haploid technologies, to enable the efficient integration of edited traits into diverse elite germplasms through crossing. 1XE functions by crossing a transgenic ‘editing haploid inducer line’ (with Cas12a and gRNA) with an elite line resulting in transgene-free edited haploids. Colchicine can then be used to double the genome resulting in edited, transgene free diploids. Thus, through 1XE, it is possible to have a homozygous edit fixed in the elite genomic background within two to three generations instead of eight to nine generations via conventional crossing and editing. An “editing haploid inducer” line as described here contains any mutations/alleles necessary to cause haploid induction as well as the Cas12a cassette and gRNAs to make targeted edits. The successful deployment of 1XE for efficient crop trait improvement requires high transgenerational editing ability of the CRISPR system.

Homozygous R1 events comprising pM428_Os.Act, pM080_SETit.Ubq1, pM081_So.Ubg4 and pM459_Zm.Tubg1 constructs were crossed to a haploid inducer line to generate an “editing inducer” line. The editing inducer lines from each construct were subsequently crossed to two tester lines—93IDI3 or OH43 and the resulting haploid progeny were screened for the presence of edits and the type of edits within TS1, TS2, TS3, TS4, and TS5. The results are summarized in Table 3.

TABLE 3
Summary of edited haploids from 1XE testcross screens, pooled of
three rounds. Edit calls indicate unique edits. Plant IDs with
* indicate events comprising edits in two different loci/genes. .

| | | | Percent | | | |
|---|---|---|---|---|---|---|
| | | | of edited | | | |
| Plant_ID | Target | Edit_call | seq reads | Promoter | Event ID | Tester |
| _016 | TS-1 | S2d23 | 98.47 | pM080_SET.it.Ubq1 | S22899322 | 93IDI3 |
| _020 | TS-5 | S9d11 | 10.39 | | S22899366 | 93IDI3 |
| _067 | TS-1 | S15d17 | 99.59 | | S22899366 | 93IDI3 |
| _124 | TS-1 | S14d4 | 29.19 | | S22899322 | 93IDI3 |
| _151* | TS-1 | S17d3 | 25.26 | | S22899366 | OH43 |
| _151* | TS-2 | S5d7 | 13.08 | | S22899366 | OH43 |
| _046 | TS-1 | S13d23 | 99.52 | pM081_So.Ubq4 | S22899261 | 93IDI3 |
| _067* | TS-1 | S13d23 | 70.06 | | S22899261 | 93IDI3 |
| _067* | TS-1 | S18d5 | 10 | | S22899261 | 93IDI3 |
| _067* | TS-3 | S2d23 | 72.98 | | S22899261 | 93IDI3 |
| _104* | TS-1 | S9d18 | 99.95 | | S22899281 | 93IDI3 |
| _104* | TS-3 | S13d12 | 99.8 | | S22899281 | 93IDI3 |
| _122 | TS-1 | S15d10 | 99.66 | | S22899281 | 93IDI3 |
| _134a | TS-1 | S13d14 | 99.82 | | S22899281 | 93IDI3 |
| _134b | TS-1 | S2d23 | 99.95 | | S22899281 | 93IDI3 |
| _155 | TS-1 | S13d23 | 10.64 | | S22899281 | OH43 |
| _258 | TS-1 | S13d12 | 29.57 | | S22899261 | 93IDI3 |
| _001 | TS-4 | S8s1 | 10.97 | pM428_Os.Act | S22974048 | 93IDI3 |
| _042* | TS-1 | S22d23 | 52.1 | | S22974071 | 93IDI3 |
| _042* | TS-2 | S3d8 | 46.7 | | S22974071 | 93IDI3 |
| _059 | TS-1 | S14d11 | 27.76 | | S22974103 | 93IDI3 |

16 edited haploids with advanceable edit rates were recovered. Five edited haploids were recovered from editing inducer lines comprising the pM080_SETit.Ubq1 construct. Three edited haploids were recovered from editing inducer lines comprising the pM428_Os.Act construct. Eight edited haploids were recovered from editing inducer lines comprising the pM8 So.Ub4. In contrast, no edited haploid was recovered from editing inducer lines comprising the pM459 Zm.Tubg1 construct.

Four out of sixteen edited haploids harbored edits in two different loci/genes, demonstrating multiplex editing in haploids.

TABLE 4
Summary of editing characters of different promoters driving Cas12a expression.

| | | | | | | 1XE | 1XE |
|---|---|---|---|---|---|---|---|
| | | | | | | Testcross | Testcross |
| | R0 | R1 | R1 | R1 | Expression | Haploid | Diploid |
| | Edited | Edited | Edited | Edited | Profile, Cas12a | Editing | Editing |
| Promoter | Plants* | Plants* | Events* | Targets | protein levels | Rate (%)* | Rate (%)* |
| Zm.Tubg1 | 0/13 = 0% | 1/57 = 2% | 1/8 = 13% | 1/5 | Constitutive, <0.5 | 0/10 = 0% | 0/30 = 0% |
| | | | | | ppm | | |
| Os.Act | 12/18 = 67% | 55/92 = 60% | 9/11 = 82% | 3/5 | Constitutive, 0-3 | 2/110 = 1.8% | 51/59 = 86.4% |
| | | | | | ppm | | |
| SETit.Ubq1 | 6/11 = 55% | 281/498 = 56% | 6/7 = 86% | 5/5 | Constitutive, 0-4 | 4/122 = 3.3% | 40/62 = 64.5% |
| | | | | | ppm | | |
| So.Ubq4 | 8/10 = 80% | 394/503 = 78% | 9/9 = 100% | 5/5 | Constitutive, 0-35 | 7/143 = 4.9% | 84/106 = 79.2% |
| | | | | | ppm | | |
| So.Ubq9 | 10/14 = 71% | 214/323 = 66% | 11/11 = 100% | 4/5 | Constitutive, 0-5 | Not tested | Not tested |
| | | | | | ppm | | |

*indicates when summary was based on analysis of TS1 edit data.

Taken together, the data described in Examples 2 to 6 provide multiple layers of evidence demonstrating high transgenerational editing activities of Cas12a when expressed from the promoters SETit.Ubq1, So.Ubg4, Os.Act and So.Ubg9 with high editing rates across multiple events and multiple target sites. Additionally the SETit.Ubq1, So.Ubg4, Os.Act promoters were also successful in generating edited haploids when expressed from haploid inducer lines.

1. A method of editing a genome of a plant comprising:
(a) introducing into a plant cell:
(i) a first nucleic acid encoding a CRISPR effector protein operably linked to a heterologous first constitutive promoter a) which is selected from the group consisting of a plant constitutive promoter from the Zea mays Tubulin1 gene (Zm.Tubg1), a plant constitutive promoter from the Setaria italica Ubiquitin 1 gene (SETit.Ubq1), a plant constitutive promoter from the Saccharum officinarum Ubiquitin 4 gene (So.Ubg4), a plant constitutive promoter from the Oryza sativa Actin1 gene (Os.Act) and a plant constitutive promoter from the Saccharum officinarum Ubiquitin 9 gene (So.Ubg9); or b) comprises a nucleotide sequence that is at least 85% identical, at least 90% identical, at least 95% identical, at least 98% identical, at least 99% identical, or 100% identical to a nucleotide sequence selected from the group consisting of SEQ ID NOs:2-6 or a functional fragment thereof; and
(ii) a second nucleic acid encoding at least one guide nucleic acid operably linked to a heterologous second promoter, wherein at least one guide nucleic acid is capable of hybridizing to a target sequence within the plant genome; and
(b) regenerating at least one plant from the plant cell of step (a),
wherein the CRISPR effector protein and at least one guide nucleic acid form a ribonucleoprotein within at least one cell of the plant, and wherein the ribonucleoprotein generates at least one modification within the target sequence in at least one cell of the plant.

2. (canceled)

3. The method according to claim 1, wherein the CRISPR effector protein a) is selected from the group of Cas9, Cas12a, Cas12b and Cas X; or b) comprises an amino acid sequence having at least 90% sequence identity or at least 95% sequence identity or is identical to the amino acid sequence of SEQ ID NO: 8; or c) is encoded by a nucleotide sequence having at least 90% sequence identity or at least 95% sequence identity or is identical to the nucleotide sequence of SEQ ID NO: 7.

4-5. (canceled)

6. The method according to claim 1, wherein the plant cell is a corn plant cell.

7. The method according to claim 1, wherein said plant regenerated from the plant cell is a haploid inducer.

8. The method according to claim 1, further comprising a step of crossing the plant regenerated from the plant cell, to generate a progeny plant, wherein a least one further modification is generated within a target sequence in the genome of the progeny plant.

9. A method of editing a genome of a plant comprising:
(a) crossing a first plant with a second plant, wherein the first plant comprises a first nucleic acid encoding a CRISPR effector protein operably linked to a heterologous first constitutive promoter a) which is selected from the group consisting of a plant constitutive promoter from the Zea mays Tubulin1 gene (Zm.Tubg1), a plant constitutive promoter from the Setaria italica Ubiquitin 1 gene (SETit.Ubq1), a plant constitutive promoter from the Saccharum officinarum Ubiquitin 4 gene (So.Ubg4), a plant constitutive promoter from the Oryza sativa Actin1 gene (Os.Act) and a plant constitutive promoter from the Saccharum officinarum Ubiquitin 9 gene (So.Ubg9); or b) comprises a nucleotide sequence that is at least 85% identical, at least 90% identical, at least 95% identical, at least 98% identical, at least 99% identical or 100% identical to a nucleotide sequence selected from the group consisting of SEQ ID NOs:2-6 or a functional fragment thereof; and wherein the second plant comprises a second nucleic acid encoding at least one guide nucleic acid operably linked to a heterologous second promoter, wherein the at least one guide nucleic acid is capable of hybridizing to a target sequence within the genome; and
(b) obtaining at least one embryo from the crossing of step (a), wherein the CRISPR effector protein and at least one guide nucleic acid form a ribonucleoprotein within at least one cell of the embryo, and wherein the ribonucleoprotein generates at least one modification within the target sequence in at least one cell of the embryo.

10. (canceled)

11. The method according to claim 9, wherein the CRISPR effector protein al is selected from the group of Cas9, Cas12a, Cas12b and Cas X; b) comprises an amino acid sequence having at least 90% sequence identity or at least 95% sequence identity or is identical to the amino acid sequence of SEQ ID NO: 8; or c) is encoded by a nucleotide sequence having at least 90% sequence identity or at least 95% sequence identity or is identical to the nucleotide sequence of SEQ ID NO: 7.

12-13. (canceled)

14. The method according to claim 9, wherein the plant cell is a corn plant cell.

15. The method according to claim 9, wherein said first plant is a haploid inducer.

16. The method according to claim 15, wherein said embryo is haploid.

17. The method according to claim 16, further comprising treating said haploid embryo or a plant obtained from said haploid embryo with a chromosome doubling agent.

18. A method of generating two or more progeny plants with unique edits from a single plant cell, the method comprising:
(a) introducing into the plant cell:
(i) a first nucleic acid encoding a CRISPR effector protein operably linked to a heterologous first promoter a) which is selected from the group consisting of a plant constitutive promoter from the Zea mays Tubulin1 gene (Zm.Tubg1), a plant constitutive promoter from the Setaria italica Ubiquitin 1 gene (SETit.Ubq1), a plant constitutive promoter from the Saccharum officinarum Ubiquitin 4 gene (So.Ubg4), a plant constitutive promoter from the Oryza sativa Actin1 gene (Os.Act) and a plant constitutive promoter from the Saccharum officinarum Ubiquitin 9 gene (So.Ubg9); or b) comprises a nucleotide sequence that is at least 85% identical, at least 90% identical, at least 95% identical, at least 98% identical, at least 99% identical or 100% identical to a nucleotide sequence selected from the group consisting of SEQ ID NOs:2-6 or a functional fragment thereof; and
(ii) a second nucleic acid encoding at least one guide nucleic acid operably linked to a heterologous second promoter, wherein the at least one guide nucleic acid is capable of hybridizing to a target sequence within the genome; and
(b) regenerating a first plant from the plant cell of step (a), wherein the CRISPR effector protein and at least one guide nucleic acid form a ribonucleoprotein within at least one cell of the first plant, and wherein the ribonucleoprotein generates at least one double-stranded break within the target sequence in the at least cell;
(c) pollinating the first plant of step (b);
(d) germinating two or more seeds produced from step (c) to produced two or more progeny plants with unique edits.

19. (canceled)

20. The method according to claim 18, wherein al the CRISPR effector protein is selected from the group of Cas9, Cas12a, Cas12b and Cas X; b) the CRISPR effector protein comprises an amino acid sequence having at least 90% sequence identity or at least 95% sequence identity or is identical to the amino acid sequence of SEQ ID NO: 8; or c) is encoded by a nucleotide sequence having at least 90% sequence identity or at least 95% sequence identity or is identical to the nucleotide sequence of SEQ ID NO: 7.

21-22. (canceled)

23. The method according to claim 18, wherein the plant cell is a corn plant cell.

24. The method according to claim 18, wherein said first plant is a haploid inducer.

25. A recombinant DNA comprising nucleic acid encoding a CRISPR effector protein operably linked to a heterologous first promoter selected from the group consisting of a plant constitutive promoter from the Zea mays Tubulin1 gene (Zm.Tubg1), a plant constitutive promoter from the Setaria italica Ubiquitin 1 gene (SETit.Ubq1), and a plant constitutive promoter from the Saccharum officinarum Ubiquitin 9 gene (So.Ubg9) or wherein the heterologous first constitutive promoter comprises a nucleotide sequence that is at least 85% identical, at least 90% identical, at least 95% identical, at least 98% identical, at least 99% identical or 100% identical to a nucleotide sequence selected from the group consisting of SEQ ID NOs:2-3 and 6 or a functional fragment thereof.

26. (canceled)

27. The recombinant DNA according to claim 25, wherein the CRISPR effector protein is selected from the group of Cas9, Cas12a, Cas12b and Cas X.

28. The recombinant DNA according to claim 25, further comprising a second nucleic acid encoding at least one guide nucleic acid operably linked to a heterologous second promoter, wherein the at least one guide nucleic acid is capable of hybridizing to a target sequence within the genome.

29. A plant cell, plant, plant part or seed comprising a recombinant DNA according to claim 25.

30. A plant embryo obtained by a method according to claim 9.
